(19) United States
(12) Patent Application Publication
(10) Pub. No.: US 2007/0087346 A1
(43) Pub. Date: Apr. 19, 2007

(54) Orthogonal gene switches

(76) Inventors: Gennaro Ciliberto, Rome (IT); Raffaele De Francesco, Marino (IT); Fattori Daniela, Genzano (IT); Paolo Gallinari, Rome (IT); Olaf Daniel Kinzel, Rome (IT); Uwe Koch, Rome (IT); Ester Muraglia, Rome (IT); Carlo Toniatti, Rome (IT)

Correspondence Address: MERCK AND CO., INC, P O BOX 2000, RAHWAY, NJ, 07065-0907, US

(21) Appl. No.: 10/576,919

(22) PCT Filed: Oct. 15, 2004
(86) PCT No.: PCT/EP04/11683

§ 371 Date: Nov. 20, 2006

Related U.S. Application Data
(60) Provisional application No. 60/514,362, filed on Oct. 24, 2003.

(51) Int. Cl. C40B 30/06; C40B 40/08; C12Q 1/68; G01N 33/53; C07H 21/04; C12P 21/06; C07K 14/705

(52) U.S. Cl. 435/6; 435/69.1; 435/320.1; 435/455; 435/325; 435/7.1; 530/350; 536/23.5

(57) ABSTRACT

The present invention relates to an orthogonal gene switch for regulating the expression of a desired gene. The gene switch comprises a chimeric transcription factor that does not respond to endogenous ligands, and a ligand that is capable of activating the chimeric transcription factor but not endogenous transcription factors. The present invention also relates to the method of constructing the orthogonal gene switch.

### CROSS-REFERENCE TO RELATED APPLICATIONS

This application claims the benefits of U.S. Provisional Applications Ser. No. 60/514,362 filed Oct. 24, 2003.

### FIELD OF THE INVENTION

The present invention relates to novel gene switches that do not interfere with normal functions of endogenous nuclear receptors.

### BACKGROUND OF THE INVENTION

Nuclear hormone receptor superfamily is the largest known family of eukaryotic transcription regulators. The superfamily includes steroid hormones receptors, such as glucocorticoid receptors (GR), androgen receptors (AR), mineralocorticoid receptors (MR), progesterone receptors (PR), estrogen receptors (ER), and nonsteroid hormones receptors, such as thyroid hormone receptors (TR), vitamin D receptors (VDR), and retinoic acid receptors (RAR), as well as orphan receptors whose ligands have not been found. The hormones, via binding to the corresponding receptors, play important roles in the regulation of complex physiological events, including key steps in development, maintenance of homeostasis, cellular proliferation, differentiation, and death.

Nuclear hormone receptor action has been elucidated in considerable detail in vertebrate systems at both the cellular and molecular levels. In the absence of ligand, nuclear receptors for steroid hormones are bound with Hsp90, Hsp70 and p59 to form inactive complexes. The complexes reside in the cytoplasm, except for the estrogen receptor complexes, which are present in the nucleus. Upon binding the hormone, the receptors release the Hsp90, Hsp70 and p59 molecules, and translocate to the nucleus. Once inside the nucleus the receptors form homodimers and bind to the hormone response elements (HREs) at the regulatory regions of the target genes, resulting in the activation or repression of the target genes. In contrast, nuclear receptors for nonsteroid hormones, are bound to their response elements in the form of heterodimers free of hsp proteins even in the absence of the hormones. The nuclear receptors are activated by binding nonsteroid hormones.

While the hormones are structurally diverse compounds, their receptors are highly structurally related proteins. Nuclear hormone receptors are modular proteins organized into structurally and functionally defined domains, including amino-terminal region, DNA-binding domain (DBD), and ligand-binding domain (LBD). The ligand (hormone) binding domain, having a length of about 300 amino acids, is located in the carboxy-terminal half of the receptors. The ligand-binding domain appears to fold into a complex structure, creating a specific hydrophobic pocket that surrounds the ligand. The LBD also contains sequences responsible for receptor dimerization, hsp associations (for steroid hormone receptors), ligand-dependent transactivation function, silencing/repressor function (when LBD binds to antagonists) and nuclear translocation signal.

Although gene therapy shows great promise for treatment of a variety of pathologies, its practical application is still hampered by a number of hurdles, one of which is the control of trans-gene expression in patients through the administration of an exogenous agent. The control system of trans-gene expression is often called “gene switch”.

Ideally, the exogenous agent of a gene switch should affect exclusively the activity of the trans-gene and trans-gene expression should not be affected by endogenous agents.

Many systems commonly used in the regulation of eukaryotic gene expression include prokaryotic or other non-human components. These systems include tetracycline-dependent system derived from E. coli Tet-repressor (TetR), ecdysone (Ec)-dependent system derived from Drosophila Ec receptor, and rapamycine-dependent system. Because of their non-human components, these systems are likely to be immunogenic in humans or other immunocompetent hosts.

Nuclear hormone receptors and their ligands are suitable candidates for gene switches. The expression of nuclear hormone receptors from a species should not be immunogenic in the same species. For example, the expression of human estrogen receptor should not be immunogenic in a human host. The administration of exogenous ligands such as hormones, as well as the expression of exogenous nuclear hormone receptors, however, may interfere with the normal functions of endogenous nuclear hormone receptors. On the other hand, the exogenous nuclear hormone receptors may respond to endogenous hormones.

In some instances, the ligand-binding domain of the nuclear hormone receptors were modified to decrease the mutual interference. For example, a chimeric transcription factor was constructed by fusing a carboxy-terminal deletion mutant of the ligand-binding domain (LBD) of the human progesterone receptor to GALA DNA-binding domain and VP16 activation domain. While not responsive to progesterone, the chimeric transcription factor was activated by RU486, a synthetic progesterone antagonist (Wang Y. et al. 1994, PNAS 91:8180-8184). However, the system include exogenous ligands which interfere with the activities of the corresponding nuclear hormone receptors. Hence, there is a need to develop novel gene switches that neither affect the normal functions of endogenous nuclear hormone receptors, nor are affected by endogenous hormones.

The references cited throughout the present application are not admitted to be prior art to the claimed invention.

### SUMMARY OF THE INVENTION

The present invention provides a polypeptide that comprises,

SAGDMRAANL WPSPLMIKRS KKNSLALSLT ADQMVSALLD
AEPPILYSEY DPTRPFSEAS MMGLLTNLAX₁ RELVHMINWA
KRVPGFVDLT LHDQVHLLEC AWMEILMIGX₂ VWRSMEHPGK
LLX₃APNLLLD RNQGKCVEGX₄ VEX₅FDMX₆LAT SSRFRMMNLQ
GEEFVCLKSI ILLNSGVYTF LSSTLKSLEE KDHIHRVLDK
ITDTLIHLMA KAGLTLQQQH QRLAQLLLIL SHIRHMSNKX₇
MEX₈LYSMKCK NVVPLYDLLL EMLDAHRLHA PTSRGGASVE
ETDQSHLATA GSTSSHSLQK YYITGEAEGF PATV

in the peptide, X₁=D or A; X₂₋₆ are each independently G, A, C, V, L, L; M, F, Y, or W; X₇=G or R; and X₈=H or V.

According to an embodiment of the present invention, in the polypeptide, X₂=L, M, or V; X₃=F or W; X₄=M, G or A; X₅=I, M, V, or L; X₆=L.

According to a preferred embodiment of the present invention, in the polypeptide, X₁=D; X₂=L, M, or V; X₃=F or W; X₄=M, G or A; X₅=I, M, V, or L; X₆=L; X₇=G; and X₆=H.

According to an embodiment of the present invention, the polypeptide comprises the amino acid sequence of SEQ ID NO: 2.

According to a preferred embodiment of the present invention, in the polypeptide, X₁=D; X₂=L, M, or V; X₃=F or W; X₄=G or A; X₅=I, M, V, or L; X₆=L; X₇=G; and X₈=H.

According to a further preferred embodiment of the present invention, the polypeptide comprises an amino acid sequence selected from the group consisting of SEQ ID NO: 3-15.

According to a preferred embodiment of the present invention, in the polypeptide, X₁=A; X₂=L, M, or V; X₃=F or W; X₄=G or A; X₅=I, L, M, V, or L; X₆=L; X₇=G; and X₈=V.

According to a further preferred embodiment of the present invention, the polypeptide comprises an amino acid sequence selected from the group consisting of SEQ ID NO: 16-28.

According to an embodiment of the present invention, in the polypeptide, X₁=D; X₂=L, M, or V; X₃=F or W; X₄=G or A; X₅=I, M, V, or L; X₆=L; X₇=R; and X₈=H.

According to a further preferred embodiment of the present invention, the polypeptide comprises an amino acid sequence selected from the group consisting of SEQ ID NO: 29-41.

The present invention provides a polynucleotide encoding the polypeptide.

The present invention also provides a transcription factor that comprises a DNA-binding domain, a ligand-binding domain comprising the polypeptide, and a transcription regulatory domain.

According to an embodiment of the present invention, the ligand-binding domain of the transcription factor comprises an amino acid sequence selected from the group consisting of SEQ ID NO: 16-41.

According to an embodiment of the present invention, the DNA-binding domain of the transcription factor is GALA minimal DNA-binding domain.

According to a preferred embodiment of the present invention, the DNA-binding domain of the transcription factor is the DNA-binding domain of HNF-1.

According to an embodiment of the present invention, the transcription regulatory domain of the transcription factor is VP16 minimal activation domain.

According to an alternative embodiment of the present invention, the transcription regulatory domain of the transcription factor is a portion of the activation domain of human p65.

According to a preferred embodiment of the present invention, the transcription factor comprises SEQ ID NO: 43.

The present invention provides a polynucleotide encoding the transcription factor.

The present invention also provides a host cell transformed with a composition comprising the transcription factor.

The present invention also provides a compound that binds to and activates the transcription factor.

According to a preferred embodiment of the present invention, the compound is selected from the group consisting of CMP1 and CMP4-38.

The present invention also provides an orthogonal gene switch for regulating the expression of a desired gene. The gene switch comprises the transcription factor, and a vector comprising the desired gene, and a regulatory region that is fused to the desired gene. The transcription factor is capable of binding to the regulatory region.

According to an embodiment of the present invention, the gene switch further comprises a compound that binds to the ligand-binding domain and activates the transcription factor.

According to a preferred embodiment of the present invention, the ligand-binding domain of the gene switch comprises an amino acid sequence selected from the group consisting of SEQ ID NO: 16-28, and the compound is selected from the group consisting of CMP1, CMP4, CMP5, and CMP11-38.

According to a preferred embodiment of the present invention, the ligand-binding domain of the gene switch comprises an amino acid sequence selected from the group consisting of SEQ ID NO: 29-41, and the compound is selected from the group consisting of CMP6-10.

The present invention further provides a method of making an orthogonal gene switch. The method comprises selecting a ligand-binding domain (LBD) from a nuclear hormone receptor, selecting an inactive analogue of the hormone; constructing a library of transcription factors comprising veneered variants of the selected LBD, which are created by mutating amino acid residues that hinder the binding of the selected inactive analogue to various amino acid residues that might facilitate the binding; and screening the library with the selected inactive analogue to select the transcription factors that are activated by the inactive analogue.

According to an embodiment of the present invention, the method of making an orthogonal gene switch further comprises introducing mutations into the veneered LBDs of the selected transcription factors to reduce their affinity to the hormone and the ligand-independent activity of the transcription factors.

According to another embodiment of the present invention, the method further comprises making inactive analogues that are capable of activating the transcription factors carrying the mutations.

According to an embodiment of the present invention, the nuclear hormone receptor used in the method is selected from the group consisting of estrogen receptor (ER), androgen receptor (AR), glucocorticoid receptor (GR), mineralocorticoid receptor (MR), progesterone receptor (PR), vitamin D₃ receptor (VDR), thyroid hormone receptor (TR), and retinoic acid receptor (RAR).

According to a preferred embodiment of the present invention, the nuclear hormone receptor used in the method is human estrogen receptor α (hERα).

According to a further preferred embodiment of the present invention, the nuclear hormone receptor used in the method comprises SEQ ID NO: 2.

According to an embodiment of the present invention, the inactive analogue used in the method is an inactive analogue of a nuclear hormone receptor-specific agonist or antagonist.

According to an embodiment of the present invention, the inactive analogue used in the method is an inactive analogue of hERα-specific agonist or antagonist.

According to a preferred embodiment of the present invention, the inactive analogue used in the method is an inactive analogue of a human estrogen receptor β (hERβ)-specific agonist or antagonist According to a further preferred embodiment of the present invention, the inactive analogue is CMP1.

According to a preferred embodiment of the present invention, the library used in the library is a yeast one hybrid system.

According to a preferred embodiment of the present invention, the transcription factor used in the method further comprises a GAL4 minimal DNA-binding domain (DBD) and a VP16 minimal activation domain (AD).

According to a further preferred embodiment of the present invention, the library of transcription factors used in the method contains veneered LBDs with their amino acid residues 391, 404, 421, 424, and 428 independently selected from the group consisting of Gly, Ala, Cys, Val, Ile, Leu, Met, Phe, Tyr, and Trp.

Other features and advantages of the present invention are apparent from the additional descriptions provided herein including the different examples. The provided examples illustrate different components and methodology useful in practicing the present invention. The examples do not limit the claimed invention. Based on the present disclosure the skilled artisan can identify and employ other components and methodology useful for practicing the present invention.

### BRIEF DESCRIPTION OF THE DRAWINGS

FIGS. 1A and 1B. The structures and binding specificities of estradiol and representative active (CMP 2 and 3) and inactive analogies (CMP 1, 4-10). MG-LBD corresponds to the hERα-LBD with L(384)M and M(421)G.

FIG. 2A. Schematic representation of the GAL4 DBD/hERα LBD/VP16AD chimeric transcription factors. FIGS. 2B and 2C. Transcriptional activation in yeast transformants expressing chimeras based on wt hERα-LBD (squares) or hERα-L(384)M-LBD (circles) by estradiol and the hERβ-specific compound CMP2. Dose-response curves of β-Galactosidase activity in the presence of increasing concentrations of E₂ (FIG. 2B) or CMP2 (FIG. 2C) were performed. EC₅₀ values were determined as described in Material and Methods.

FIG. 3. The design of the hERα-LBD mutant library: molecular models of the three rotamers of CMP1 with solvent accessible surface in the crystal structure of the hERβ binding pocket. The five amino acid residues mutagenized in the library are highlighted.

FIG. 4. DNA sequence analysis of plasmids rescued from the genetically-selected variants. The number of independent clones in which the same mutation array was found is indicated. The consensus mutation of M421 into a smaller amino acid (G or A) is consistent with the R^{9a} benzyl substituent of CMP1 adopting rotamer conformation 1.

FIGS. 5A-5C. Ligand-dependent transcriptional activity of the M(421)G selected mutant in the lacZ reporter yeast strain Y187. Dose-response curves of β-Galactosidase activity in the presence of increasing concentrations of the two fluorenone compounds CMP4 (FIG. 5B), CMP5 (FIG. 5A), or 2 (FIG. 5C) were determined for the L(384)M, M(421)G selected mutant (squares) and for the L(384)M parental clone (circles). EC₅₀ values were calculated as described in Material and Methods.

FIGS. 6A and 6B. Mutagenesis of the ER amino acid residues making contacts with the D-ring of estradiol. The dose-response curves of β-Galactosidase activity in the presence of increasing concentrations of estradiol (FIG. 6A) or CMP4 (FIG. 6B) were determined for the triple mutated L(384)M, M(421)G, H(524)V chimera (filled triangles) and for the double mutated L(384)M, M(421)G selected chimera (filled squares). EC₅₀ values were calculated as described in Material and Methods.

FIGS. 7A and 7B. The D(351)A mutation reduce the ligand-independent activity of transcription factor carrying the hERα LBD L(384)M, M(421)G, H(524)V. FIG. 7A. In yeast. Dose-response of β-Galactosidase activity in the presence of increasing concentrations of CMP4. FIG. 7B. In HeLa cells. Co-transfections of the indicated amounts of GAL4 DBD/hERα-LBD/VP16AD and GAL4 DBD/hERα-D(351)A,L(384)M,M(421)G,H(524)V-LBD/VP16AD expression vector DNA with 1 μg of 5GAL4UAS-pSEAP reporter plasmid DNA were performed as described in Material and Methods. 24 hours post-transfection the outlined ligands were added at a concentration of 1 μM and after additional 24 hours the supernatants were collected for SEAP enzymatic assay.

FIGS. 8A-8C. Transcriptional induction of the M(421)G selected mutant containing the additional G(521)R mutation in response to antagonistic fluorenone compounds in yeast. Dose-response curves of β-Galactosidase activity in the presence of increasing concentrations of the two fluorenone compounds CMP6 (FIG. 8B) or CMP7 (FIG. 8C) in comparison with 4-OH Tam (FIG. 8A) were determined for the L(384)M, M(421)G, G(521)R mutant (filled squares), for the L(384)M, G(521)R parental clone (circles) and for wt alpha (grey triangles). EC₅₀ values were calculated as described in Material and Methods.

FIG. 9A. A representative dose-response curve chimeric transcription factor HEA-2 with CMP8 compound. FIG. 9B. The responses of chimeric transcription factor HEA-2 to various compounds.

### DETAILED DESCRIPTION OF THE INVENTION

As used herein, “nuclear hormone receptor superfamily” refers to the superfamily of nuclear hormone receptors, whose primary sequence suggests that they are related to each other. Representative examples include receptors for the estrogen, progesterone, glucocorticoid, mineralocorticoid, androgen, thyroid hormone, retinoic acid, retinoid X, Vitamin D, COUP-TF, ecdysone, Nurr-1, and orphan receptors.

The nuclear hormone receptors are composed of an activation domain, a DNA-binding domain and a ligand-binding domain. The DNA-binding domain recognizes and binds to specific regulatory DNA sequence elements and the ligand-binding domain binds the specific biological compound (ligand) to activate the receptor.

As used herein, “ligand” refers to any compound that activates or represses the receptor, by interaction with (binding) the ligand-binding domain of the receptor.

As used herein, “hormone” refers the natural ligand of the nuclear hormone receptor.

As used herein, “agonist” is a compound that is capable of interacting with the nuclear hormone receptor to promote a transcriptional response. For example, estrogen is an agonist for the estrogen receptor. Compounds that mimic estrogen would be defined as nuclear hormone receptor agonists.

As used herein, “antagonist” is a compound that is capable of interacting with a nuclear hormone receptor and of blocking the activity of a receptor agonist.

As used herein, “inactive analogues” refer to compounds that are structurally related to the ligands of a selected nuclear hormone receptor, but do not bind to the ligand-binding domain of the receptor. Inactive analogues are normally not found in animals or humans.

As used herein, “genetic material” refers to contiguous fragments of DNA or RNA. The genetic material which is introduced into targeted cells according to the methods described herein can be any DNA or RNA. For example, the nucleic acid can be: (1) normally found in the targeted cells, (2) normally found in targeted cells but not expressed at physiologically appropriate levels in targeted cells, (3) normally found in targeted cells but not expressed at optimal levels in certain pathological conditions, (4) novel fragments of genes normally expressed or not expressed in targeted cells, (5) synthetic modifications of genes expressed or not expressed within targeted cells, (6) any other DNA which may be modified for expression in targeted cells and (7) any combination of the above.

As used herein, “nucleic acid cassette” refers to the genetic material of interest which can express a protein, or a peptide, or RNA after it is incorporated transiently, permanently or episomally into a cell. The nucleic acid cassette is positionally and sequentially oriented in a vector with other necessary elements such that the nucleic acid in the cassette can be transcribed and, when necessary, translated in the cells.

As used herein, “veneered ligand-binding domain” or “mutant variant” refers to a ligand-binding domain with such an alternation or alternations of the primary sequence of the ligand-binding domain (LBD) of a receptor such that it differs from the wild type or naturally occurring sequence. The alteration can be point mutation, insertion, or deletion.

The terms “chimeric” and “chimera” refers to fusion proteins and transcription factors, activators and repressors of the invention, to denote composition of components of different origin, in particular of different parent proteins. This is irrespective of any inter-species chimericity, and indeed, in preferred embodiments a chimeric transcription factor of the invention is composed only of human protein components.

As used herein, “plasmid” refers to a construction comprised of extrachromosomal genetic material, usually of a circular duplex of DNA that can replicate independently of chromosomal DNA. Plasmids are used in gene transfer as vectors.

As used herein, “vector” refers to a construction comprised of genetic material designed to direct transformation of a targeted cell. A vector contains multiple genetic elements positionally and sequentially oriented with other necessary elements such that the nucleic acid in a nucleic acid cassette can be transcribed and when necessary translated in the transfected cells. In the present invention the preferred vector comprises the following elements linked sequentially at appropriate distance for allowing functional expression: a promoter; a 5′ mRNA leader sequence; an initiation site; a nucleic acid cassette containing the sequence to be expressed; a 3′ untranslated region; and a polyadenylation signal.

As used herein the term “expression vector” refers to a DNA plasmid that contains all of the information necessary to produce a recombinant protein in a heterologous cell.

As used herein, “transformed” refers to transient, stable or persistent changes in the characteristics (expressed phenotype) of a cell by the mechanism of gene transfer. Genetic material is introduced into a cell in a form where it expresses a specific gene product or alters the expression or effect of endogenous gene products. One skilled in the art readily recognizes that the nucleic acid cassette can be introduced into the cells by a variety of procedures, including transfection and transduction.

As used herein, “transfection” refers to the process of introducing a DNA expression vector into a cell. Various methods of transfection are possible including microinjection, CaPO₄ precipitation, liposome fusion (e.g. lipofection) or use of a gene gun.

As used herein, “transduction” refers to the process of introducing recombinant virus into a cell by infecting the cell with a virus particle.

As used herein, “transient” relates to the introduction of genetic material into a cell to express specific proteins, peptides, or RNA, etc. The introduced genetic material is not integrated into the host cell genome or replicated and is accordingly eliminated from the cell over a period of time.

As used herein, “stable” refers to the introduction of genetic material into the chromosome of the targeted cell where it integrates and becomes a permanent component of the genetic material in that cell. Gene expression after stable transduction can permanently alter the characteristics of the cell leading to stable transformation.

As used herein, “persistent” refers to the introduction of genes into the cell together with genetic elements that enable episomal (extrachromosomal) replication. This can lead to apparently stable transformation of the characteristics of the cell without the integration of the novel genetic material into the chromosome of the host cell.

As used herein, “transcriptional activity” is a relative measure of the degree of RNA polymerase activity at a particular promotor.

As used herein, the abbreviations of amino acid residues are shown as follows:

| | | |
|---|---|---|
| Alanine | Ala | A |
| Arginine | Arg | R |
| Asparagine | Asn | N |
| Aspartate | Asp | D |
| Cysteine | Cys | C |
| Histidine | His | H |
| Isoleucine | Ile | I |
| Glutamine | Gln | Q |
| Glutamate | Glu | E |
| Glycine | Gly | G |
| Leucine | Leu | L |
| Lysine | Lys | K |
| Methionine | Met | M |
| Phenylalanine | Phe | F |
| Proline | Pro | P |
| Serine | Ser | S |
| Threonine | Thr | T |
| Tryptophan | Trp | W |
| Tyrosine | Tyr | Y |
| Valine | Val | V |

### I. A Strategy to Construct an Orthogonal Gene Switch System

Nuclear hormone receptors, as well as their engineered derivatives, have been used as gene switches. The major problem for the gene switches is the mutual interference between the gene switches and the endogenous gene regulation system. On the one hand, the administered exogenous ligands can bind to the endogenous nuclear hormone receptors and activate or repress their activities. On the other hand, the expressed exogenous nuclear hormone receptor can bind endogenous hormone and respond to internal stimuli.

Hence, in order for a gene switch to be free of mutual interference, the gene switch should include a novel transcription factor and an exogenous ligand with the following characteristics. The transcription factor binds the exogenous ligand, but not any endogenous hormones, while the exogenous ligand does not bind to any endogenous nuclear hormone receptors. The binding of the exogenous ligand activates or represses the novel transcription factor. In other words, the exogenous ligand is the only agonist or antagonist of the novel transcription factor. Such a gene switch free of mutual interference is also known as orthogonal gene switch.

As used herein, “do not bind” or “incapable of binding” refers to no detectable binding, or an insignificant binding, i.e., having a binding affinity much lower than that of the natural ligand. The affinity can be determined with competitive binding experiments that measure the binding of a receptor with a single concentration of labeled ligand in the presence of various concentrations of unlabeled ligand. Typically, the concentration of unlabeled ligand varies over at least six orders of magnitude. Through competitive binding experiments, IC₅₀ can be determined. As used herein, “IC₅₀” refers to the concentration of the unlabeled ligand that is required for 50% inhibition of the association between receptor and the labeled ligand. IC₅₀ is an indicator of the ligand-receptor binding affinity. Low IC₅₀ represents high affinity, while high IC₅₀ represents low affinity.

According to an embodiment of the present application, an orthogonal gene switch is constructed by developing inactive analogues of a known nuclear hormone as the exogenous ligand, and veneering the ligand-binding domain of the nuclear hormone receptor for the orthogonal transcription factor. The inactive analogues are incapable of binding to the receptor. The veneered ligand-binding domain is capable of binding the inactive analogue, but not its naturally occurring counterpart.

According to a preferred embodiment of the present application, a nuclear hormone receptor is selected to provide its ligand-binding domain (LBD), which is veneered for the orthogonal transcription factor. The structures of the LBD and its ligands and their interactions with are preferrably well understood.

Based on the understanding of the structures and interactions, inactive analogues of the ligands are synthesized and selected. The inactive analogues are compounds that are structurally related to the ligands of a selected nuclear hormone receptor, but do not bind to the ligand-binding domain of the receptor. Inactive analogues are normally not found in animals or humans.

The ligand-binding domain (LBD) of the receptor is then veneered to bind the selected inactive analogue and be activated by the binding. The LBD is also veneered to diminish its capability of binding the nuclear hormone and the ligand-independent activity of the transcription factor carying it.

According to a preferred embodiment of the present invention, a library of transcription factors with mutant variants of the LBD is constructed. The mutant variants are made by mutating amino acid residues that might hinder the binding of the inactive analogue into various amino acid residues that might facilitate the binding. The library is then screened with the selected inactive analogue to select the transcription factors that can be activated by the inactive analogue. The selected transcription factors contain veneered LBDs that are capable of binding the inactive analogue. Further mutations are then introduced into the LBDs to reduce its binding of the hormone, and the ligand-independent transcription activity.

The veneered LBDs can then be used to construct appropriate transcription factors for the orthogonal gene switch system, while the selected inactive analogue can be used as exogenous ligand for the system. A new series of exogenous ligands for the system can also be obtained by developing inactive analogues that fit into the structure of the veneered LBD.

According to an embodiment of the present application, the ligand-binding domain (LBD) is from a nuclear receptor selected from the group consisting of estrogen receptor (ER), androgen receptor (AR), glucocorticoid receptor (GR), mineralocorticoid receptor (MR), progesterone receptor (PR), vitamin D₃ receptor (VDR), thyroid hormone receptor (TR), and retinoic acid receptor (RAR). The structure of the LBD and the interaction with its ligands is preferably well understood to provide guidance for veneering the LBD.

According to a preferred embodiment of the present application, the ligand-binding domain (LBD) is from human estrogen receptor α (hERα). The amino acid sequence of hERα LBD is provided by SEQ ID NO: 1:

SAGDMRAANL WPSPLMIKRS KKNSLALSLT ADQMVSALLD
AEPPILYSEY DPTRPFSEAS MMGLLTNLAD RELVHMINWA
KRVPGFVDLT LHDQVHLLEC AWLEILMIGL VWRSMEHPGK
LLFAPNLLLD RNQGKCVEGM VEIFDMLLAT SSRFRMMNLQ
GEEFVCLKSI ILLNSGVYTF LSSTLKSLEE KDHIHRVLDK
ITDTLIHLMA KAGLTLQQQH QRLAQLLLIL SHIRHMSNKG
MEHLYSMKCK NVVPLYDLLL EMLDAHRLHA PTSRGGASVE
ETDQSHLATA GSTSSHSLQK YYITGEAEGF PATV

The ligand-binding domain (LBD) is located in the carboxy-terminal half of human estrogen receptor α(hERα), from amino acid residue 282 to amino acid residue 595 of the receptor. As used herein, amino acid residues or point mutations of hERα LBD are numbered according to their corresponding positions in the full-length hERα.

According to a further preferred embodiment of the present application, the hERα LBD carries a Leu(384)Met mutation. The L384M mutation is both necessary and sufficient to make hERα LBD have the binding specificities of hERβ LBD. The amino acid sequence of hERα LBD carrying the L384M mutation is provided by SEQ ID NO: 2:

SAGDMRAANL WPSPLMIKRS KKNSLALSLT ADQMVSALLD
AEPPILYSEY DPTRPFSEAS MMGLLTNLAD RELVHMINWA
KRVPGFVDLT LHDQVHLLEC AWMEILMIGL VWRSMEHPGK
LLFAPNLLLD RNQGKCVEGM VEIFDMLLAT SSRFRMMNLQ
GEEFVCLKSI ILLNSGVYTF LSSTLKSLEE KDHIHRVLDK
ITDTLIHLMA KAGLTLQQQH QRLAQLLLIL SHIRHMSNKG
MEHLYSMKCK NVVPLYDLLL EMLDAHRLHA PTSRGGASVE
ETDQSHLATA GSTSSHSLQK YYITGEAEGF PATV

### II. The Structures of the Exogenous Ligands and the Methods of Synthesizing Them

As discussed above, after a nuclear hormone receptor is selected to provide its ligand-binding domain (LBD), inactive analogues of the nuclear hormone are synthesized and selected to direct the veneering of the LBD for the othogonal gene switch system. New inactive analogues can also be synthesized to fit the structure of the veneered LBD. According to a preferred embodiment of the present application, an estrogen receptor is selected to provide its LBD, and the inactive analogues of estrogen are synthesized and selected.

According to a preferred embodiment of the present application, the inactive analogues are the compounds described by the following chemical formulae: 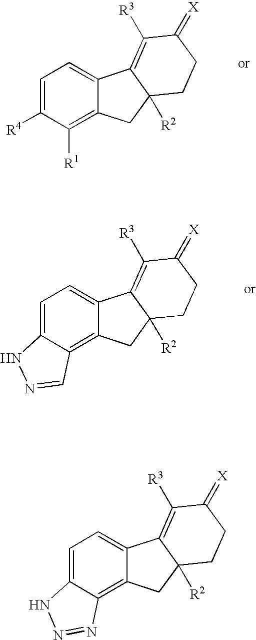
wherein X is selected from the group consisting of: O, N—OR^{a}, N—NR^{a}R^{b} and C₁₋₆ alkylidene, wherein said alkylidene group is unsubstituted or substituted with a group selected from hydroxy, amino, O(C₁₋₄alkyl), NH(C₁₋₄alkyl), or N(C₁₋₄alkyl)₂;
R¹ is selected from the group consisting of hydrogen, OR^{b}, NR^{b}R^{c}, fluoro, chloro, bromo, iodo, cyano, and nitro;
R² is selected from the group consisting of C₁₋₁₀alkyl, C₂₋₁₀alkenyl, C₂₋₁₀alkynyl, cycloalkylalkyl, arylalkyl and heteroarylalkyl, wherein said alkyl, alkenyl, alkynyl, cycloalkylalkyl, arylalkyl and heteroarylalkyl groups can be optionally substituted with a group selected from OR^{b}, SR^{b}, C(═O)R^{b}, 1-5 fluoro, chloro, iodo, cyano;
R³ is selected from the group consisting of hydrogen, chloro, bromo, iodo, cyano, NR^{a}R^{c}, OR^{a}, C(═O)R^{a}, CO₂R^{c}, CONR^{a}R^{c}, C₁₋₁₀alkyl, C₂₋₁₀alkenyl, C₂₋₁₀alkynyl, C₃₋₇cycloalkyl, cycloalkylalkyl, aryl, heteroaryl, arylalkyl, and heteroarylalkyl, wherein said alkyl, alkenyl, alkynyl, cycloalkyl, aryl and heteroaryl groups are either unsubstituted or independently substituted with 1, 2 or 3 groups selected from fluoro, chloro, bromo, iodo, cyano, OR^{a}, NR^{a}R^{c}, O(C═O)R^{a}, O(C═O)NR^{a}R^{c}, NR^{a}(C═O)R^{c}, NR^{a}(C═O)OR^{c}, C(═O)R^{a}, CO₂R^{a}, CONR^{a}R^{c}, CSNR^{a}R^{c}, SR^{a}, S(O)R^{a}, SO₂R^{a}, SO₂NR^{a}R^{c}, YR^{d}, and ZYR^{d};
R⁴ is selected from the group consisting of OR^{b}, OR^{a}, O(C═O)R^{c}, O(C═O)OR^{c}, and NH(C═O)R^{c};
R^{a} is selected from the group consisting of hydrogen, C₁₋₆alkyl, and phenyl, wherein said alkyl group can be optionally substituted with a group selected from hydroxy, amino, O(C₁₋₄alkyl), NH(C₁₋₄alkyl), N(C₁₋₄alkyl)₂, phenyl, or 1-5 fluoro;
R^{b} is selected from the group consisting of hydrogen, C₁₋₄alkyl, and phenyl;
R^{c} is selected from the group consisting of hydrogen and C₁₋₄alkyl;
   or R^{a} and R^{c}, whether or not on the same atom, can be taken together with any attached and intervening atoms to form a 4-6 membered ring;
R^{d} is selected from the group consisting of NR^{b}R^{c}, OR^{a}, CO₂R^{a}, O(C═O)R^{a}, NR^{c}(C═O)R^{b}, CONR^{a}R^{c}, SO₂NR^{a}R^{c}, and a 4-7 membered N-heterocycloalkyl ring that can be optionally interrupted by O, S, NR^{c}, or C═O;
Y is selected from the group consisting of CR^{b}R^{c}, C₂₋₆ alkylene and C₂₋₆ alkenylene, wherein said alkylene and alkenylene linkers can be optionally interrupted by O, S, or NR^{c};
Z is selected from the group consisting of O, S, NR^{c}, C═O, O(C═O), (C═O)O, NR^{c}(C═O) or (C═O)NR^{c}.

In the compounds of the present invention, X is preferably selected from the group consisting of O and N—OR^{a}. More preferably, X is selected from the group consisting of O, N—OH and N—OCH₃.

In the compounds of the present invention, R¹ is preferably selected from the group consisting of hydrogen, NR^{b}R^{c}, fluoro, chloro, bromo, nitro and C₁₋₄alkyl.

In the compounds of the present invention, R² is preferably selected from the group consisting of C₁₋₁₀alkyl, C₂₋₁₀alkenyl, C₃₋₆cycloalkyl, cycloalkylalkyl, arylalkyl and heteroarylalkyl, wherein said alkyl, alkenyl, cycloalkyl and cycloalkylalkyl, arylalkyl and heteroarylalkyl, groups can be optionally substituted with a group selected from OR^{b}, SR^{b}, C(═O)R^{b}, or 1-5 fluoro, chloro, iodo, cyano.

In the compounds of the present invention, R² is more preferably selected from the group consisting of arylalkyl and heteroarylalkyl, wherein said arylalkyl and heteroarylalkyl, groups can be optionally substituted with a group selected from OR^{b}, SR^{b}, C(═O)R^{b}, or 1-5 fluoro, chloro, iodo, cyano.

In the compounds of the present invention, R³ is preferably selected from the group consisting of hydrogen, chloro, bromo, iodo, cyano, OR^{a}, C(═O)R^{a}, C₁₋₁₀alkyl, C₂₋₁₀alkenyl, cycloalkylalkyl, aryl, heteroaryl, arylalkyl, and heteroarylalkyl, wherein said alkyl, alkenyl, aryl and heteroaryl groups are either unsubstituted or independently substituted with 1, 2 or 3 groups selected from fluoro, chloro, bromo, iodo, cyano, OR^{a}, NR^{a}R^{c}, O(C—O)NR^{a}R^{c}, C(═O)R^{a}, CO₂R^{c}, CONR^{a}R^{c}, CSNR^{a}R^{c}, SR^{a}, YR^{d}, and ZYR^{d}.

In the compounds of the present invention, R³ is more preferably selected from the group consisting of hydrogen, chloro, bromo, iodo, cyano, C₁₋₁₀alkyl, aryl, heteroaryl, wherein said alkyl, aryl and heteroaryl groups are either unsubstituted or independently substituted with 1, 2 or 3 groups selected from fluoro, chloro, bromo, cyano, NR^{a}C(═O)R^{c}, NR^{a}R^{c}, O(C═O)NR^{a}R^{c}, C(═O)R^{a}, CO₂R^{c}, CONR^{a}R^{c}, SR^{a}, YR^{d}, and ZYR^{d}.

In the compounds of the present invention, R⁴ is preferably selected from the group consisting of hydrogen, O(C═O)R^{c}, OR^{a} and O(C═O)OR^{c}.

In the compounds of the present invention, R⁴ is more preferably OH.

The compounds of the present invention can have chiral centers and occur as racemates, racemic mixtures, diastereomeric mixtures, and as individual diastereomers, or enantiomers with all isomeric forms being included in the present invention. Therefore, where a compound is chiral, the separate enantiomers, substantially free of the other, are included within the scope of the invention; further included are all mixtures of the two enantiomers.

The term “alkyl” shall mean a substituting univalent group derived by conceptual removal of one hydrogen atom from a straight or branched-chain acyclic saturated hydrocarbon (i.e., —CH₃, —CH₂CH₃, —CH₂CH₂CH₃, —CH(CH₃)₂, —CH₂CH₂CH₂CH₃, —CH₂CH(CH₃)₂, —C(CH₃)₃, etc.).

The term “alkenyl” shall mean a substituting univalent group derived by conceptual removal of one hydrogen atom from a straight or branched-chain acyclic unsaturated hydrocarbon containing at least one double bond (i.e., —CH═CH₂, —CH₂CH═CH₂, —CH═CHCH₃, —CH₂CH═C(CH₃)₂, etc.).

The term “alkynyl” shall mean a substituting univalent group derived by conceptual removal of one hydrogen atom from a straight or branched-chain acyclic unsaturated hydrocarbon containing at least one triple bond (i.e., —C≡CH, —CH₂C≡H, —C≡CCH₃, —CH₂C≡CCH₂(CH₃)₂, etc.).

The term “alkylene” shall mean a substituting bivalent group derived from a straight or branched-chain acyclic saturated hydrocarbon by conceptual removal of two hydrogen atoms from different carbon atoms (i.e., —CCH2CH2-, —CH2CH2CH2CH2-, —CH₂C(CH₃)₂CH₂—, etc.).

The term “alkylidene” shall mean a substituting bivalent group derived from a straight or branched-chain acyclic saturated hydrocarbon by conceptual removal of two hydrogen atoms from the same carbon atom (i.e., ═C₂, ═CHCH₃, ═C(CH₃)₂, etc.).

The term “alkenylene” shall mean a substituting bivalent group derived from a straight or branched-chain acyclic unsaturated hydrocarbon by conceptual removal of two hydrogen atoms from different carbon atoms (i.e., —CH═CH—, —CH₂CH═CH—, CH₂CH═CHCH₂—, —C(CH₃)═C(CH₃)—, etc.).

The term “cycloalkyl” shall mean a substituting univalent group derived by conceptual removal of one hydrogen atom from a saturated monocyclic hydrocarbon (i.e., cyclopropyl, cyclobutyl, cyclopentyl, cyclohexyl, or cycloheptyl).

The term “cycloalkenyl” shall mean a substituting univalent group derived by conceptual removal of one hydrogen atom from an unsaturated monocyclic hydrocarbon containing a double bond (i.e., cyclopentenyl or cyclohexenyl).

The term “heterocycloalkyl” shall mean a substituting univalent group derived by conceptual removal of one hydrogen atom from a heterocycloalkane wherein said heterocycloalkane is derived from the corresponding saturated monocyclic hydrocarbon by replacing one or two carbon atoms with atoms selected from N, O or S. Examples of heterocycloalkyl groups include, but are not limited to, oxiranyl, azetidinyl, pyrrolidinyl, piperidinyl, piperazinyl, and morpholinyl.

The term “aryl” as used herein refers to a substituting univalent group derived by conceptual removal of one hydrogen atom from a monocyclic or bicyclic aromatic hydrocarbon. Examples of aryl groups are phenyl, indenyl, and naphthyl.

The term “heteroaryl” as used herein refers to a substituting univalent group derived by the conceptual removal of one hydrogen atom from a monocyclic or bicyclic aromatic ring system containing 1, 2, 3, or 4 heteroatoms selected from N, O, or S. Examples of heteroaryl groups include, but are not limited to, pyrrolyl, furyl, thienyl, imidazolyl, pyrazolyl, oxazolyl, isoxazolyl, thiazolyl, pyridyl, pyrimidinyl, pyrazinyl, benzimidazolyl, indolyl, and purinyl.

The term “cycloheteroalkyl,” as used herein, shall mean a 3- to 8-membered fully saturated heterocyclic ring containing one or two heteroatoms chosen from N, O or S. Examples of cycloheteroalkyl groups include, but are not limited to piperidinyl, pyrrolidinyl, azetidinyl, morpholinyl, piperazinyl.

The term “alkoxy,” as used herein, refers to straight or branched chain alkoxides of the number of carbon atoms specified (e.g., C₁₋₅ alkoxy), or any number within this range (i.e., methoxy, ethoxy, etc.).

Whenever the term “alkyl” or “aryl” or either of their prefix roots appear in a name of a substituent (e.g., aryl C₁₋₈ alkyl) it shall be interpreted as including those limitations given above for “alkyl” and “aryl.” Designated numbers of carbon atoms (e.g., C₁₋₁₀) shall refer independently to the number of carbon atoms in an alkyl or cyclic alkyl moiety or to the alkyl portion of a larger substituent in which alkyl appears as its prefix root.

The terms “arylalkyl” and “alkylaryl” include an alkyl portion where alkyl is as defined above and to include an aryl portion where aryl is as defined above. Examples of arylalkyl include, but are not limited to, benzyl, fluorobenzyl, chlorobenzyl, phenylethyl, phenylpropyl, fluorophenylethyl, chlorophenylethyl, thienylmethyl, thienylethyl, and thienylpropyl. Examples of alkylaryl include, but are not limited to, toluene, ethylbenzene, propylbenzene, methylpyridine, ethylpyridine, propylpyridine and butylpyridine.

The term “heteroarylalkyl,” as used herein, shall refer to a system that includes an arylalkyl portion, where arylalkyl is as defined above, and contains one or two heteroatoms chosen from N, O or S.

The term “cycloarylalkyl,” as used herein, shall refer to a system that includes a 3- to 8-membered fully saturated cyclic ring portion and also includes an arylalkyl portion, where arylalkyl is as defined above.

In the compounds of the present invention, R¹ and R², when on the same carbon atom, can be taken together with the carbon atom to which they are attached to form a 3-6 membered ring.

In the compounds of the present invention, R^{a} and R^{b} can be taken together with any of the atoms to which they may be attached or are between them to form a 4-6 membered ring system.

The novel compounds of the present invention can be prepared according to the procedures of the following schemes and examples, using appropriate materials, and are further exemplified by the following specific examples. The compounds illustrated in the examples are not, however, to be construed as forming the only genus that is considered as the invention. The following examples further illustrate details for the preparation of the compounds of the present invention. Those skilled in the art will readily understand that known variations of the conditions and processes of the following preparative procedures can be used to prepare these compounds.

The compounds of the present invention can be prepared according to the general methods outlined in Schemes I-VI. CH₂R^{II} represents non-hydrogen values of R², or precursors thereof; R^{III} represents R³ or a precursor thereof; R^{IIIa} and R^{IIIb} represent non-hydrogen values of R³, or precursors thereof. R^{IV} represents OR^{a} and NR^{a}R^{b}. R^{O} represents an acyl group such as acetyl or the like; and R^{P} represents a N-protecting group for an indole, indazole, benzimidazole, or benzotriazole group.

### Scheme I

The fundamental methods for construction of 9a-substituted 1,2,9,9a-tertrahydro-3H-fluoren-3-one compounds are illustrated in Scheme L and are based on chemistry described by Cragoe, et al., J. Med. Chem. 1986, 29, 825-841. The indanone starting material (1a) of Scheme I is commercially available. A bromo- (chloro) substituent as R¹ can be introduced by reaction of the unsubstituted indonanone (1, R¹=H) with NBS (NCS). The 2-alkylidene-1-indanones (1b) are prepared by reacting 2-unsubstituted indanones (1a) with aldehydes under basic conditions. Reduction of the double bond (step 2) affords the indanone (1c). In step 3 of Scheme L a 2-substituted-1-indanone (1c) is reacted with a vinyl ketone in the presence of base. The crude product is then cyclized (step 3) under basic or acidic conditions. After O-deprotection the tetrahydrofluorenone products (1e) are obtained.

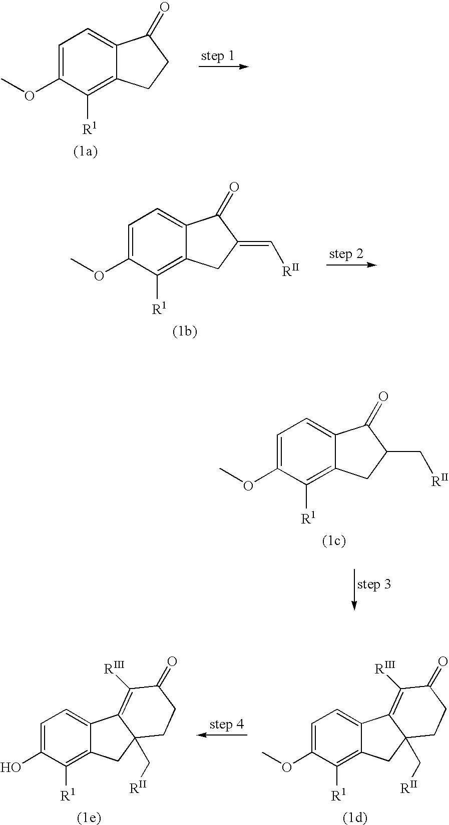

Representative reagents and reaction conditions indicated in Scheme I as steps 1-4 are as follows:

| | |
|---|---|
| Step 1 | R^{II} CHO, EtOH, KOH, rt |
| Step 2 | H₂, 10% Pd/C, EtOAc, rt |
| Step 3 | CH₂═CHC(O)CH₂R^{III}, DBN, THF, rt to 60° C. or |
| | CH₂═CHC(O)CH₂ R^{III}, NaOMe, MeOH, rt to 60° C., |
| | then pyrrolidine, HOAc, THF or PhMe, 60-85° C. or |
| | HOAc/6 N HCl, 80° C. |
| Step 4 | BBr₃, CH₂CL₂, −78° C. to rt |

### Scheme II

Tetrahydrofluorenones of types (1e) wherein R^{III} is hydrogen (2a) can be functionalized at the 4-position by the methods illustrated in Scheme II. Bromination (step 1) affords the 4-bromo intermediates (2b). These compounds can be converted (step 2) by known methods into a variety of new derivatives (2c) wherein R^{IIIb} is aryl, heteroaryl. If the group R^{IIIb} is or contains a functional group capable of further modification, this can be carried out to produce additional derivatives. For example, a R^{IIIb} 4-hydroxy-phenyl group can be alkylated at the oxygen.

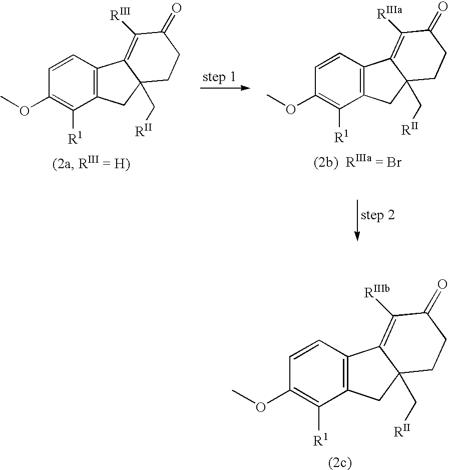

Representative reagents and reaction conditions indicated in Scheme II as steps 1 and 2 are as follows:

| | | |
|---|---|---|
| Step 1 | Br₂, NaHCO₃, CH₂Cl₂ or CCl₄, 0° C. to rt | R^{IIIa} = Br |
| Step 2 | R^{IIIb} SnBu₃, Pd(PPh₃)₄, PhMe, 100° C. or | R^{IIIb} = aryl, or |
| | | heteroaryl |

### Scheme III

Modifications to the C-3 ketone are outlined in Scheme III for the tetrahydrofluorenone derivative (3a). The methodology also applies to the other tetrahydrofluorenone products prepared according to Schemes I-II. In step 1, the ketone is reacted with a hydroxylamine or alkoxylamine reagent to yield the 3-imino product (3b).

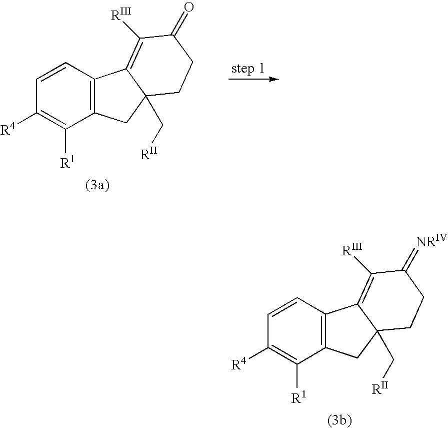

Representative reagents and reaction conditions indicated in Scheme III as step 1 are as follows:

Step 1 NH₂OR^{a}.HCl, pyridine, ETOH rt R^{IV}=OR^{a}

### Scheme IV

The principal method for constructing the tetracyclic 8,9,9a,10-tetrahydroindeno[2,1e]indazol-7(3H)-one compounds of the present invention is summarized in Scheme IV. Bromination (step 1) of 4-unsubstituted-5-acylamino)-1-indanones (4a) provides the 4-bromo compounds (4b) which can be converted (step 2) into the 4-methyl derivatives (4c) using Stille methodology. The 2-unsubstituted intermediates (4c) are converted to the corresponding 2-substituted compounds (4e) by an aldol condensation (step 3) and subsequent reduction (step 4). 4-Methyl-1-indanones (4c) reacts with vinyl ketones under basic conditions to provide diketones, which are then cyclized and deacylated under acidic or basic conditions (step 5) to afford the 7-amino-8-methyl-tetrahydrofluorenone intermediate (4f). If the cyclization is accomplished using pyrrolidine and acetic acid the amino group remains protected and allows a further elaboration before the formation of the fused pyrazole ring. Formation of the fused pyrazole ring is accomplished by treating (4f) with a diazotizing reagent followed by cyclization of the diazo intermediate with KOAc and dibenzo-8-crown-6 (step 6).

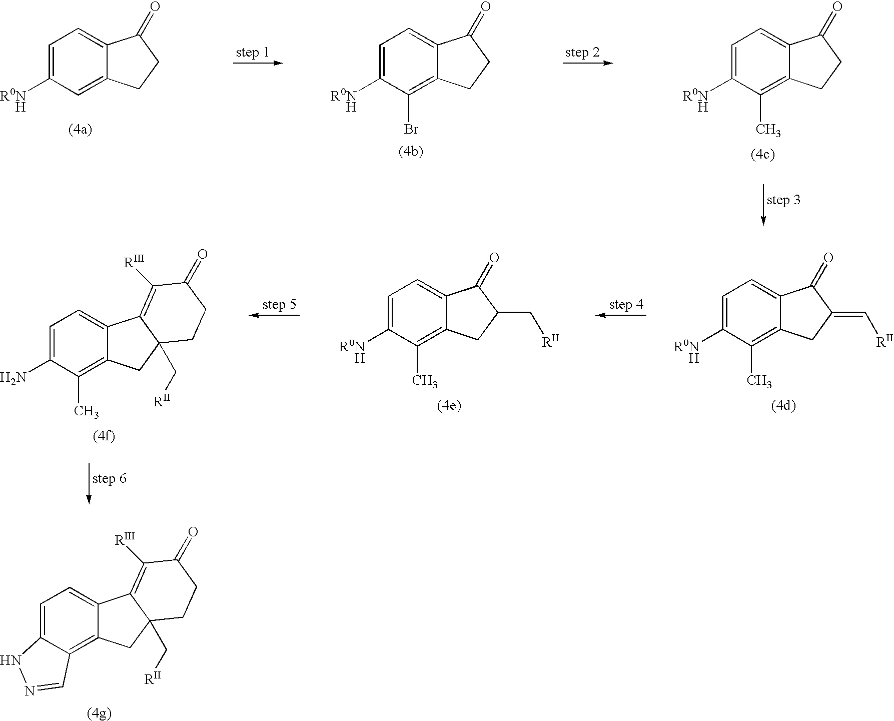

Representative reagents and reaction conditions indicated in Scheme IV as steps 1-6 are as follows:

| | |
|---|---|
| Step 1 | NBS, MeCN or DMF, 60° C. |
| Step 2 | Me₄Sn, PdCl₂(PPh₃)₂, PPh₃, LiCl, DMF, 100° C. |
| Step 3 | R^{II}CHO, MeOH, NaOMe, rt |
| Step 4 | H₂, 10% Pd/C, EtOAc, rt |
| Step 5 | CH₂═CHC(O)CH₂R^{III}, DBN, THF, rt to 60° C. or |
| | CH₂═CHC(O)CH₂ R^{III}, NaOMe, MeOH, rt to 60° C., |
| | then pyrrolidine, HOAc, THF or PhMe, 60-85° C. or |
| | HOAc, 6N HCl, 100° C. |
| Step 6 | i) NOBF₄, CH₂Cl₂, −45° C. to 10° C. |
| | ii) KOAc, dibenzo-18-crown-6, CH₂Cl₂, −40° C. to rt |

### Scheme V

Scheme V shows a method of synthesis of tetrahydroindeno[2,1-e]indazol-7(3H)-one compounds in which the R^{III} substituent is introduced onto a preformed tricyclic ring system. The 4-unsubstituted tetrahydrofluorenone intermediate (5a), which itself is prepared by cyclization (see Scheme IV, step 5) of intermediate (4c) wherein R^{III} is hydrogen, undergoes bromination (step 1) to afford the 4-bromo intermediates (5b). Deacylation (step 2), followed by pyrazole ring formation (step 3) affords the 6-bromo-tetrahydroindeno[2,1-e]indazol-7(3H)-one products (5d). The pyrazole group is N-protected (step 4) to give a mixture of 2- and 3-substituted derivatives (5e1) and (5e2) which can be used as such or which can be separated and used independently. The N-protected intermediates are converted by established methods (step 5) into a variety of new derivatives (5f) wherein R^{IIIb} is, inter alia, an alkyl, alkenyl, alkynyl, aryl, heteroaryl or arylalkyl group. Removal of the N-protection (step 6) affords the products (5g). If the group R^{IIIb} is, or contains, a functional group capable of further modification, such modifications can be carried out to produce additional derivatives. For example a 4-hydroxy-phenyl group can be alkylated at the oxygen.

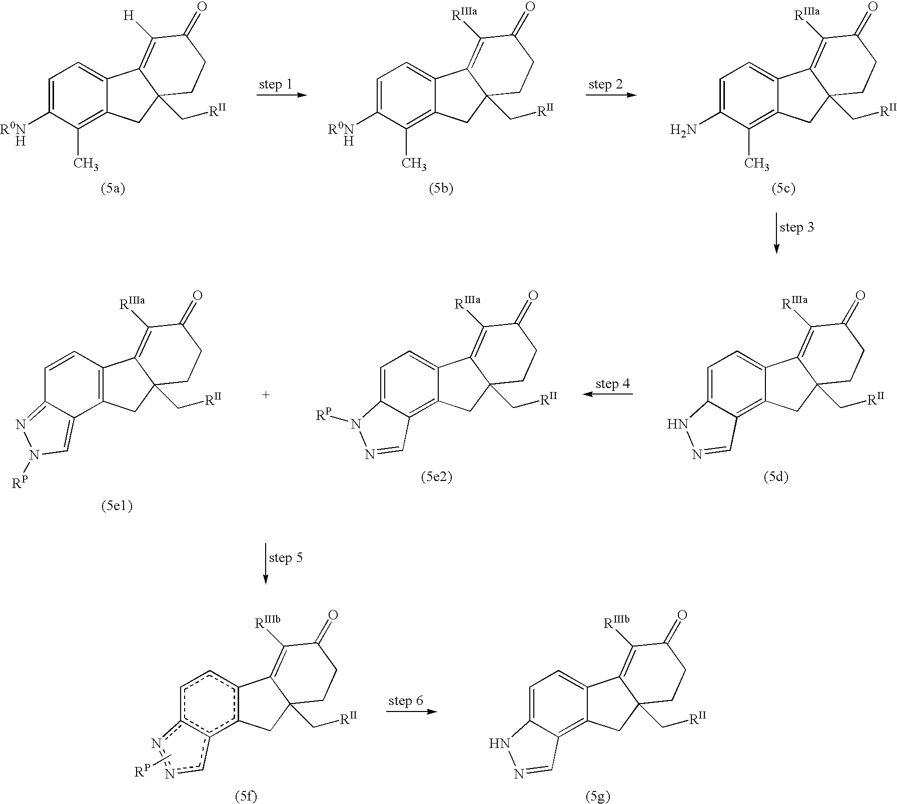

Representative reagents and reaction conditions indicated in Scheme V as steps 1-6 are as follows:

| | | |
|---|---|---|
| Step 1 | Br₂, NaHCO₃, CH₂Cl₂ or CCl₄, 0° C. to rt | R^{IIIa} = Br |
| Step 2 | NaOMe, MeOH and/or EtOH, rt to 80° C. | R^{O} = acetyl |
| Step 3 | i) NOBF₄, CH₂Cl₂, −45° C. to 10° C. | |
| | ii) KOAc, dibenzo-18-crown-6, CH₂Cl₂, −40° C. | |
| | to rt | |
| Step 4 | TsCl, DMAP, CH₂Cl₂, 0° C. to rt | R^{P} = Ts |
| Step 5 | R^{IIIb}SnBu₃, Pd(PPh₃)₄, PhMe, 100° C. | R^{IIIb} = |
| | | alkenyl, |
| | | aryl, or |
| | | heteroaryl |
| Step 6 | NaOH, 1,4-dioxane-EtOH, rt | R^{P} = Ts |

### Scheme VI

The principal method for constructing the 8,9,9a,10-tetrahydro-fluoreno[1,2-d][1,2,3]triazol-7(3H)-one compounds of the present invention is summarized in Scheme VI. After amino-protection of 5-amino-indane (step 1) bromination of unsubstituted 5-acylamino)-1-indane (6b) (step 2) provides the 6-bromo compound (7c) which is oxidized (step 3) to give the indanone-derivative (6d). After nitration and deprotection of the 5-amino group a catalytic reduction (step 6) leads to the diamino-intermediate (6g), which is subjected to cyclization (step 7) to form 7,8-dihydroindeno[4,5-d][1,2,3]triazol-6(3H)-one (6h). The 2-unsubstituted intermediate (6h) is converted to the corresponding 2-substituted compounds (6j) by an aldol condsation (step 8) and subsequent reduction (step 9). The 2-substituted 7,8-dihydroindeno[4,5-d][1,2,3]triazol-6(3H)-ones (6j) react with a vinyl ketone under basic conditions to provide diketones, which are then cyclized under acidic or basic conditions (step 10) to afford the 8,9,9a,10-tetrahydrofluoreno-[1,2-d][1,2,3]triazol-7(3H)-one derivatives (6k).

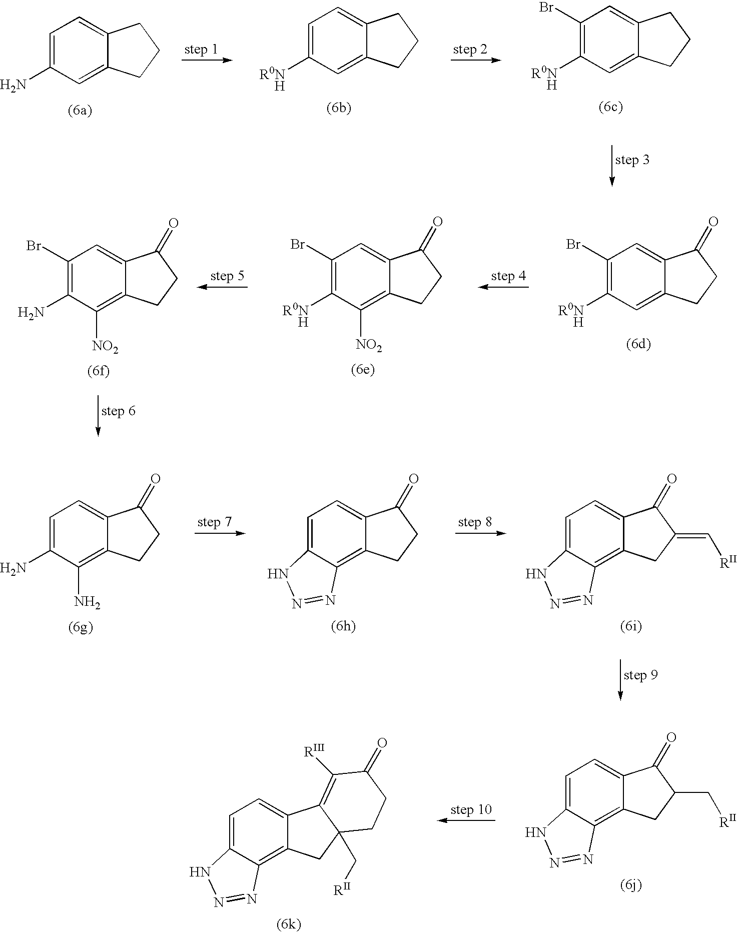

Representative reagents and reaction conditions indicated in Scheme VI as steps 1-10 are as follows:

| | | |
|---|---|---|
| Step 1 | Ac₂O, HOAc, reflux | R^{o} = Acetyl |
| Step 2 | Br₂, HOAc, 10° C. | |
| Step 3 | CrO₃, HOAC, 15° C. | |
| Step 4 | HNO₃, −40° C. | |
| Step 5 | MeOH, HCl, reflux | R^{o} = Acetyl |
| Step 6 | Pd(C), H₂, EtOAc, KOAc | |
| Step 7 | NaNO₂, HCl | |
| Step 8 | R^{II}CHO, MeOH, NaOMe, rt | |
| Step 9 | H₂, 10% Pd/C, EtOAc-EtOH, rt | |
| Step 10 | CH₂═CHC(O)CH₂R^{III}, DBN, THF, rt to | |
| | 60° C. or CH₂═CHC(O)CH₂ R^{III}, | |
| | NaOMe, MeOH, rt to 60° C., then pyrrolidine, | |
| | HOAc, THF or PhMe, 60-85° C. or | |
| | HOAc, 6N HCl, 100° C. | |

In Schemes I-VI, the various R groups often contain protected functional groups which are deblocked by conventional methods. The deblocking procedure can occur at the last step or at an intermediate stage in the synthetic sequence. For example, if one of R⁴ is a methoxyl group, it can be converted to a hydroxyl group by any of a number of methods. These include exposure to BBr₃ in CH₂Cl₂ at −78° C. to room temperature, heating with pyridine hydrochloride at 190-200° C., or treatment with EtSH and AlCl₃ in CH₂Cl₂ at 0° C. to room temperature. Another example involves the use of methoxymethyl (MOM) protection of alcohols and phenols. The MOM group is conveniently removed by exposure to hydrochloric acid in aqueous methanol. Other well-known protection-deprotection schemes can be used to prevent unwanted reactions of various functional groups contained in the various R substituents.

The specific examples I-III, while not limiting, serve to illustrate the methods of preparation of the 1,2,9,9a-tetrahydro-3H-fluoren-3-one compounds of the present invention. The specific examples IV-V, while not limiting, serve to illustrate the methods of preparation of the 8,9,9a,10-tetrahydroindeno[2,1-e]indazol-7(3H)-one compounds of the present invention. The specific example VI, while not limiting, serves to illustrate the methods of preparation of the 8,9,9a,10-tetrahydrofluoreno-[1,2-d][1,2,3]triazol-7(3H)-one compounds of the present invention. All compounds prepared are racemic, but could be resolved if desired using known methodologies.

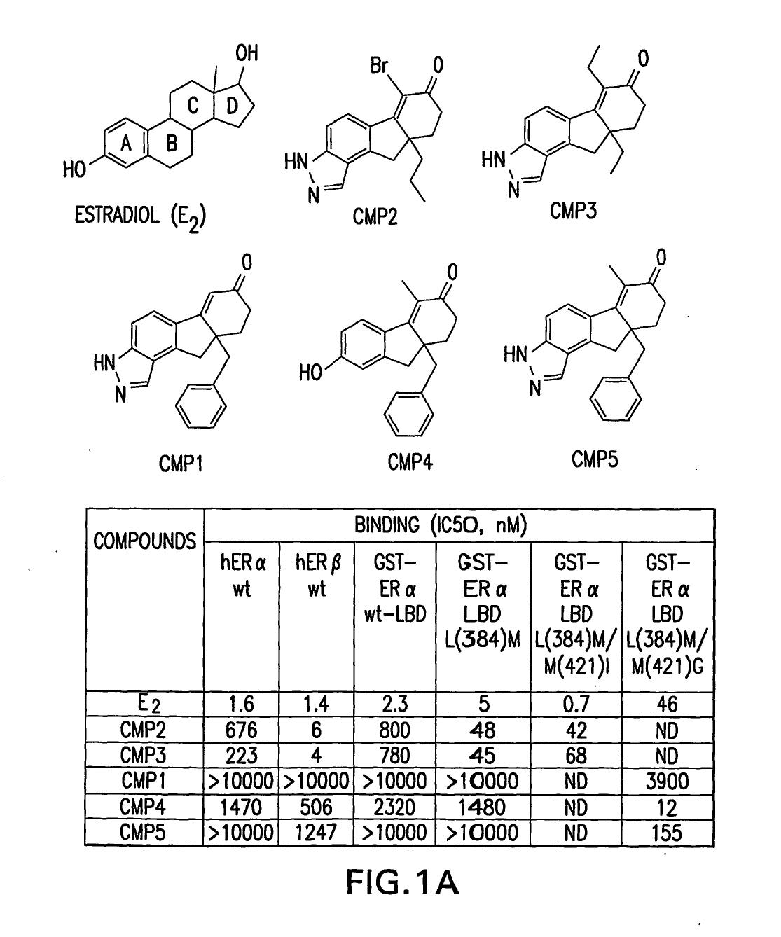

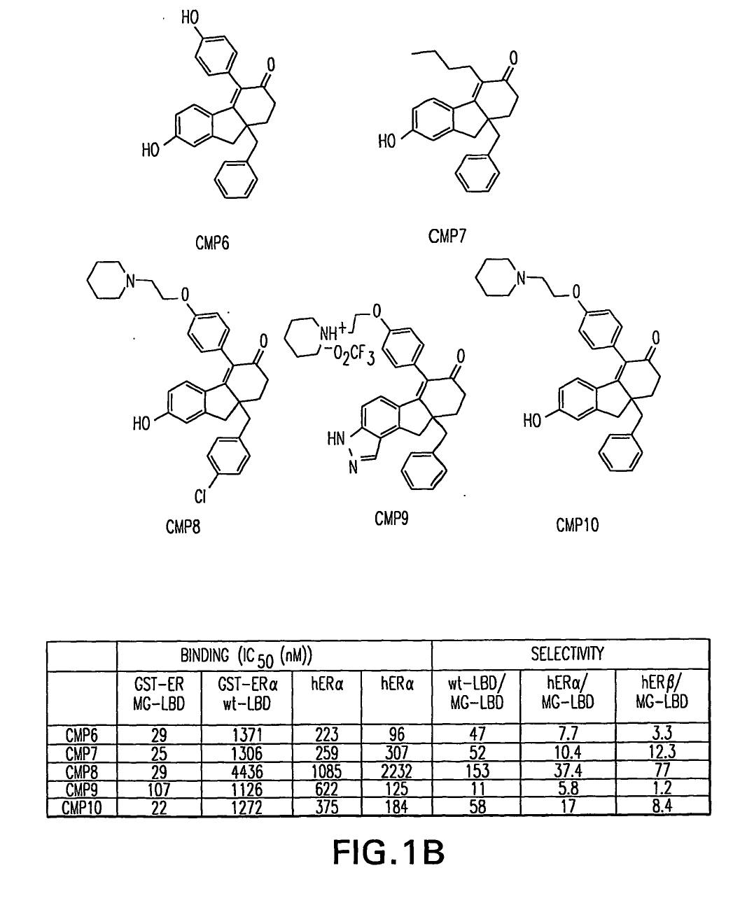

Using the methods disclosed above, various inactive analogues of estrogen, as well as active analogues of estrogen are synthesized, including but not limited to: CMP1, CMP2, CMP3, CMP4, CMP5 (FIG. 1A), CMP6, CMP7, CMP8, CMP9, CMP10 (FIG. 1B), and the compounds listed in tables 1a, 1b, 2 and 3.

### III. The Methods of Veneering the Ligand-binding Domains (LBDs) and Structures and Sequences of the Veneered LBDs

As discussed above, nuclear hormone receptors are modular proteins organized into structurally and functionally defined domains, including activation domain (AD), DNA-binding domain (DBD), and ligand-binding domain (LBD). Chimeric nuclear hormone receptors can be constructed by swapping the modular domains with their counterparts from other nuclear hormone receptors, or even proteins other than nuclear hormone receptors. The ligand-binding domain can subject the chimeric transcription factor to the control of the ligand binding to the domain. For instance, upon binding estrogen, the chimeric transcription factor with GAL4 DBD/ER LBD/VP16 AD is activated, binds to the binding site of GAL4, and promotes the transcription of the gene fused to the binding site.

The ligand-binding specificities of both nuclear hormone receptors and the chimeric transcription factors are determined by their LBD. The efforts of constructing an orthogonal gene switch system was therefore focused on the modification of the LBD. According to an embodiment of the present application, the veneered LBD for the chimeric transcription factors can be obtained with selected inactive analogues through the following approach.

First, mutations are introduced into the hydrophobic pocket region that surrounds the ligands to make the LBD be capable of binding the inactive analogues. The knowledge about the LBD and its ligands, and the structural differences between the ligands and the inactive analogues are used to guide the mutagenesis, leading to the production of mutant variants of the LBD. A library of transcription factors is constructed with the mutant variants fused to a DNA-binding domain (DBD), and an activation domain (AD).

Second, the library is screened with an inactive analogue to select the transcription factor that is capable of binding the inactive analogue and is activated by binding it. The selected transcription factors contain mutant variants of the LBD, which are the LBDs veneered to bind the inactive analogue.

Third, additional mutations are introduced into the selected veneered LBDs of the selected transcription factors, to decrease both their affinity for the natural ligand (hormone) and the ligand-independent activity of the transcription factors. The LBDs can then be used to construct the transcription factor appropriate for orthogonal gene switches, and the inactive analogues can be used as the exogenous ligands for the switches. Exogenous ligands for the switches can also be obtained by developing a new series of inactive analogues, based on the structures of the veneered LBDs.

According to a preferred embodiment of the present invention, the LBD of estrogen receptor (ER) was veneered, using the strategies disclosed above. The LBD is more preferrably from human estrogen receptor α (hERα).

According to a preferred embodiment of the present invention, a L384M mutation is introduced into hERα LBD. The L384M mutation is both necessary and sufficient to make hERα LBD have the binding specificities of hERβ LBD. Thus, the L384M hERα LBD can be veneered to bind the inactive analogues of hERβ ligands, such as CMP1. Five residues within the ligand-binding pocket of L384M hERα LBD were identified as the most likely candidate residues interfering with binding of CMP1: L391, F404, M421, I424, and L428.

A library of transcription factors is constructed containing mutant variants of L384M hERα LBD, whose L391, F404, M421, I424, and L428 are independently mutated into one of the following amino acid residues: Gly, Ala, Cys, Val, Ile, Leu, Met, Phe, Tyr and Trp. Each transcription factor in the library also contains a DNA-binding domain (DBD) and an activation domain (AD).

The library is then screened with an inactive analogue of a hERβ ligand, such as CMP1, to select transcription factors that can be activated by the inactive analogue. The selected transcription factors contain veneered LBDs that are capable of binding the inactive analogue. The amino acid sequences of the selected LBDs are provided in SEQ ID NO: 3-15.

According to a preferred embodiment of the present invention, mutations of D351A and H524V are introduced into the selected LBDs, as shown in SEQ ID NO: 3-15, to diminish their capability of binding the hormone and to reduce the ligand-independent activity of the transcription factor carrying the LBD. The amino acid sequences of the veneered hERα LBDs are provided in SEQ ID NO: 16-28.

The veneered LBDs of SEQ ID NO: 16-28 are capable of binding certain inactive analogues of hERβ ligands, such as CMP1, CMP4, CMP5, and CMP 11-38 (FIG. 1A and tables 1-3), and to be activated by the binding, while the LBDs do not bind the natural hER ligand.

Alternatively, a G521R mutation is introduced into the selected LBDs, as shown in SEQ ID NO: 3-15, to diminish their capability of binding the natural hormone and to reduce the ligand-independent activity of the transcription factor carrying the LBD. The amino acid sequences of the veneered hERα LBDs are provided in SEQ ID NO: 29-41.

A new series of inactive analogues of hERβ ligands, including CMP6, CMP7, CMP8, CMP9, and CMP10 (FIG. 1B), are synthesized to fit to the LBDs carrying the G521R mutation.

The veneered LBDs of SEQ ID NO: 29-41 are capable of binding certain inactive analogues of hERβ ligands, such as CMP6, CMP7, CMP8, CMP9, and CMP10, and to be activated by the binding, while they do not bind the natural hER ligand.

The present invention also provides polynucleotides that encode the veneered hERα LBDs as shown in SEQ ID NO: 1-41.

### IV. Chimeric Transcription Factors Containing the Veneered LBDs

The present invention provides a novel chimeric transcription factor. The chimeric transcription factor is activated or repressed by an inactive analogue of the naturally occurring ligand of the nuclear hormone receptor, while it does not respond to the naturally occurring ligand itself.

According to an embodiment of the present invention, the chimeric transcription factor comprises the veneered ligand-binding domain (LBD) of a nuclear hormone receptor, a transcriptional regulatory domain, which can be an activation domain (AD) or a repression domain, and a DNA-binding domain (DBD).

It should be noted that the three essential components of the ligand binding-dependent transcription factors, namely the DNA-binding domain, the ligand-binding domain and the transcriptional regulatory domain, may be arranged in any order or sequence in a transactivator/transrepressor fusion protein of the invention.

According to a preferred embodiment of the present invention, all the domains of the chimeric transcription factor are from, or veneered from, proteins of human origin.

Gene expression in cells of non-human mammal is often desired. According to an alternative embodiment of the present invention, the domains of animal origin rather than human origin can be used. The invention therefore further pertains to any chimeric transcription factor that comprises the domains of mammalian species other than human, including rabbit, guinea pig, rat, mouse or other rodent, cat, dog, pig, sheep, goat, cattle or horse, or which is a bird, such as a chicken.

1. The Veneered Ligand-Binding Domain (LBD)

According to an embodiment of the present invention, the chimeric transcription factor comprises the veneered ligand-binding domain (LBD) of a nuclear hormone receptor. The veneered ligand-binding domain does not bind the naturally occurring ligand of the nuclear hormone receptor, but binds an inactive analogue of the naturally occurring ligand. Binding of the inactive analogue to the veneered ligand-binding domain activates the transcription factor. The inactive analogue neither binds nor activates the nuclear hormone receptor.

According to a preferred embodiment of the present application, the novel transcription factor comprises a veneered ligand-binding domain (LBD) of human estrogen receptor α (hERα).

According to a further preferred embodiment of the present application, the novel transcription factor comprises a veneered ligand-binding domain having a sequence selected from the group consisting of SEQ ID NO: 16-41.

2. The Transcriptional Regulatory Domain

The transcriptional regulatory domain of the chimeric transcription factor may be any available to those skilled in the art. The transcriptional regulatory domain can be an activation domain (AD). Polypeptides that activate transcription in eukaryotic cells are well known in the art. In particular, transcriptional activation domains of many DNA binding proteins have been described and have been shown to retain their activation function when the domain is transferred to a heterologous protein.

Transcriptional activation domains found within various proteins have been grouped into categories based upon similar structural features. Types of transcriptional activation domains include acidic domains, proline-rich domains, serine/threonine-rich domains and glutamine-rich domains. Examples of the acidic domains include the VP16 regions already described and amino acid residues 753-881 of GAL4. Examples of the proline-rich domains include amino acid residues 399-499 of CTF/NFI and amino acid residues 31-76 of AP2. Examples of the serine/threonine-rich domains include amino acid residues 1-427 of ITF1 and amino acid residues 2-451 of ITF2. Examples of the glutamine-rich domains include amino acid residues 175-269 of Oct1 and amino acid residues 132-243 of Spl. The amino acid sequences of each of the regions described above, and of other useful transcriptional activation domains, are disclosed in Seipel, K. et al. (EMBO J., 1992 12:4961-4968).

In a preferred embodiment of the present invention, the activation domain is an activation domain (AD) of human p65 protein (Schmitz, M. L. and Bauerle, P. A., 1991, EMBO J., 10:3805-3817), more preferably comprising the region spanning amino acids 285-551 of human p65, or a transcription-activating portion encompassed within this region. In another embodiment, multimers of the p65 AD may be used. In another embodiment, multimers of portions of the p65 AD may be used.

In another preferred embodiment of the present invention, the activation domain comprises the herpes simplex virus virion protein 16 (VP16) (Triezenberg, S. J. et al. (1988) Genes Dev. 2:718-729). Preferably, about 127 of the C-terminal amino acids of VP16 are used; more preferably, about 11 of the C-terminal amino acids (amino acids 437-447) of VP16 are used. Preferably, multimers (two to four monomers) of this region are used; more preferably, a dimer of this region (i.e., about 22 amino acids) is used. Suitable C-terminal peptide portions of VP16 are described in Seipel, K. et al. (EMBO J., 1992 13:4961-4968). For example, a dimer of a peptide having an amino acid sequence DALDDFDLDML can be used.

In another embodiment of the present invention, the activation domain comprises or consists of the AD of the PPARy-1 coactivator (PGC-1) (Puigserver P. et al., 1998, Cell, 92, 829). In one embodiment, the region spanning aa 1-70 of the N-terminus of PGC-1 is used (Puigserver, P., Science, 1999, 1368-1371). In another embodiment, the region spanning aa 1-65 of the N-terminus of PGC-1 is used. In another embodiment, multimers of the PGC-1 AD, or portions of it, may be used.

In another embodiment, transcription is activated by an indirect mechanism, through recruitment of a transcriptional activation protein to interact with a fusion protein comprising DBD and regulatory domain. This may, for example, be via a polypeptide domain (e.g., a dimerization domain) which mediates a protein-protein interaction with a transcriptional activator protein, such as an endogenous activator present in a host cell.

Other polypeptides with transcriptional activation ability in eukaryotic cells can also be used in an activation domain in accordance with the present invention.

Moreover, the transcriptional regulatory domain can also be a repression domain. In other embodiments, chimeric transcription factors capable of repressing transcription are generated (Transcriptional Repressors). In this case, the transcription factor comprises a repression domain, which directly or indirectly repress transcription in eukaryotic cells.

Polypeptides that repress transcription in eukaryotic cells are well known in the art. In particular, transcriptional repression domains of many DNA binding proteins have been described and have been shown to retain their activation function when the domain is transferred to a heterologous protein (Deuschle et al., 1995, Mol. Cell. Biol. 15, 1907-1914; Freundlieb S. et al., 1999, J. Gene Medicine, 1, 1).

An example of such domains, capable of repressing instead of activating transcription, is the KRAB repressor domain of the human Koxl zinc finger protein (Margolin J., 1994, Proc. Natl. Acad. Sci. USA, 91, 4509-4513). This domain can be used either as single domain or in multimeric forms.

3. The DNA-Binding Domain (DBD)

The DNA-binding domain of the chimeric transcription factor may be any available to those skilled in the art. Polypeptides that bind to DNA in eukaryotic and prokaryotic cells are well known in the art. In particular, DNA-binding domains of many DNA binding proteins have been described and have been shown to retain their DNA-binding function when the domain is transferred to a heterologous protein.

DNA-binding domains found within various proteins have been grouped into categories based upon similar structural features. Types of DNA-binding domains include those with helix-turn-helix motif, zinc finger motif (Frankel, A. D. et al. (1988) Science 240:70-73), leucine zipper motif (Landschulz et al. (1989) Science 243:1681-1688), or helix-loop-helix motif (Murre, C. et al. (1989) Cell 58:537-544), and those from high mobility group. The helix-turn-helix motif is a component of homeobox domain, which has been identified in many invertebrate and vertebrate regulators of gene expression. Zinc-finger motifs have been identified in TIIIFA, and steroid hormone receptors. The leucine zipper motif has been found in the proto-oncoprotein Myc, Fos and Jun. The helix-loop-helix motif has been found in myogenic transcription factors. In addition to DNA-binding, the domains containing zinc finger motif, leucine zipper motif, or helix-loop-helix motif also play a role in the dimerization of transcription factors.

According to a preferred embodiment of the present invention, the DNA-binding domain is from a tissue-specific transcription factor, which is expressed in tissues other than the tissues that the chimeric transcription factor is desired to be exprerssed.

According to a preferred embodiment of the present invention, the DNA-binding domain is a DNA-binding domain of human HNF-1. Chimeric transcription factors containing the DBD specifically activate (or repress) transcription of sequences controlled by HNF-1 responsive promoters. Chimeric transcription factors containing the HNF-1 DBD are useful for regulating, in tissues that do not express endogenous HNF-1, the level of transcription of any target gene linked to the selected HNF-1 DNA binding sites.

HNF-1 (also called LF-B1 or HNF-1α) is a transcription factor that has been implicated as a major determinant of hepatocyte-specific transcription of several genes (Frain M. 1990, Cell, 59, 145-157). The consensus binding site derived from these sequences is the palindrome GGTTAAT(N)ATTAATA (SEQ ID NO: 42) (Tronche F. et al., 1997, J. Mol. Biol., 266:231-245). Consistent with the dyad symmetry of this site, HNF-1 binds DNA as a dimer. The DNA binding domain is located in the first N-terminal 281 aa of HNF-1 (DBD=1-281).

Natural HNF-1 polypeptides are expressed at high levels in hepatocytes. They are also expressed in tissues other than liver, such as kidney, intestine, stomach and pancreas. However, HNF-1 proteins are not naturally expressed in several cell lines and tissues, such as muscle.

A transgene cloned downstream of an HNF-1-dependent promoter is not transcribed when delivered in cells lacking endogenous HNF-1 (Toniatti C. et al., 1990, EMBO J., 9, 4467-4475). Since HNF-1 is not present in muscles, a transgene cloned downstream of an HNF-1-dependent promoter may be silent when delivered into muscle cells in vivo and in vitro. However, previous results obtained in vitro provide indication that such a transgene could be activated if an expression vector encoding for HNF-1 is co-delivered into muscles (Toniatti C. et al., 1990, EMBO J., 9, 4467-4475).

In a preferred embodiment of the present invention, the HNF-1 DNA binding domain comprises or consists of residues 1-282 of human HNF-1 (Bach, et al (1990), Genomics, 8(1):155-164 (Sequence accession number P20823), or a DNA-binding portion encompassed within these residues.

The present invention provides a transcription factor that comprises SEQ ID NO: 43. The present invention also provides a polynucleotide that encodes the transcription factor as shown in SEQ ID NO: 43.

According to an alternative embodiment of the present invention, the DBD is GAL4 minimal DBD.

Other polypeptides with DNA-binding ability in eukaryotic and prokaryotic cells can also be used in a DNA-binding domain in accordance with the present invention.

4. Other Domains of the Chimeric Transcription Factor

A chimeric transcription factor of the present invention (which may be a single fusion protein) may further comprise one or more additional polypeptide components, such as a nuclear localization signal (NLS), which promotes transport into a cell nucleus.

Nuclear localization signals typically are composed of a stretch of basic amino acids. When attached to a heterologous protein (e.g., a fusion protein of the invention), the nuclear localization signal promotes transport of the protein to a cell nucleus. The nuclear localization signal is attached to a heterologous protein such that it is exposed on the protein surface and does not interfere with the function of the protein. Preferably, the NLS is attached to one end of the protein, e.g. the N-terminus. The amino acid sequence of a non-limiting example of an NLS that can be included in a fusion protein of the invention is Met-Pro-Lys-Arg-Pro-Arg-Pro (SEQ D NO: 44). Preferably, a nucleic acid encoding the nuclear localization signal is spliced by standard recombinant DNA techniques in-frame to the nucleic acid encoding the fusion protein (e.g., at the 5′ end).

### V. Orthogonal Gene Switches Using the Chimeric Transcription Factor and the Inactive Analogues

The present invention further provides an orthogonal gene switch for regulating the expression of a desired gene. The gene switch comprises a novel chimeric transcription factor which, as discussed above, is activated or repressed by an inactive analogue of the naturally occurring ligand of the nuclear hormone receptor, but does not respond to the naturally occurring ligand itself.

The present invention therefore provides an orthogonal gene switch that is capable of controlling the expression of a heterologous gene or a series of heterologous genes through the application of an effective exogenous ligand. One of the major advantages of the present invention is that there is no mutual interference between the gene switches and endogenous gene regulation systems. In other words, the exogenous inducer cannot activate the endogenous gene expression, while the endogenous hormones cannot turn on the gene switch.

According to an embodiment of the present invention, the orthogonal gene switch also comprises a construct comprising a desired gene, and a regulatory region that is fused to the desired gene. The transcription factor is capable of binding to the regulatory region.

According to a preferred embodiment of the present invention, the gene switch is modulated by an exogenous ligand, which is the inactive analogue. Thus, the exogenous ligand can be used to control the expression of the desired gene, without interferring the functions of endogenous nuclear hormone receptor.

1. The Gene Desired to be Regulated

The gene desired to be regulated is a nucleotide sequence of interest whose transcription is regulated by the gene switch. According to an embodiment of the present invention, the desired gene encodes a polypeptide or peptide, an antisense sequence, a dsRNA (double-stranded RNA), an siRNA (short interfering RNA), or a ribozyme.

A polypeptide whose expression may be controlled using the present invention may be selected according to the desires and aims of the person performing the invention, and may be a therapeutic protein or a cytotoxic protein. The type of the therapeutic protein is determined by the disease to be treated. For instance, The therapeutic protein used in cancer gene therapy can be cytokines (Agha-Mohammadi, S. and Lotze, M. T., J. Clin. Invest. 105, 1173-1176 (2000)), prodrug activating enzymes (Springer, C. J. and Niculescu-Duvaz, I., J. Clin. Invest. 105, 1161-1167 (2000)), antibodies, and tumoricidal gene products.

Polypeptide expression may be inhibited by using appropriate nucleic acid to influence expression by antisense regulation, and an antisense sequence may be placed under transcriptional control in accordance with the present invention. The use of anti-sense genes or partial gene sequences to down-regulate gene expression is now well-established. Double-stranded DNA is placed under the control of a promoter in a “reverse orientation” such that transcription of the “anti-sense” strand of the DNA yields RNA which is complementary to normal mRNA transcribed from the “sense” strand of the target gene. The complementary anti-sense RNA sequence is thought then to bind with mRNA to form a duplex, inhibiting translation of the endogenous mRNA from the target gene into protein. Whether or not this is the actual mode of action is still uncertain. However, it is established fact that the technique works.

Alternatively, gene expression can also be inhibited by RNAi (RNA interference), and a sequence coding a dsRNA or an siRNA may be placed under transcriptional control in accordance with the present invention. Double-stranded RNAs are capable of inhibiting gene expression in a sequence-specific manner in diverse organisms, such as plant, C. elegans, and Drosophilia (Hommond S. M., Nature Review Genetics 2, 110-119 (2001)). Moreover, siRNA, which is short dsRNA, has been shown to prohibit gene expression in a sequence-specific manner by causing RNAi (Elbashir, S. M., et al., Nature 411, 494-498 (2001); Bass, B. L., Nature 411, 428-429 (2001)).

SiRNA can be continually produced in transfected mammalian cells, by the expression of small hairpin RNAs (shRNAs), which are processed into siRNA by the RNA machinaery in vivo (Brummelamp T. R., et al., Science 296, 550-553 (2002); Paddison, P. J., et al., Genes & Dev. 16, 948-958 (2002); Paul, C. P., et al., Nature Biotechnol. 20, 505-508 (2002)). Alternatively, SiRNA can be produced in mammalian cells by the expression of both sense RNA and antisense RNA, which then hybridize in vivo to form siRNA (Miyagishi, M., and Taira K., Nature Biotechnol. 20, 497-500 (2002)). The small hairpin RNA, or the sense RNA and antisense RNA, can be placed under the control of a promoter responsive to a chimeric transcription factor according to the present invention.

Another possibility is that nucleic acid is used which on transcription produces a ribozyme, able to cut nucleic acid at a specific site—thus also useful in influencing gene expression. Background references for ribozymes include Kashani-Sabet and Scanlon (1995). Cancer Gene Therapy, 2, (3) 213-223, and Mercola and Cohen (1995). Cancer Gene Therapy 2, (1) 47-59.

2. The Regulatory Region

The desired gene is operatively linked to the regulatory region containing at least one oligonucleotide sequence to which the chimeric transcriptional factor binds. The regulatory region is usually located upstream (i.e., 5′) to the sequence to be transcribed and, where appropriate, minimal promoter. The regulatory region sequence may also be operatively linked downstream (i.e., 3′) of the nucleotide sequence to be transcribed.

According to an embodiment of the present invention, the regulatory region may comprise single or mutimeric binding sites of the chimeric transcription factor. According to a preferred embodiment of the present invention, the regulatory region is an artificial promoter that is controlled by the chimeric transcription factor. The artificial promoter comprises one or multiple binding sites of the chimeric transcription factor.

According to an embodiment of the present invention, the chimeric transcription factor comprises the DNA-binding domain of HNF-1; the promoter responsive to the transcription factor may comprise at least one binding site of HNF-1 and one or more binding sites for one or more different transcription factors.

3. The Exogenous Ligand

The present invention provides exogenous ligands that are capable of binding to and activating the chimeric transcription factor, but incapable of binding the corresponding wild-type nuclear hormone receptor. The exogenous ligands are preferably inactive analogues of antagonists or agonists of the wt nuclear hormone receptor.

According to an embodiment of the present invention, the exogenous ligand is an inactive analogue of a human estrogen receptor β (hERβ)-specific agonist or antagonist. According to a preferred embodiment of the present invention, the inactive analogue is selected from the group consisting of: CMP1, CMP4, CMP5, CMP6, CMP7, CMP8, CMP9, CMP10, and the compounds listed in tables 1a, 1b, 2 and 3. The Structures and Syntheses of the compounds have been discussed above.

According to a preferred embodiment of the present application, the orthogonal gene switch comprises a chimeric transcription factor having a LBD whose amino acid sequence is selected from the group consisting of SEQ ID NO: 16-41.

For the gene switches comprising an amino acid sequence selected from the group consisting of SEQ ID NO: 16-28, the exogenous ligand is selected from the group consisting of: CMP1, CMP4, CMP5, and CMP11-38.

For the gene switches comprising an amino acid sequence selected from the group consisting of SEQ ID NO: 2941, the exogenous ligand is selected from the group consisting of: CMP6, CMP7, CMP8, CMP9, and CMP10.

Expression of the sequence of interest in target cells is stimulated by administering the exogenous ligand to the target host cell. To stop expression of the gene of interest in cells of the subject, administration of the exogenous ligand is stopped.

Where a repression domain is employed in the chimeric transcription factor, expression of the sequence of interest in target cells is repressed in the presence of the ligand and then stimulated by its withdrawal. To stop expression of the gene of interest in cells of the subject, the ligand is readministered.

In both cases the level of gene expression can be modulated by adjusting the dose of the ligand administered to the patient. Thus, in a host cell, transcription of the desired gene may be controlled by altering the concentration of the exogenous ligand in contact with the host cell (e.g. adding the ligand to a culture medium, or administering the ligand to a host organism, etc.).

### VI. The Applications of the Orthogonal Gene Switch System

Heterologous proteins are expressed for various purposes in genetically engineered eukaryotic cells such as yeast cells and mammalian cells. A gene switch according to the present invention can be used to regulate the expression of the heterologous proteins in the eucaryotic cells. In addition, the switch provides a further advantage in eukaryotic cells where accumulation of large quantities of a heterologous protein can damage the cells, or where the heterologous protein is damaging such that expression for short periods of time is required in order to maintain the viability of the cells.

An orthongonal gene switch according to the present invention can be widely applicable to a variety of situations where it is desirable to be able to regulate gene expression in host cells such as cultured eukaryotic cells.

Such an inducible system also has applicability in gene therapy allowing the timing of expression of the therapeutic protein to be controlled. The present invention is therefore not only applicable to transformed mammalian cells but also to mammals per se.

Expression of the gene of interest in host cells is stimulated or repressed by administering the exogenous ligand to the patient, or to the cells directly. To stop expression of the gene of interest, administration of the exogenous ligand is stopped.

1. Gene Therapy

The invention is preferentially employed for gene therapy purposes in humans and/or for research purposes in non-human species. Gene therapy is the treatment of certain disorders, especially those caused by genetic anomalies or deficiencies, by introducing specific engineered genes into a patient's cells. Gene therapy has been developed to treat various diseases. The candidate diseases for gene therapy include cancer, cardiovascular disease, cystic fibrosis, AIDS, Gaucher's disease, familial hypercholesterolemia, rheumatoid arthritis and sickle cell anemia, and muscular dystrophy.

Regulatable gene expression is often crucial for gene therapy. The regulation of gene expression can be achieved with an orthorgonal gene switch according to the present invention. Cells of a subject in need of gene therapy may be modified to contain (1) nucleic acid encoding the chimeric transcription factor in a form suitable for expression in the host cells, and (2) a sequence of interest (e.g. for therapeutic purposes) operatively linked to a promoter responsive to the chimeric transcription factor.

According to an alternative embodiment of the present invention, the orthogonal gene switch is used in veterinary gene therapy. The invention therefore further pertains to gene therapy in mammalian species other than human. The non-human mammalian species can be selected from the group consisting of rabbit, guinea pig, rat, mouse, cat, dog, pig, sheep, goat, cattle and horse. Alternatively, the invention pertains to gene therapy in avian species, such as a chicken.

In addition to regulatable gene expression, effective gene therapy requires efficient delivery of the gene switch to targeted mammalian cells.

Gene therapy can also be used to treat diseases in combination with other therapies (W. M. Rideout III et al., Cell 109, 17-27 (2002)).

To induce or repress transcription in vivo, the ligand may be administered to the body, or a tissue of interest (e.g. by injection). The ligand should be non-toxic to the body. The body to be treated may be that of an animal, particularly a mammal, which may be human or non-human. Suitable routes of administration include oral, intraperitoneal, intramuscular, i.v. The ligand can then be absorbed by the target cells. In all cases described, the concentration of the ligand will be proportional to the concentration of chimeric transcription factor expressed in the host cells.

2. Other Uses of the Orthogonal Gene Switch

Besides the use for gene therapy outlined in the previous sections, orthogonal gene switches according to the present invention can be used to:

1) conditionally express a suicide gene in cells, thereby allowing for elimination of the cells after they have served an intended function. For example, cells used for vaccination can be eliminated in a subject after an immune response has been generated by the subject by inducing expression of a suicide gene in the cells with the specific ligand.

2) modulate expression of genes that are contained in recombinant viral vectors and might interfere with the growth of the viruses in the packaging cell lines during the production processes. These recombinant viruses might be derivatives of Adenoviruses, Retroviruses, Lentiviruses, Herpesviruses, Adenoassociated viruses, and other viruses that are familiar and obvious to those skilled in the art.

3) provide large-scale production of a toxic protein of interest using cultured cells in vitro that do not contain endogenous active transcription factors that bind the binding site. The cultured cells have been modified to contain a nucleic acid encoding the chimeric transcription factor in a form suitable for expression of the transcription factor in the cells and a gene encoding the protein of interest operatively linked to a promoter responsive to the transcription factor.

4) determine whether the functions of a gene. The expression of an endogenous gene can be changed by the orthogonal gene switch. For instance, a chimeric transcription factor can be used to control the production of an SiRNA to suppress the expression of the target gene, and determine the functions of the gene according to the phenotype caused by the suppression.

One convenient way of producing a polypeptide or fusion protein according to the present invention is to express nucleic acid encoding it, by use of nucleic acid in an expression system.

Accordingly, the present invention also provides in various aspects nucleic acid encoding the transcriptional activator or repressor of the invention, which may be used for production of the encoded protein.

3. Delivery of the Gene Switch to the Host Cells

To be effectively delivered to the target cells, gene switches according to the present invention can be carried by appropriate vectors. Suitable vectors can be chosen or constructed, containing appropriate regulatory sequences, including promoter and/or enhancer sequences responsive to a chimeric transcription factor according to the present invention, terminator fragments, polyadenylation sequences, sequences, marker genes, and other sequences as appropriate. Moreover, tissue-specific regulatory elements can also be used in the vectors, to regulate expression of a polypeptide or fusion protein preferentially in a particular cell type.

For further details of the DNA recombinant techniques used in vector construction, see, for example, Molecular Cloning: a Laboratory Manual: 3rd edition, Sambrook et al., 2001, Cold Spring Harbor Laboratory Press. Many known techniques and protocols for manipulation of nucleic acid, for example in preparation of nucleic acid constructs, mutagenesis, sequencing, introduction of DNA into cells and gene expression, and analysis of proteins, are described in detail in Current Protocols in Molecular Biology, Ausubel et al. eds., John Wiley & Sons, 2003.

In gene therapy, the nucleic acid according to the present invention can be introduced into the host cells via either in vivo approach or ex vivo approach. Under the in vivo approach, nucleic acid is directly delivered to the target cells in the subject to be treated. Under the ex vivo approach, target cells are first taken from the treated subject, transfected with the nucleic acid in vitro, and are then implanted or otherwise administered back to the treated host.

For both in vivo and ex vivo gene delivery, appropriate vectors include plasmid, viral vector, and liposome. According to an embodiment of the present invention, the recombinant expression vector is a plasmid.

In a preferred embodiment of the present invention, the recombinant expression vector is a veneered virus, or portion thereof, which allows for expression of a nucleic acid introduced into the viral nucleic acid. For example, replication defective retroviruses, adenoviruses and adeno-associated viruses can be used. The genome of a virus such as adenovirus can be manipulated such that it encodes and expresses a chimeric transcription factor according to the present invention, but is inactivated in terms of its ability to replicate in a normal lytic viral life cycle. Protocols for producing recombinant retroviruses and for infecting cells in vitro or in vivo with such viruses can be found in Ausubel, et al. (supra).

Vectors such as viral vectors have been used to introduce nucleic acid into a wide variety of different target cells. Typically the vectors are exposed to the target cells so that transfection can take place in a sufficient proportion of the cells to provide a useful therapeutic or prophylactic effect from the expression of the desired polypeptide.

A variety of vectors, both viral vectors and plasmid vectors, are known in the art (see, e.g., U.S. Pat. No. 5,252,479 and WO 93/07282). In particular, a number of viruses have been used as gene transfer vectors, including papovaviruses, such as SV40, vaccinia virus, herpesviruses, including HSV and EBV, retroviruses, adenoviruses, and adeno-associated viruses.

The nucleic acid may be on an extra-chromosomal vector within the cell, or otherwise identifiably heterologous or foreign to the cell. An example of extra-chromosomal vectors is adenoviral vector. Adenovirus can infect a broad range of human cells, including those of the lung, liver, blood vessels and brain, but cannot integrate into the genome of the host cell. The treatment using an extra-chromosomal vector may have to be repeated periodically.

Alternatively, the transfected nucleic acid may be permanently incorporated into the genome of each of the targeted cells, providing long lasting effect. Adeno-associated virus, retrovirus, including oncoretrovirus and lentivirus, are also capable of infecting various human cells, and integrate into the genome of the host cell. Many gene therapy protocols in the prior art have used disabled murine retroviruses, and more recently, lentiviruses. Lentivirual vectors, like oncoretroviral vectors, can integrate into the genome of nondividing cells, such as hematopoietic stem cells in their primitive state. Viral vectors derived from these viruses can therefore be constructed as integrating vector.

Integration may also be promoted by inclusion of sequences that promote recombination with the genome, in accordance with standard techniques. Recombinant adenoviruses have been among the most widely explored vector systems for delivering genes to mammalian cells. However, it is difficult to achieve long-term gene expression using recombinant adenoviral vectors, because adenovirus is incapable of integrating into the genome of host cells. One solution to this problem is the recombinant adenovirus carrying “Sleeping Beauty” transposon machinery and Flp recombinase. The recombinant virus can integrate the desired transgene into the genome of the host cells (Yant, S., Nat. Biotechnol. 20, 999-1005 (2002)).

As an alternative to the use of viral vectors, other known methods of introducing nucleic acid into cells in gene therapy includes transfer mediated by liposomes and direct DNA uptake and receptor-mediated DNA transfer. Receptor-mediated gene transfer, in which the nucleic acid is linked to a protein ligand via polylysine, with the ligand being specific for a receptor present on the surface of the target cells, is an example of a technique for specifically targeting nucleic acid to particular cells.

The nucleic acids need to be delivered to the host cells can include, a first nucleic acid encoding a chimeric transcription factor as disclosed, and a second nucleic acid comprising a nucleotide sequence to be transcribed operatively linked to a transcription unit.

In one embodiment, the first and second nucleic acids are separate molecules, i.e., in two different vectors. In this case, a host cell may be cotransfected with the two vectors or successively transfected with the vectors. In another embodiment, the nucleic acids are linked (i.e., colinear) in the same molecule, i.e., in a single vector. In this case, a host cell may be transfected with the single nucleic acid molecule, which is preferred in gene therapy.

In an alternative, a sequence to be transcribed may be endogenous to a host cell. An endogenous sequence may be operatively linked to an appropriate transcription unit by means of homologous recombination. For example, a homologous recombination vector can be prepared which includes a promoter sequence responsive to a chimeric transcription factor of the present invention, flanked at its 3′ end by sequences representing the coding region of the endogenous gene and flanked at its 5′ end by sequences from the upstream region of the endogenous gene by excluding the actual promoter region of the endogenous gene. The flanking sequences are of sufficient length for successful homologous recombination of the vector DNA with the endogenous gene. Preferably, several kilobases of flanking DNA are included in the homologous recombination vector. Upon homologous recombination between the vector DNA and the endogenous gene in a host cell, the endogenous promoter is replaced by the recombinant promoter. Thus, expression of the endogenous gene is no longer under the control of its endogenous promoter but rather is placed under the control of the transcription unit in accordance with the present invention.

In another embodiment, an operator sequence may be inserted elsewhere within an endogenous gene, preferably within a 5′ or 3′ regulatory region, via homologous recombination to create an endogenous gene whose expression can be regulated by a transcriptional activator or repressor described herein. For example, one or more binding sequences of a chimeric transcription factor of the present invention can be inserted into a promoter or enhancer region of an endogenous gene such that promoter or enhancer function is maintained.

When muscle is the target tissue for gene therapy, direct intramuscular injection of either viral- or non-viral vectors is one of the preferred modes for transgene delivery in vivo. In particular, direct intramuscular injection of viral or non-viral vectors encoding: i) antigens from viruses, bacteria or protozoans result in the protection against a subsequent challenge with the corresponding pathogen; ii) tumor-specific antigens result in protection of mice against challenges with tumorigenic cells expressing the corresponding antigen; iii) secreted proteins result in delivery into the bloodstream (Marshall, D. J. and Leiden, J. M., 1998, Curr. Opin. Genet. Dev., 8, 360-365).

A still further aspect provides a method of introducing the nucleic acid into a host cell in vitro. The in vitro introduction can be used for ex vivo gene therapy and the non-gene therapy methods discussed above. The introduction, which may (particularly for in vitro introduction) be generally referred to without limitation as “transformation”, may employ any available technique. For eukaryotic cells, suitable techniques may include calcium phosphate transfection, DEAE-Dextran, electroporation, liposome-mediated transfection and transduction using viruses as disclosed above. In addition, viruses such as vaccinia, and baculovirus (for insect cells), can also be used. For bacterial cells, suitable techniques may include calcium chloride transformation, electroporation and transfection using bacteriophage. As an alternative, direct injection of the nucleic acid could be employed. Marker genes such as antibiotic resistance or sensitivity genes may be used in identifying clones containing nucleic acid of interest, as is well known in the art.

4. Suitable Composition

Suitable compositions may be needed for the delivery of the polynucleotides encoding the gene switch to the target cells or tissues of the mammals to be treated, as well as the administration of the exogenous ligand to the mammals.

A composition according to the present invention that is to be given to an individual, administration is preferably in a “prophylactically effective amount” or a “therapeutically effective amount” as the case may be, although prophylaxis may be considered therapy), this being sufficient to show benefit to the individual. The actual amount administered, and rate and time-course of administration, will depend on the nature and severity of what is being treated. Prescription of treatment, e.g. decisions on dosage etc, is within the responsibility of general practitioners and other medical doctors. A composition may be administered alone or in combination with other treatments, either simultaneously or sequentially dependent upon the condition to be treated.

Pharmaceutical compositions according to the present invention, and for use in accordance with the present invention, may include, in addition to active ingredient, a pharmaceutically acceptable excipient, carrier, buffer, stabiliser or other materials well known to those skilled in the art. Such materials should be non-toxic and should not interfere with the efficacy of the active ingredient. The precise nature of the carrier or other material will depend on the route of administration, which may be oral, or by injection, e.g. cutaneous, subcutaneous or intravenous.

Pharmaceutical compositions for oral administration may be in tablet, capsule, powder or liquid form. A tablet may include a solid carrier such as gelatin or an adjuvant. Liquid pharmaceutical compositions generally include a liquid carrier such as water, petroleum, animal or vegetable oils, mineral oil or synthetic oil. Physiological saline solution, dextrose or other saccharide solution or glycols such as ethylene glycol, propylene glycol or polyethylene glycol may be included.

For intravenous, cutaneous or subcutaneous injection, or injection at the site of affliction, the active ingredient will be in the form of a parenterally acceptable aqueous solution which is pyrogen-free and has suitable pH, isotonicity and stability. Those of relevant skill in the art are well able to prepare suitable solutions using, for example, isotonic vehicles such as Sodium Chloride Injection, Ringer's Injection, Lactated Ringer's Injection. Preservatives, stabilisers, buffers, antioxidants and/or other additives may be included, as required. Liposomes, particularly cationic liposomes, may be used in carrier formulations.

Examples of techniques and protocols mentioned above can be found in Remington's Pharmaceutical Sciences, 16th edition, Osol, A. (ed), 1980.

The composition may be administered in a localized manner to a tumor site or other desired site or may be delivered in a manner in which it targets tumor or other cells.

Targeting therapies may be used to deliver the composition more specifically to certain types of cell, by the use of targeting systems such as antibody or cell specific ligands. Targeting may be desirable for a variety of reasons, for example if the agent is unacceptably toxic, or if it would otherwise require too high a dosage, or if it would not otherwise be able to enter the target cells.

A composition may be administered alone or in combination with other treatments, either simultaneously or sequentially dependent upon the condition to be treated, such as cancer, virus infection or any other condition in which an effect mediated by activity of the fusion protein is desirable.

5. Host Cells

Thus, a further aspect of the present invention provides a host cell containing heterologous nucleic acid as disclosed herein.

The host cell can be, for example, a mammalian cell (e.g., a human cell), a yeast cell, a fungal cell or an insect cell. Moreover, the host cell can be a fertilized non-human oocyte, in which case the host cell can be used to create a transgenic organism having cells that express the transcriptional inhibitor fusion protein.

According to an embodiment of the present invention, the mammalian host cell is a cell in a mammal, human or non-human. The nucleic acid is introduced to the cell via approaches of in vivo or ex vivo gene delivery.

Hence, the invention is applicable to normal mammalian cells, such as cells to be modified for gene therapy purposes or embryonic cells modified to create a transgenic or homologous recombinant animal. Examples of cell types of particular interest for gene therapy purposes include hematopoietic stem cells, myoblasts, hepatocytes, lymphocytes, muscle cells, neuronal cells and skin epithelium and airway epithelium. Additionally, for transgenic or homologous recombinant animals, embryonic stem cells and fertilized oocytes can be modified to contain nucleic acid encoding a transactivator or repressor fusion protein.

In addition to normal cells, the invention is applicable to cell lines. According to an alternative embodiment of the present invention, the mammalian host cell is a cultured cell, or a cell from a cell line. Examples of mammalian cell lines which may be used include CHO 30 dhfr-cells (Urlaub and Chasin (1980) Proc. Natl. Acad. Sci. USA 77:4216-4220), 293 cells (Graham et al. (1977) J. Gen. Virol. 36: pp 59) and myeloma cells like SP2 or NSO (Galfre and Milstein (1981) Meth. Enzymol. 73(B):3-46).

Preferably, the target cells or tissues do not contain endogenous active transcription factors that bind the binding site. For instance, the fact that HNF-1 is not expressed in muscles is of relevance for gene therapy purposes in accordance with the present invention. According to a preferred embodiment, the target cells are muscle cells, and the DNA binding domain of the chimeric transcription factor is from HNF-1. Thus, the gene of interest would not be stimulated by endogenous transcription factor.

Nucleic acid encoding a chimeric transcription factor according to the present invention can also be transferred into a fertilized oocyte of a non-human animal to create a transgenic animal which expresses the transcription factor in one or more cell types.

Aspects of the invention further provide non-human transgenic organisms, including animals, that contain cells which express chimeric transcriptional factor of the invention (i.e., a nucleic acid encoding the transactivator or repressor is incorporated into one or more chromosomes in cells of the transgenic organism).

The method for producing such transgenic cells is not particularly germane to the present invention and any method suitable for the target cell may be used; such methods are known in the art, including cell specific transformation.

### EXAMPLES

Examples are provided below to further illustrate different features of the present invention. The examples also illustrate useful methodology for practicing the invention. These examples do not limit the claimed invention.

Materials and Methods

Plasmid Constructs

The yeast expression vector pGBT9-GAL4 DBD/ERα LBD/VP16AD was obtained by inserting a DNA cassette coding for the chimeric transcription factor into pET23b vector (Novagen) and then transferring the cassette into the pGBT9 yeast expression vector (Clontech).

A HindIII-HincII DNA fragment containing the coding sequence for the minimal GALA DNA-binding domain (DBD) (aa 1-93) was excised from the plasmid pAS2-1 (Clontech) and inserted into pET23b vector digested with the same enzymes, thus generating pET-GAL4DBD.

A SacI-BamHI DNA fragment containing the coding sequence for the minimal VP16 activation domain (AD) (aa 424-490) was excised from the plasmid pUHD172-1neo and inserted into pET-GAL4DBD digested with the same enzymes, thus generating pET-GALA DBD/VP16AD. In this plasmid the coding sequence for VP16 AD was in frame downstream of the GAL4 DBD coding sequence and a two-amino acid junction (TE) was introduced during cloning.

The hERα LBD coding sequence (aa 282-595) was obtained by PCR amplification using as template the plasmid phERα/BSKS(−) containing the full-length hERα ORF (1-595) and the following DNA primers: forward, 5′-GGAATTCGTTGACCGGGTCTGCTGGAGACATG-3′ (SEQ ID NO: 45); reverse, 5′-GGAATTCGAGCTCTGAACCAGACCCGACTGTGGCA GGGAAACC-3′ (SEQ ID NO: 46). The obtained DNA fragment was digested with HincII and SacI and inserted into pET-GAL4DBD/VP16AD digested with the same enzymes, thus generating pET-GAL4DBD/hERα LBD/VP16AD. In this construct, the hERα LBD coding sequence was cloned in frame with the C-terminus of GALA DBD coding sequence through a two-amino acid linker (TG) and with the N-terminus of VP16 AD to which it was joined by a GSGSE linker.

The construct pGBT9-GAL4DBD/ERα LBD/VP16AD was obtained by excising the DNA cassette from pET-GAL4DBD/hERα LBD/VP16AD using XhoI and BamHI and cloning into the pGBT9 vector digested with the same enzymes.

pGBT9-GAL4 DBD/ERα LBD G(521)R/VP16AD was obtained by substituting the wt NcoI-BspMI-digested DNA fragment contained in pET-GAL4DBD/hERα LBD/VP16AD with the same fragment carrying the mutated codon obtained from pGEX-ERα LBD G(521)R (see below) digested with the same enzymes. GAL4 DBD/ERα LBD G(521)R/VP16AD DNA-cassette was then transferred to the pGB T9 vector by XhoI and BamHI digestion, as described above.

pGBT9-GAL4DBD/ERα LBD L(384)M/VP16AD was obtained by PCR-mediated site-directed mutagenesis using the wt construct as template and the following DNA primers: forward (EagI,s), 5′-GTCCCTGACGGCCGACCAGATGG TCAGTGCCTTGTTGGATGCTGAGCCC-3′ (SEQ ID NO: 47); reverse [L(384)M Nco,as], 5′-GTGCTCCATGGAGCGCCAGACGAGACCAATCATCAGGATCTC CATCCAGGC-3′ (SEQ ID NO: 48). The amplified mutated DNA fragment was digested with EagI and NcoI and inserted into pGBT9-GAL4 DBD/ERα LBD/VP16AD digested with the same enzymes.

To introduce the G(521)R mutation in both pGBT9-GAL4 DBD/ERα LBD L(384)M/VP16AD and pGBT9 GAL4 DBD/ERα LBD L(384)M, M(421)G/VP16AD a StuI-SacI fragment containing the mutation was excised from pGBT9-GAL4 DBD/ERα LBD G(521)R/VP16AD and inserted into the corresponding recipient vectors digested with the same enzymes.

To clone the alternative mutations at the G-521 and H-524 positions of the selected mutant pGBT9-GAL4DBD/ERα LBD L(384)M, M(421)G/VP16AD PCR-mediated site-directed mutagenesis was performed using the wt construct as a template and the StuI,s oligonucleotide as forward primer (5′-CAAGGCAGGCCTGACCCTGCAGCAGCAGCACC-3′) (SEQ ID NO: 49) in combination with each of the following mutagenic reverse primers: GV,BspMI,as, 5′-GCATCTCCAGCAGCAGGTCATAGAGGGGCACCACGTTCTTG CACTrCATGCTCGTACAGATGCTCCATCACTTTG-3′ (SEQ ID NO: 50); GL,BspMI,as and GM, BspMI,as had the same sequence with the exception of the mutagenized triplet that was CAG and CAT, respectively; HV,BspMI,as, 5′-GCATCTCCAGCAGCAGGTCATAGAGGGGCACCACGTTCTTGCACTTCAT GCTGTACAGCACCTCCATGCCTTT-3′ (SEQ ID NO: 51). Each of the mutagenized fragments was digested with StuI and BspMI and cloned into pET-GAL4 DBD/hERα LBD/VP16AD cut with the same enzymes.

Each amino acid substitution was cloned into pGBT9-GAL4 DBD/ERα LBD L(384)M, M(421)G/VP16AD by digesting each mutated pET-construct with StuI and BsmI and transferring the corresponding mutated fragments in the recipient vector digested with the same enzymes.

To introduce the D(351)A mutation into pGBT9-GAL4DBD/ERα LBD L(384)M, M(421)G, H(524)V/VP16AD the suitable mutated DNA fragment was obtained by PCR-mediated mutagenesis using as template pGBT9-GAL4DBD/ERα LBD L(384)M/VP16AD plasmid and the forward primer (DA,Hind,s), 5′-CTTCAGTGAAGCTTCGATGATGGGCTTACTGACCAACCTGGCAGCCAGGG-3′ (SEQ ID NO: 52) coupled with the reverse primer L(384)M Nco,as (see above). The resulting fragment was digested with HindIII and NcoI and inserted into pGEX-ERα LBD L(384)M (see below) cut with the same enzymes. This latter construct was then digested with EagI and NcoI and the restriction fragment was cloned into pGBT9-GAL4 DBD/ERα LBD L(384)M, M(421)G, H(524)V/VP16AD digested with the same enzymes.

Both plasmids pGEM-hERα LBD and pGEX-ERα LBD contain the DNA sequence coding for hERα LBD (aa 303-595) that in pGEX is in frame with the GST coding sequence. The construct pGEM(RI)-hERα LBD was obtained by inserting an EcoRI site in the polylinker of pGEM-hERα LBD between NotI and SalI sites downstream of the hERα LBD coding sequence. The PCR reaction was performed using pGEM-hERα LBD as template and the following primers: forward, 5′-CTATGACCTGCTGCTGGAGATGCTGGACG-3′ (SEQ ID NO: 53); reverse, 5′-CATATGGTCGAC GAATTCGCGGCCGCAC-3′ (SEQ ID NO: 54). The DNA fragment was digested with BspMI and SalI and inserted in pGEM-hERα LBD opened with the same enzymes.

The construct pGEM(R¹)-hERα LBD G(521)R was obtained by PCR-mediated site-directed mutagenesis using pGEM(R¹)-hERα LBD as template, StuI,s as forward primer (see above) and GR,BspMI,as (5′-GCATCTCCAGCAGCAGGT CATAGAGGGGCACCACGTTTCTGCACTrCATGCTGTACAGATGCTCCATGCGTTG-3′) (SEQ ID NO: 55) as reverse primer. The mutated DNA fragment was digested with StuI and BspMI and inserted into pGEM(RI)-hERα LBD digested with the same enzymes.

The construct pGEX-ERα LBD G(521)R was obtained by transferring the NcoI-EcoRI DNA fragment from pGEM-hERα LBD G(521)R to pGEX-ERα LBD digested with the same enzymes.

The construct pGEX-ERα LBD L(384)M was obtained by digesting the plasmid pGBT9-GAL4 DBD/ERα LBD L(384)M/VP16AD with EagI and NcoI and inserting the restriction fragment containing the mutated codon into pGEX-ERα LBD digested with the same enzymes.

The construct pGEX-ERα LBD L(384)M, M(421)I was obtained by PCR-mediated site specific mutagenesis using pGEX-ERα LBD L(384)M as template, the forward primer EagI,s (see above) and the following reverse primer. 5′-GTCC AAGATCTCCACGATGCCCTCTACAC-3′ (SEQ ID NO: 56). The DNA fragment was digested with EagI and BglII and inserted in pGEX-ERα LBD L(384)M cut with the same enzymes.

Selected combinations of mutations were transferred from the corresponding pGBT9-GAL4 DBD/ERα LBD L(384)M /VP16AD library vectors to the pGEX-ERα LBD plasmid by excision of a DNA fragment coding for both the L(384)M substitution and the desired selected mutations using HindIII and StuI and replacing the corresponding wt fragment in pGEX-ERα LBD digested with the same enzymes.

Mammalian expression vectors coding for wt and mutated versions of GAL4DBD/ERα LBD L(384)M/VP16AD were obtained by digesting with XhoI and BamHI the corresponding pGBT9-constructs and cloning the restriction fragment in pM vector (Clontech) digested with the same enzymes.

The 5GAL4UAS-pSEAP-reporter gene was constructed by digesting the plasmid pG5CAT (Clontech) with BamHI and EcoRI followed by filling-in with the Klenow enzyme. The restriction fragment containing five GAL4 UAS repeats and the E1b minimal promoter was then cloned upstream of the SEAP coding region into the pSEAP2-Basic plasmid (Clontech) digested with HindIII followed by Klenow filling-in.

Plasmid pV1j/HEAm45.2 was obtained after several cloning steps. We first generated plasmids pHEAwt and pBS/ERwtLBD. To obtain pHEAwt, a fragment scanning aa 303-595 of the LBD of hERα was obtained by PCR amplification with primers 5′-GATATC CAAGAACAGCCTGGCCTTGTCCCTGACG-3′ (SEQ ID NO: 57) and 5′-ACTAGTGAATTCGACTGTGGCAGGGAAACCCTCTGCCTCCC-3′ (SEQ ID NO: 58), using plasmid phERα/BSKS(−) as a template. Digestion of the amplified fragment with enzymes XbaI and EcoRI released a fragment spanning aa 379-595 of the wt LBD. This was used as a substitute for the corresponding region of plasmid pHEA-1, which has been previously described (Roscilli et al., 2002 Mol Ther. 5:653-663), thus obtaining plasmid pHEAwt.

From plasmid pHEAwt, the 303-595 region of the hERα wt LBD was excised as an EcoRV-EcoRI fragment and introduced into the plasmid pBlueScript/HindIII-, in which the HindIII site had been removed by digestion with the enzyme HindIII, filling-in by Klenow and re-ligation. The plasmid obtained was called pBS/ERwtLBD.

Plasmid pBS/ERM45 was then constructed by inserting a HindIII-StuI fragment from plasmid pGBT9-GAL4DBD/ERαLBD L(384)M, M(421)G/VP16AD into plasmid pBS/ERwtLBD digested with the same enzymes. Plasmid pBS/ERM45 therefore contains hERα LBD (from aa 303 to aa 595) carrying the L(384)M, M(421)G mutations. This mutated LBD was then excised from pBS/Erm45 as an EcoRV-EcoRI fragment and used as a substitute for the wt LBD of plasmid pHEAwt, thus obtaining plasmid pV1j/HEAm45.

Finally, a fragment spanning aa 1-406 of HLAm45 was obtained by digesting plasmid pV1j/HEAm45 with BglII and introduced into plasmid pHEA1 (Roscilli et al., 2002 Mol Ther. 5:653-663) digested with the same enzyme. The plasmid obtained was called pV1j/HEAm45.2.

All DNA constructs were verified by automatic sequencing using suitable oligonucleotides.

Expression in Bacteria and Purification of GST-ER LBD Fusion Proteins

E. coli BL21 cells (CODONPLUS™ DE 3-RIL, Stratagene) were transformed with suitable pGEX-ERα LBD expression plasmids. A 2-liter liquid culture derived from a single transformed bacterial colony was grown at 37° C. to A₆₀₀=0.8 in M9 modified minimal medium (5 g/L glucose, 1 g/L ammonium sulphate, 100 mM potassium phosphate pH 7, 5 μM biotin, 7 μM thiamine, 0.5% casamino acids, 0.5 mM MgSO₄, 0.5 mM CaCl₂, 13 μM FeSO₄, 50 mg/L ampicillin). It was then cooled to 18° C. and induced with 600 μM IPTG (isopropyl-b-thiogalactopyranoside) for 22 hours at 18° C. All subsequent operations were performed at 4° C. unless otherwise indicated.

Cells were harvested and disrupted with a Microfluidizer (Model 110-S) in 200 ml of a buffer containing 50 mM Tris-HCl pH 7.5, 0.5 mM EDTA, 10% glycerol, 0.2 M NaCl, 0.4% n-Dodecyl β-D-maltoside (Calbiochem), 5 mM DTT, 1 mM PMSF, and COMPLETE (Boehringer) protease inhibitor mixture. Insoluble material was pelleted at 27,000×g for 30 min in a Sorvall SS34 rotor. The clarified supernatant containing between 30% and 50% of the recombinant protein was either stored in aliquots at −80° C. after shock freezing in liquid nitrogen to be used directly in in vitro binding assays or further purified.

The supernatant was loaded on two connected 5 ml-High Trap GST-Sepharose columns (Pharmacia) pre-equilibrated in lysis buffer containing 50 mM Tris-HCl pH 8 and 0.1% n-Dodecyl β-D-maltoside. The GST-fusion protein was eluted in the same buffer supplemented with 10 mM reduced glutathione and further purified on a Superdex 200 26/60 gel filtration column (Pharmacia) equilibrated with lysis buffer containing 0.1% n-Dodecyl β-D-maltoside. The peak fraction corresponding to the purified GST-ER LBD homodimeric form at a concentration of approximately 2 μM was stored in suitable aliquots at −80° C. after shock-freezing in liquid nitrogen.

In Vitro Ligand-Binding Assays

Determination of ligand affinity for the GST-ER LBD polypeptides was done by a competitive radiometric binding assay using tritium-labeled estradiol (³[M]E₂) (Amersham, 158 Ci/mmol, 558 mCi/mg) as tracer.

Microplates (Basic Flashplates, NEN) wells were coated for 12 hrs at 4° C. with 100 μl of PBS containing anti-GST antibodies (Amersham) at a concentration of 5 μg/ml. After washing three times with 200 μl of PBS, the background was reduced by saturating with 200 μl of PBS containing 1% BSA for 3 hrs at 4° C. Wells were then washed three times with 200 μl of lysis buffer containing 0.1 M NaCl and 0.2% n-Dodecyl BD-maltoside (assay buffer).

Suitable amounts of crude E. coli supernatants containing the GST-ER LBD proteins (2-10 μl) or of purified polypeptides (8 nM) were bound to the anti-GST antibody-coated wells for 1 hr at 23° C. in 200 μl of the assay buffer with constant agitation. After three more washes with 200 μl of the same buffer, the ligand-binding reaction was set up in 195 μl of assay buffer containing 2 nM-10 nM 3[H]E₂ and 5 μl of DMSO or of suitable dilutions of the test compounds in DMSO. Incubation was for 2 hrs at 23° C. with constant shaking, followed by SPA radioactivity measurement using a microplate scintillation and luminescence counter (Top Count NXT, Packard). IC₅₀ values were obtained by multiparameter logistic fitting of the experimental data with the aid of a Kaleidagraph software.

Yeast Strains and Growth Conditions

Yeast strain CG-1945 (Clontech) was used as a reporter host strain for the display and screening of the library. It contains both the HIS3 and lacZ reporter genes under the control of a GALA-responsive UAS integrated into the genome. Yeast strain Y187 (Clontech) was instead used for quantitative transcriptional β-galactosidase assays. It contains only the lacZ reporter gene (probably present in two copies), which is expressed at higher levels than in CG-1945 for being under the control of the natural intact GAL1 promoter instead of the synthetic UAS_{G 17-mer (x3)} consensus sequence. Both strains are gal4⁻ and gal80⁻ and were propagated at 30° C. in YPD medium.

Yeast strains transformed with pGBT9 plasmids were grown and stored in SD minimal medium (Clontech) supplemented with −Trp amino acid mixture. Growth selection for the nutritional reporter HIS3 was performed on agar plates in SD minimal medium supplemented with −His/−Trp amino acid mixture and either 15-30 mM (single plasmid transformants) or 70 mM (library clones pool) of 3-amino-1,2,4-triazole (3-AT), a competitive inhibitor of the HIS3 protein.

In vivo β-galactosidase assays were performed on agar plates in SD minimal medium supplemented with −Trp amino acid mixture, X-gal (80 mg/L) and 1×BU salts (10x=70 g of Na₂PO₄⁻H₂O and 30 g of NaH₂PO₄, pH 7). Growth selective and X-gal plates were supplemented with estradiol, tamoxifen (Sigma), tetrahydrofluorenone compounds or DMSO at the indicated concentrations.

Transformation of Yeast Cells

Yeast cells were transformed using the Li Ac procedure and the YEASTMAKER Yeast transformation kit (Clontech) following the manufacturer protocol.

For single plasmid small-scale transformations 0.1 μg of DNA were used to transform 0.1 ml of yeast competent cells resulting in transformation efficiencies of approximately 10⁵ colony-forming units (cfa) per μg of DNA.

To set up transformation conditions for homologous recombination of the pGBT9-GAL4 DBD/ERα LBD L(384)M/VP16AD with mutagenized PCR products, the expression vector was linearized by restriction digestion with BglII and half of it was treated with Klenow polymerase prior of gel purification. 100 ng of each of the two versions of the digested plasmid were then transformed as outlined with 200 ng, 300 ng or 400 ng of a 160 bp PCR fragment obtained using wild-type oligonucleotides corresponding to the degenerated oligonucleotides designed to construct the mutated library.

Co-transformation of 100 ng of linearised blunt-ended vector with 300 ng of PCR fragment showed an efficiency of approximately 10⁵ per μg of DNA that was 100-fold higher than background with the recipient vector alone. This condition was used for the library-scale transformation procedure in which 60 μg of the mutagenized fragment collection and 20 μg of linearised recipient vector were used to transform 1 ml of yeast cells with an estimated efficiency of 5×10⁴ cfu per μg of DNA.

Library Construction and Selection

Degenerate oligonucleotide mixtures were synthesised by a split-pool strategy. At each of the positions corresponding to Leu391, Phe404, Met421, Ile424 or Leu428, the previously synthesized column material was split into 10 individual pools, the 10 possible codons were synthesized separately and the pools mixed again together. Codons used were: GGC(Gly), GCC(Ala), TGC(Cys), GTG(Val), ATC(Ele), CrG(Leu), ATG(Met), TTC(Phe), TAC(Tyr), TGG(Trp).

A slightly higher quantity of pooled column material (15% of total) was used to synthesize the wt codon thus leaving 9.4% for each of the other 9 remaining codons. This “wt-spiking” increased the relative frequency of clones with low number of substitutions (1 or 2) with respect to clones having 4 or 5 substitutions that represent the majority of the library clones. Thus, the likelihood that single- or double-mutation clones would be lost during the synthesis, construction or selection of the library should be significantly decreased. While in a homogeneous “10%-library” the 45 possible combinations of single-substitution clones would have represented only 0.045% of the total library, the use of 15% wt-codon increases this fraction to 0.2152%, i.e. the clones were about 5-fold as frequent represented in the library. Consequently, the fraction of clones with 5 substitutions (59049 possible combinations) dropped from about 59% to about 44%.

Preparative amounts of mutated LBD fragments were synthesised by PCR amplification of 6 μg of pGBT9-GAL4DBD/hERa LBD/VP16AD DNA template by including 2.1 nmol of each of the degenerated oligonucleotide mixes in a total reaction volume of 6 ml containing 250 mM dNTPs, 5% DMSO, 600 μl of Pfu 10× buffer and 300 units of Pfu polymerase (Stratagene). The PCR amplification consisted of 25 cycles at 95° C. for 1 min, 65° C. for 1 min and 72° C. for 2 min.

60 μg of the mutagenized 160 bp product mixture were purified on QIAquick spin columns (Qiagen) and used in a scaled-up co-transformation experiment as outlined together with 20 μg of linearised recipient vector. 30 ml of the co-transformation reaction were spread on twenty 23-cm×23-cm −Trp selective plates, colonies were harvested after 3 days of growth at 30° C. and 1 ml-glycerol stocks of the amplified library were made. Suitable dilutions of the co-transformation mixture were spread on 100-mm plates to control the efficiency of homologous recombination and to determine the library titer (5×10⁴ cfu per μg of DNA). Glycerol stocks were also titered and resulted to contain 1.2×10⁵ cfu per μl. 1×10⁵ cfu were spread on each of twenty 100-mm −His/−Trp selective plates containing 70 mM 3-AT and either 1 μM (first experiment) or 10 μM (second experiment) of CMP1 (a total of 2×10⁶ cfu per experiment were plated).

After 6 days of growth at 30° C. the average number of His⁺ colonies per plate was 200 in the first experiment and 60 in the second experiment. 80 colonies out of 200 of the first screening and all colonies of the second screening were streaked out from each plate on X-gal 100 mm plates containing either DMSO or CMP1 at the concentration of 1 μM or 10 μM, respectively (a replica on −Trp master plates was also performed). After 3 days of growth at 30° C. all clones that resulted blue in the presence of the compound and white on control plates were streaked out of corresponding master plates on X-gal plates containing either DMSO or CMP1 at a 10-fold lower concentration and on −Trp plates. This procedure was reiterated down to a compound concentration of 0.1 μM at which 28 and 31 independent positive clones, respectively, were collected for further analysis.

Plasmid Rescue from Yeast

Colonies corresponding to the mutants of interest were grown to saturation at 30° C. for 16 hrs in 2 ml of −Trp SD minimal medium. Cells from a 1.3 ml culture fraction were collected by centrifugation and resuspended by vortexing in 0.2 ml of protoplasting buffer (100 mM Tris-HCl pH 7.5, 10 mM EDTA, 14.4 mM β-mercaptoethanol) containing 400 μl of a 40 units/μl Lyticase (Sigma) solution. After cell walls were dissolved by incubation for 2 hr at 37° C., 200 μl of lysis solution (0.2 M NaOH, 1% SDS) were added and samples were incubated at 65° C. for 20 min, then put rapidly on ice.

Samples were mixed with 200 μl of 3 M K-acetate pH 5.4, incubated on ice for 15 min, and spun for 3 min at 13,000 rpm in an Eppendorf microcentrifuge. Plasmid DNA was recovered from supernatants by precipitation with 0.6 volumes of isopropanol. Rescued plasmids were both transformed in electro-competent E. coli DH12S cells and directly sequenced by PCR-amplification of a 360 bp fragment using oligonucleotide primers (5′-CTGACCAACCTGGCAGACAG-3′ (SEQ ID NO: 59); 5′-GGACTCGGTGGATATGGTCC-3′ (SEQ ID NO: 60)) annealing 100 bp upstream and downstream of the mutagenized insert, respectively. The amplified fragments were purified on QIAquick spin columns and subjected to automatic sequencing using either of two sequencing primers (5′-GTTCACATGATCAACTGG GCG-3′ (SEQ ID NO: 61); 5′-GAGACTTCAGGGT GCTGGAC-3′ (SEQ ID NO: 62) that annealed 70 bp from the mutagenized insert boundaries.

Yeast Protein Extracts and Western Blot Analysis

The Urea/SDS method was followed to prepare yeast protein extracts suitable to evaluate mutant protein expression by Western blot analysis using anti VP16 AD polyclonal antibodies (Santa Cruz).

Quantitative β-Galactosidase Assays

The assays were performed according to Clontech yeast protocol handbook. Single colonies were grown to saturation for 16 hrs at 30° C. in −Trp SD minimal medium, cells were collected by centrifugation and then diluted in YPD medium to an optical density of 0.04 at 600 nM. Subcultures of 5 ml-volume were set up and allow to grow for 7 hrs at 30° C. in the presence of DMSO or various ligand concentrations until they reached mid-log phase (OD₆₀₀=0.4-0.5).

Cells from 1.5 ml of culture (two duplicates for each sample) were pelleted, washed in lacZ buffer (10 mM KCl, 1 mM MgSO₄, and 100 mM phosphate pH 7), and resuspended in 300 μl of lacZ buffer. 25-100 μl of the suspension were lysed by three freeze/thaw cycles, mixed with 0.7 ml of lacZ buffer containing 50 mM β-mercaptoethanol and the enzymatic reaction was started by the addition of 160 μl of a 4 mg/ml ONPG (Sigma) solution in lacZ buffer.

The reaction was performed at 30° C. until the yellow color developed and was stopped by the addition of 0.4 ml of 1 M Na₂CO₃, centrifuged and quantified by reading the optical density at 420 nm. Units of β-galactosidase were then defined as (1000×OD₄₂₀) divided by [assay duration in min×(0.1 ml×concentration factor)×OD₆₀₀]. Dose-response data were analyzed using a non-linear regression analysis with the aid of a Kaleidagraph software.

Cell Culture, Transfections Mid SEAP Assays

All cell culture experiments were performed using phenol red-free Dulbecco's modified Eagle' medium (DM, Gibco BRL) supplemented with 10% dextran charcoal-treated (Sigma) fetal bovine serum (FBS, GibcoBRL). Twenty-four hours prior transfection HeLa cells were seeded at a density of 200,000 cells per well in 6-well plates. Transfections were performed using FuGENE 6 Transfection reagent (Roche) according to the manufacturer's instructions.

The cells were transfected with 1 μg of the reporter plasmid 5GAL4UAS-pSEAP, 0.1 μg pCMV-Luc as internal control, variable amounts of pGBT9-GAL4DBD/ER LBD/VP16AD expression vectors and variable amounts of carrier pSEAP2-Basic DNA to normalize all samples to 2 μg of total plasmid DNA. DNA was mixed to 6 μl of FuGENE Reagent diluted in 100 μl of serum-free medium and added to cells. Seven hours later cells were provided with fresh medium and 24 hours after transfection the different ligands (or DMSO vehicle) were added at the indicated concentrations in fresh medium.

Cells were then grown for 24 hours in the presence of the inducers and submitted to protein extraction for luciferase assays while supernatants were collected and processed for the detection of secreted alkaline phosphatase (SEAP). SEAP activity was evaluated using a commercially available assay (Tropix Phospha-Light system) following the manufacturer's guidelines.

Reactions were performed in microplates and SEAP activity was measured as light emission using a microplate scintillation and luminescence counter (Top Count NXT, Packard). Values were subtracted of the background value obtained by measuring endogenous alkaline phosphatase in un-transfected cell medial SEAP values were normalized for differences in the transfection efficiency, which was determined on the basis of luciferase activity. Then they were converted to SEAP concentration values (ng/ml) by comparison with standard activity curves obtained with purified human placental alkaline phosphatase (Sigma).

To evaluate the effect of compounds on the m45.2 chimera transcriptional activity, HeLa cells were seeded 18 hrs before transfection in Swell plate (3×10⁵ cells/well), and then transfected with 1 μg of plasmid DNA per well (0.5 μg transactivator+0.5 μg reporter) by using Lipofectamine (Gibco), according to manufacturer's instructions. 100 ng of the luciferase reporter plasmid were included in the transfection mixture as an internal control for transfection efficiency. At six hours after transfection, the culture medium was changed and cells were treated or not with the various ligands. After additional 24 hours, the medium was harvested and analyzed for the expression of human SEAP, as described above. SEAP levels were normalized against the luciferase activity measured in cell extracts. Dose-response data were analyzed as outlined above.

### Example 1

### Synthesis of 9a-Benzyl-7-Hydroxy-4-Methyl-1,2,9,9a-Tetrahydro-3h-Fluoren-3-One (CMP4)

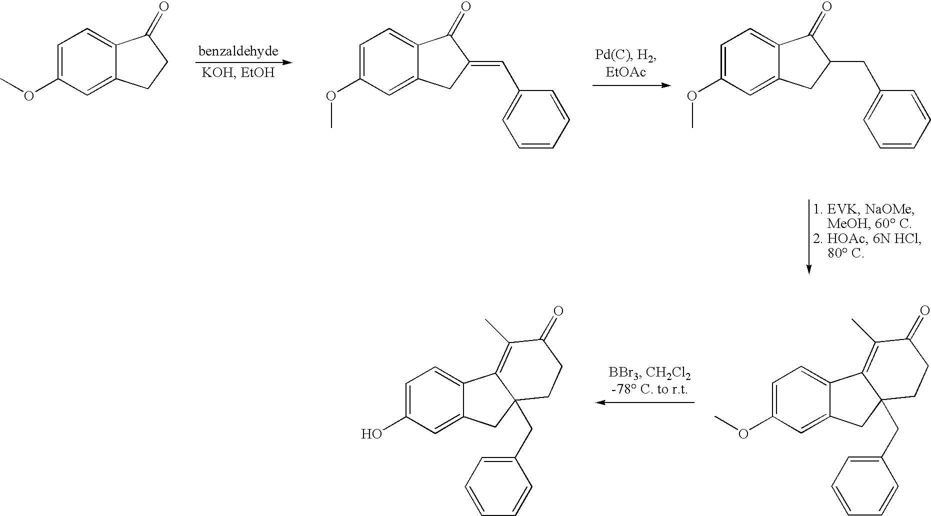

### Step 1: 5-methoxy-2-benzylidene-1-indanone

To a stirred suspension of 5-methoxyindanone (1 g, 5.89 mmol) and benzaldehyde (0.79 g, 7.4 mmol) in EtOH (6 mL) at room temperature was added KOH (0.6 g). After 20 h the formed precipitate was filtered off, washed with EtOH and dried under high vaccum to afford the title compound (1.41 g).

¹H NMR (DMSO-d₆, 300 MHz) δ 3.90 (s, 3H), 4.10 (s, 2H), 7.04 (m, 1H), 7.12 (m, 1H), 7.42-7.58(m, 4H), 7.72-7.83 (m, 2H).

### Step 2: 5-methoxy-2-benzyl-1-indanone

A suspension of the product from step 1 (777 mg) and palladium on charcoal (10%, 78 mg) in EtOAc was stirred under a hydrogen atmosphere (0.4 bar) at 40° C. After 3 h the catalyst was filtered off and the solution was concentrated to dryness under reduced pressure. After flash chromatography on silicagel of the crude product 554 mg of the title compound were obtained as a white solid.

¹H NMR (DMSO-d₆, 300 MHz) δ 2.60-2.78 (m, 2H), 2.93-3.18 (m, 3H), 3.82 (s, 3H), 6.92-6.98 (m, 1H), 7.02 (m, 1H), 7.15-7.30 (m, 5H), 7.58 (d, J=2.5 Hz, 1H).

### Step 3: 7-methoxy-9a-benzyl-1,2,9,9a-tetrahydro-3H-fluoren-3-one

To a solution of 5-methoxy-2-benzyl-1-indanone (0.7 g, 2.78 mmol) and ethyl vinyl ketone (293 mg) in anhydrous methanol (5.5 mL) were added 0.97 mL of a 0.5 M solution of NaOMe in MeOH. The mixture was stirred and heated to 60° C. for 1 h. It was then concentrated to dryness under vacuum and the residue was treated with a 1:1-mixture of HOAc/6 N HCl (30 mL) at 80° C. for 3 h. After cooling to room temperature the mixture was diluted with EtOAc and washed with sat. NaHCO₃. The organic phase was dried over Na₂SO₄, filtered and concentrated to dryness under vacuum to afford a yellow oil, which was chromatographied by flash chromatography (silica gel, eluent CH₂Cl₂/2% EtOAc). The collected product fractions were concentrated to dryness under vacuum and the residue was triturated with Et2O/petroleum ether to afford 339 mg of the title compound as a white solid.

¹H NMR (CDCl₃, 400 MHz) δ 1.90-2.02 (, 11H), 2.15 (s, 3H), 2.16-2.25 (m, 1H), 2.50-2.62 (m, 3H), 2.77-2.93 (m, 2H), 3.08 (d, J=16.1 Hz, 1H), 3.88 (s, 3H), 6.83-6.90 (m, 2H), 7.05-7.12 (m, 2H), 7.19-7.30 (m, 3H), 7.67 (d, J=9.3 Hz, 11H).

### Step 4: 7-hydroxy-9a-benzyl-1,2,9,9a-tetrahydro-3H-fluoren-3-on

To a solution of 7-methoxy-9a-benzyl-1,2,9,9a-tetrahydro-3H-fluoren-3-one (279 mg, 0.877 mmol) in CH₂CL₂ (10 mL) at −78° C. was added 1M BBr₃ in CH₂Cl₂ (2.6 mL, 2.6 mmol). The cooling bath was removed and the solution was stirred at room temperature for 2 hours. The solution was diluted with EtOAc (20 mL), washed with 1N HCl (20 mL), dried (Na₂SO₄), filtered and concentrated under reduced pressure. After flash chromatography on silicagel of the crude product 190 mg of the title compound were obtained as a pale yellow solid.

¹H NMR (DMSO-d₆, 400 MHz) δ 1.85-2.07 (m, 51H), 2.80-2.90 (m, 1H), 2.45 (m, 2H, in part overlaid with DMSO-signal), 2.75-3.00 (m, 3H), 6.70-6.80 (m, 2H), 7.05-7.12 (m, 2H), 7.15-7.28 (in, 3H), 7.57 (d, J=8.2 Hz, 1H).

### Example 2

### Synthesis of 9a-(4-Chlorobenzyl)-7-Hydroxy-4-{4-[2-(1-Piperidinyl)Ethoxy]Phenyl}-1,2,9,9a-Tetrahydro-3h-Fluoren-3-One (CMP8)

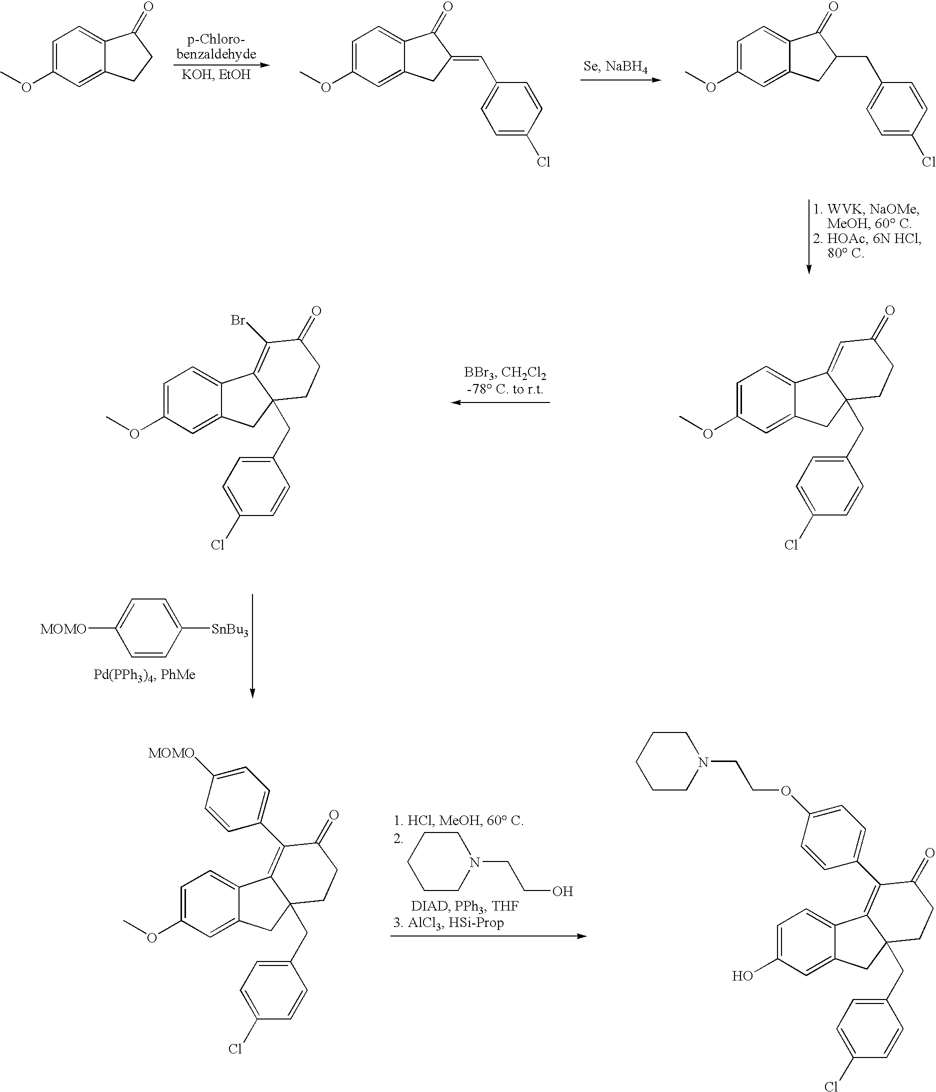

### Step 1: 2-(4-chlorobenzylidene)-5-methoxy-1-indanone

To a stirred suspension of 5-methoxyindanone (1.53 g, 9.01 mmol) and 4-chlorobenzaldehyde (2 g, 14.2 mmol) in EtOH (40 mL) at room temperature was added a 0.5 M solution of sodium methanolate in methanol (5.6 mL). After 3 h the formed precipitate was filtered off, washed with EtOH and dried under high vaccum to afford the title compound 2.37 g of the title compound as a light brown solid.

¹H NMR (DMSO-d₆, 300 MHz) δ 3.88 (s, 3H), 4.07 (s, 2H), 7.03 (m, 1H), 7.17 (m, 1H), 7.44 (m, 1H), 7.55 (m, 2H), 7.70-7.82 (m, 3H).

### Step: 2: 2-(4-chlorobenzyl)-5-methoxy-1-indanone

To a suspension of selenium (180 mg, 2.3 mmol) in anhydrous ethanol (5 mL) was added sodium boronhydride (96 mg, 2.6 mmol) at 0° C. The suspension was stirred for 20 minutes at 0° C. and the formed clear solution was added to a suspension of 2-(4-chlorobenzylidene)-5-methoxy-1-indanone (451 mg, 1.57 mmol) in anhydrous tetrahydrofurane under a nitrogen atmosphere. The mixture was stirred and heated to 50° C. for 2 hours. After cooling to room temperature the mixture was partitioned between 1 M KH₂PO₄ (150 mL) and EtOAc. The organic phase was filtered, dried over Na₂SO₄ and filtered again. After concentration to dryness under reduced pressure 428 mg of the title compound as a brown oil were obtained.

¹H NMR (CDCl₃, 400 MHz) δ 2.65-2.80 (m, 2H), 2.91-3.00 (m, 1H), 3.12 (m, 1H), 3.32 (m, 1H), 3.86 (s, 3H), 6.73 (s, 1H), 7.91 (m, 1H), 7.13-7.30 (m, 4H), 7.71 (d, J=7.9 Hz, 1H). MS: m/z (M+H⁺): 287.0.

### Step 3: 9a-(4-chlorobenzyl)-7-methoxy-1,2,9,9a-tetrahydro-3H-fluoren-3-one

To a solution of 2-(4-chlorobenzyl)-5-methoxy-1-indanone (8.27 mmol) in 50 mL anhydrous tetrahydrofurane under a nitrogen atmosphere were added methyl vinyl ketone (1.345 g, 19.2 mmol) and DBU (0.285 mL, 1.9 mmol). The mixture was stirred at 55° C. for 20 hours. After cooling to room temperature the mixture was partitioned between 1 M KH₂PO₄ and EtOAc. The organic phase was dried over Na₂SO₄ and filtered. After concentration to dryness under reduced pressure 2.66 g of a crude oil were obtained, which was redissolved in 4 mL of anhydrous tetrahydrofurane. Pyrrolidine (0.62 mL, 7.4 mmol) and acetic acid (0.43 mL, 7.5 mmol) were added and the mixture stirred and heated to 55° C. for 4 hours. After cooling to room temperature the mixture was partitioned between 1 M KH₂PO₄ and EtOAc. The organic phase was washed with 1 M Na₂HPO₄, dried over Na₂SO₄ and filtered. After concentration to dryness under reduced pressure 2.11 g of the crude title compound were obtained as an oil and was used without further purification in the next step.

MS: m/z (M+H⁺): 339.0.

### Step 4: 4-bromo-9a-(4-chlorobenzyl)-7-methoxy-1,2,9,9a-tetrahydro-3H-fluoren-3-one

A solution of crude 9a-(4-chlorobenzyl)-7-methoxy-1,2,9,9a-tetrahydro-3H-fluoren-3-one (2.11 g, ca. 6.2 mmol) in CCl₄ (12 mL) was treated with solid NaHCO₃ (2.6 g, 31 mmol). The mixture was cooled in an ice bath and rapidly stirred while bromine (0.322 mL, 6.3 mmol) was added over 6 minutes. After stirring for 1.5 hours at 0° C., the mixture was partitioned between CH₂Cl₂ (300 mL) and water (300 mL). The aqueous phase was extracted with CH₂Cl₂ (100 mL) and the combined organic phases were dried over Na₂SO₄, filtered, and concentrated to dryness under reduced pressure. After flash chromatography on silicagel of the crude product (eluent: petrolether/EtOAc, 5:1) 683 mg of the title compound were obtained as a pale yellow solid.

¹H NMR (CDCl₃, 400 MHz) δ 2.05-2.25 (m, 2H), 2.63-3.10 (m, 6H), 3.89 (s, 3H), 6.78 (s,1H), 6.85-7.22 (m, 5H), 8.45 (d, J=8.6 Hz, 1H).

MS: m/z (M+Er): 416.6/419.0.

### Step 5: 9a-(4-chlorobenzyl)-7-methoxy-4-(4-methoxymethoxy-phenyl)-1,2,9,9a-tetrahydro-3H-fluoren-3-one

A mixture of 4-bromo-9a-(4-chlorobenzyl)-7-methoxy-1,2,9,9a-tetrahydro-3H-fluoren-3-one (683 mg, 1.64 mmol), Pd(PPh₃)₄ (900 mg, 0.777 mmol), and tributyl-(4-methoxymethoxy-phenyl)-stannane (861 mg, 2.02 mmol) in anhydrous toluene (12 mL) was placed under a nitrogen atmosphere and heated with stirring in an oil bath at 100° C. After 4 days, the mixture was cooled to room temperature and evaporated under vacuum to a dark oil (2.208 g). This material was purified by flash chromatography on silica gel, using 4:1 petroleum ether-EtOAc as elutent, to afford 9a-(4-chlorobenzyl)-7-methoxy-4-(4-methoxymethoxy-phenyl)-1,2,9,9a-tetrahydro-3H-fluoren-3-one (140 mg) as a yellow oil.

¹H NMR (CDCl₃, 400 MHz) δ 2.12-2.31 (m, 2H), 2.63-2.80 (m, 3H), 2.82-2.96 (m, 1H), 2.98-3.10 (m, 2H), 3.55 (s, 3H), 3.80 (s, 3H), 5.25 (m, 2H), 6.40 (m, 1H), 6.49 (m, 1H), 6.73 (s, 11H), 7.02-7.70 (m, 8H).

### Step 6: 9a-(4-chlorobenzyl)-4-(4-hydroxyphenyl)-7-methoxy-1,2,9,9a-tetrahydro-3H-fluoren-3-one

A solution of 9a-(4-chlorobenzyl)-7-methoxy-4-(4-methoxymethoxy-phenyl)-1,2,9,9a-tetrahydro-3H-fluoren-3-one (140 mg, 0.295 mmol) in methanol (12 mL) was warmed in an oil bath at 60° C. and treated with aqueous 2N HCl (2.5 mL). The resulting mixture was stirred and heated at 60° C. for two hours, then cooled to room temperature and concentrated under reduced pressure. The residue was partitioned between EtOAc and 1M Na₂HPO₄. The organic phase was dried over Na₂SO₄, filtered and concentrated under vacuum to leave 120 mg of the title compound as a yellow oil.

¹H NMR (CDCl₃, 400 MHz) δ 2.12-2.23 (m, 1H), 2.24-2.33 (m, 1H), 2.62-2.80 (m, 3H), 2.83-3.08 (m, 3H), 3.78 (s, 3H), 6.40-6.51 (m, 2H), 6.72 (s, 1H), 6.80-7.00 (n, 4H), 7.05 (d, J=8.8 Hz, 2H), 7.23 (d, d, J=8.8 Hz, 2).

### Step 7: 9a-(4-chlorobenzyl)-4-[4-(2-piperidine-1-ylethoxy)phenyl]-7-methoxy-1,2,9,9a-tetrahydro-3H-fluoren-3-one

To a solution of 9a-(4-chlorobenzyl)-4-(4-hydroxyphenyl)-7-methoxy-1,2,9,9a-tetrahydro-3H-fluoren-3-one (120 mg, 0.28 mmol), triphenylphosphine (220 mg, 3 eq.) and piperidineethanol (112 μL, 3 eq.) in 1.6 mL of anhydrous tetrahydrofurane at 0° C. was added dropwise a solution of diisopropyl-azodicarboxylate in anhydrous tetrahydrofurane (175 μL, 3 eq., in 600 μL) at 0° C. The solution was stirred for 1 h at 0° C. and for 6 h at room temperature. The product was purified by flash chromatography on silica gel, using 2:1 EtOAc-MeOH as elutent. After removal of the solvents under reduced pressure 96 mg of the title compound were obtained as a yellow oil.

¹H NMR (DMSO-d₄, 400 MHz) δ 1.30-1.55 (m, 6H), 2.14 (n, 2H), 2.45 (n, 4H, in part covered by solvent signal), 2.67 (m, 4H), 2.73 (m, 1H), 2.86 (m, 1H), 3.02 (m, 2H), 3.70 (s, 31), 4.10 (t, J=6.5 Hz, 2H), 6.21 (d, J=8.0 Hz, 11H), 6.50 (m, 1H), 6.82 (s, 1H), 6.95 (s, br, 4H), 7.15 (d, J=7.0 Hz, 2H), 7.25 (d, J=7.0 Hz, 2H).

### Step 8: 9a-(4-chlorobenzyl)-4-{4-[2-(1-piperidinyl)ethoxy]phenyl}-7-hydroxy-1,2,9,9a-tetrahydro-3H-fluoren-3-one

A solution of 9a-(4-chlorobenzyl)-7-methoxy-{4-[2-(1-piperidinyl)-ethoxy]phenyl}-1,2,9,9a-tetrahydro-3H-fluoren-3-one (96 mg, 0.177 mmol) in anhydrous CH₂Cl₂ (4 mL) was placed under a nitrogen atmosphere and cooled to 0° C. By a syringe a solution of AlCl₃ (215 mg, 1.61 mmol) in 2-propanethiol (2 mL) was added. The resulting mixture was stirred for 3 h at room. The solvents were removed by a stream of nitrogen and MeOH (1 mL) was added to the residue. The resulting mixture was partitioned between saturated aqueous NaHCO₃ and EtOAc. The organic phase was dried over NaSO₄, filtered and evaporated under vacuum to afford 9a-(4-chlorobenzyl)-7-hydroxy-4-{4-[2-1-piperidinyl)ethoxy]phenyl}-1,2,9,9a-tetrahydro-3H-fluoren-3-one as a yellow solid.

¹H NMR (DMSO-d₆, 400 MHz) δ 1.33-1.42 (m, 2H), 1.45-1.55 (m, 6H), 2.12 (m, 2H), 2.45 (m, 4H, in part covered by solvent signal), 2.60-2.70 (m, 4H), 2.73 (m, 1H), 2.85 (m, 1H), 2.97 (d, J=15 Hz, 2H), 4.10 (t, J=6.5 Hz, 2H), 6.12 (d, J=8.0 Hz, 1H), 6.30 (m, 1H), 6.61 (s, 1H), 6.95 (s, br, 4H), 7.14 (d, J=7.0 Hz, 2H), 7.24 (d, J=7.0 Hz, 2H).

MS: m/z (M+H⁺): 528.2.

### Example 3

### Synthesis of (3e)-9a-Benzyl-7-Hydroxy-4-Methyl-1,2,9,9a-Tetrahydro-3h-Fluoren-3-One Methoxime

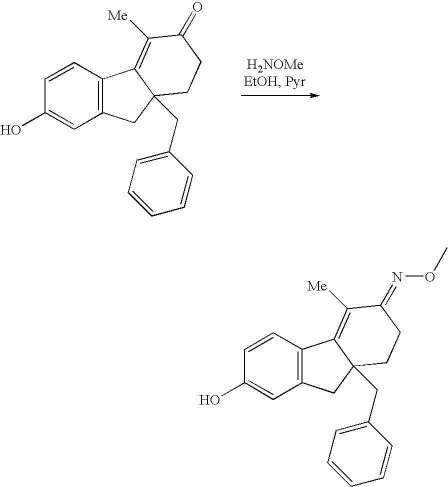

A solution of 9a-benzyl-7-hydroxy-4-methyl-1,2,9,9a-tetrahydro-3H-fluoren-3-one (50 mg, 0.16 mmol, prepared as described in example I) and O-methylhydroxylamine hydrochloride (27 mg, 0.32 mmol) in a 1:1-mixture of EtOH and anhydrous pyridine (0.5 mL) was stirred at room temperature for 16 hours. The reaction mixture was diluted with CH₂Cl₂, washed with 1N HCl and brine, dried over Na₂SO₄, filtered, and evaporated under vacuum. The title compound was obtained as a colourless solid (25 mg).

¹H NMR (DMSO-d₆, 400 MHz), δ 1.42-1.56 (m, 1H), 1.92 (m,1H), 2.09 (s, 3H), 2.34 (m, 2H), 2.53-2.70 (m, 2), 2.78-2.90 (m, 2H), 3.87 (s, 3H), 6.66 (in 2H), 7.04 (m, 2H), 7.14-7.26 (m, 3H), 7.43 (d, J=8.3 Hz, 1H).

### Example 4

### Synthesis of 6-Methyl-9a-(4-Fluorobenzyl)-8,9,9a,10-Tetrahydroindeno[2,1-E]Indazol-7(3h)-One

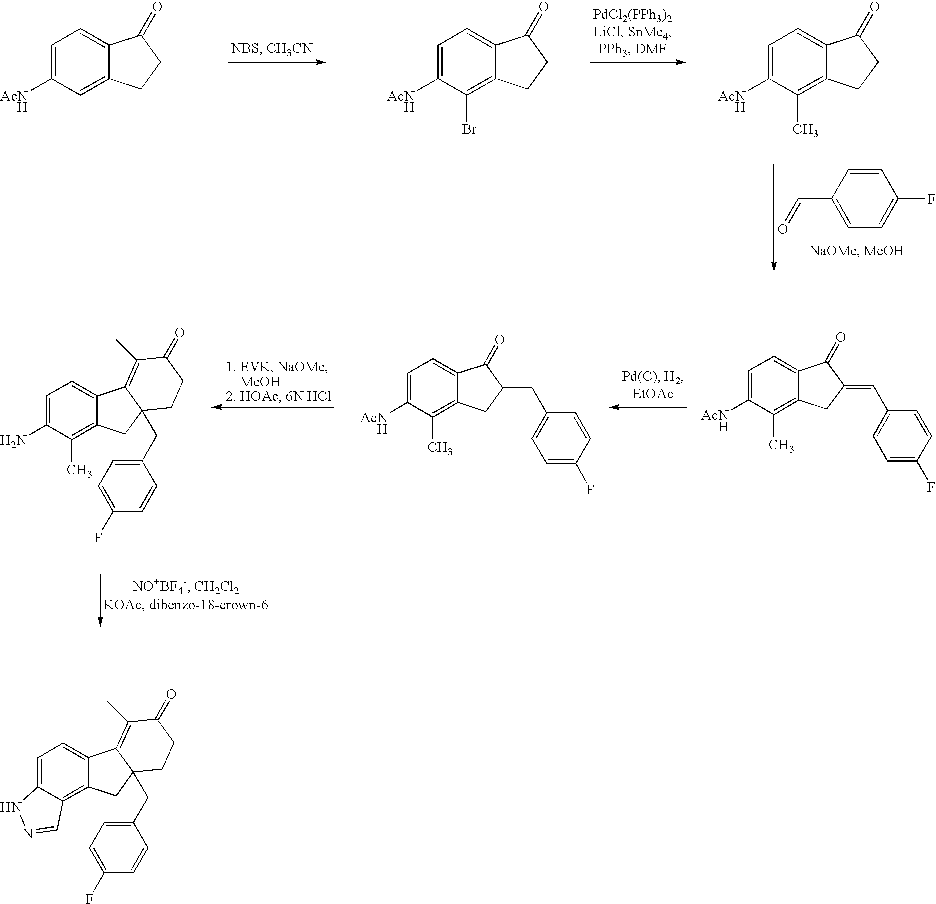

### Step 1: 5-(acetylamino)-4-bromo-1-indanone

A suspension of N-(1-oxo-2,3-dihydro-1H-inden-5-yl)acetamide (1.50 g, 7.93 mmol) and N-bromosuccinimide (1.47 g, 8.26 mmol) in 8 mL acetonitrile was stirred and heated to 60° C. for 7 h. After cooling to room temperature a formed precipitate was filtered off and dried under vacuum to afford the title compound (1.28 g).

¹H-NMR (DMSO-d₆, 400 MHz): δ 2.16 (s, 3H), 2.68 (m, 2H), 3.01 (m, 2H), 7.61 (d, J=8.0 Hz, 1H), 7.83 (d, J=8.0 Hz, 1H), 9.62 (s, 1H).

### Step 2: 5-acetylamino)-4-methyl-1-indanone

A suspension of 5-(acetylamino)-4-bromo-1-indanone (8.34 g, 31.1 mmol), dichlorobis(tripenylphosphine)palladium(II) (1.09 g, 1.38 mmol), tetramethyltin (4.3 mL, 1 eq.), triphenylphosphine (0.82 g, 1 eq.) and lithium chloride (2.63 g, 2 eq.) in 83 mL of anhydrous dimethylformamaide was stirred and heated to 100° C. under a nitrogen atmosphere for 6 h. After cooling to room temperature the mixture was filtered over celite and concentrated under reduced pressure. The residue was dissolved in CH₂Cl₂-5% MeOH and the solution was washed with brine. After concentration under reduced pressure the residue was triturated with diethyl ether and left under high vacuum to afford 4.6 g of the title compound.

¹H-NMR (DMSO d, 400 MHz): δ 2.11 (s, 3H), 2.20 (s, 3H), 2.62 (m, 2H), 3.00 (m, 2H), 7.43 (d, J=8.2 Hz, 1H), 7.59 (d, 3=8.2 Hz, 1H), 9.53 (s, 1H).

### Step 3: 5-(acetylamino)-2-(4-fluorobenzylidene)-4-methyl-1-indanone

To a solution of 5-acetylamino)-4-methyl-1-indanone (0.5 g, 2.46 mmol) and 4-fluorobenzaldehyde (1.2 eq.) in 10 mL of anhydrous EtOH were added dropwise under stirring 1.5 mL of 0.5M NaOMe/MeOH. The mixture was stirred for 16 h at room temperature and the formed precipitate was filtered off and washed with EtOH. After drying under vacuum 606 mg of the title compound were obtained.

¹H-NMR (DMSO-d₆, 400 MHz): δ 2.12 (s, 3H), 2.30 (s, 3H), 4.03 (s, 2H), 7.33 (m, 2H), 7.50 (s, 1H), 7.59 (d, J=8.0 Hz, 1H), 7.67 (d, J=8.0 Hz, 1H), 7.89 (r, 2H), 9.58 (s, 1H).

### Step 4: 5-(acetylamino)-2-(4-fluorobenzyl)-4-methyl-1-indanone

A suspension of the product from step 3 (651 mg, 2.1 mol) and palladium on charcoal (10%, 150 mg) in 60 mL EtOAc was stirred under a hydrogen atmosphere at 40° C. After 1.5 h the catalyst was filtered off and the solution was concentrated to dryness under reduced pressure to afford the title compound (438 mg).

¹H-NMR (DMSO-d₆, 300 MHz): δ 2.09 (s, 3H), 2.12 (s, 3H), 2.60-2.75 (m, 2H), 2.95-3.21 (m, 3H), 7.11 (m, 2H), 7.31 (m, 2H), 7.45 (d, J=8.1 Hz, 1H), 7.61 (d, J=8.1 Hz, 1H), 9.52 (s, 1H).

### Step 5: 7-amino-9a-(4-fluorobenzyl)-4,8-dimethyl-1,2,9,9a-tetrahydro-3H-fluoren-3-one

To a stirred suspension of 5-(acetylamino)-2-(4-fluorobenzyl)-4-methyl-1-indanone (438 mg, 1.41 mmol) and ethyl vinyl ketone (1.25 eq.) in 2.8 mL anhydrous methanol were added dropwise a solution of sodium methoxide (0.5 M) in methanol (0.5 mL). The mixture was stirred and heated to 60° C. for 3 hours. After cooling to room temperature the mixture was concentrated under reduced pressure. The residue was dilutesd with ethyl acetate and washed with water and brine. The organic phase was dried over Na₂SO₄, filtered and concentrated under reduced pressure. An oily material was obtained, which was dissolved in a mixture of 10 mL HOAc and 10 mL 6 N HCl. The mixture stirred and heated to 80° C. for 3 h. After cooling to room temperature the mixture was neutralized with saturated aq. NaHCO₃ and extracted with ethyl acetate. The organic phase was dried over Na₂SO₄, filtered and concentrated under reduced pressure to obtain the title compound as a yellow foam (198 mg).

¹H-NMR (CDCl₃, 300 MHz): δ 1.94-2.03 (m, 1H), 2.07 (s, 3H), 2.12 (s, 3H), 2.14-2.23 (m, 1H), 2.43 (d, J=15.9 Hz, 1H), 2.50-2.69 (m, 2H), 2.72-2.83 (m, 1H), 2.85 (d, J=13.8 Hz, 1H), 3.02 (d, J=15.9 Hz, 1H), 3.94 (s, br, 2H), 6.63 (d, J=8.2 Hz, 1H), 6.86-7.04 (m, 4H), 7.43 (d, J=8.2 Hz, 1H).

### Step 6: 6-methyl-9a-(4-fluorobenzyl)-8,9,9a,10-tetrahydroindeno[2,1-e]indazol-7(3H)-one

To a solution of 7-amino-9a-(4-fluorobenzyl)-4,8-dimethyl-1,2,9,9a-tetrahydro-3H-fluoren-3-one (196 mg, 0.59 mmol) in CH₂Cl₂ (5 mL) at −35° C. was added potassium tetrafluoroborate (69 mg, 0.59 mmol). The mixture was stirred for one hour during which the temperature was left reaching 4° C. The mixture was again cooled to −35° C., diluted with CH₂Cl₂ (5 mL) and potassium acetate (122 mg, 1.24 mmol) and dibenzo-18-crown-6 (8 mg, 22 mol) were added. The cooling bath was removed and the mixture was stirred for 1 hour at room temperature. The mixture was then partitioned between CH₂Cl₂ and water. The organic phase was washed with brine, dried over Na₂SO₄, filtered and concentrated under reduced pressure. The crude product was purified by flash chromatography on silicagel (eluent: CH₂Cl₂/EtOAc 9:1, v/v), The title compound was obtained as an orange solid (78 mg).

MS: m/z 347.0 for [M+H]⁺

¹H-NMR (DMSO-d₆, 300 MHz): δ 2.07 (s, 3H), 2.1-2.2 (m, 2H), 2.33-2.45 (m, 1H), 2.63 (d, J=13.4 Hz, 1H), 2.72-2.95 (m, 3H), 3.32 (d, 1H, in part overlaid by water signal), 6.83-7.12 (m, 4H), 7.46 (d, J=8.7 Hz, 1H), 7.69 (d, J=8.7 Hz, 1H), 8.15 (s, 1H), 13.27 (s, 1H).

### Example 5

### Synthesis of 9a-Benzyl-6-{4-[2-(1-Piperidinyl)Ethoxy]Phenyl}-8,9,9a 10-Tetrahydroindeno[2,1-E]Indazol-7(3h)-One Hydrotrifluoroacetae Salt (CMP9)

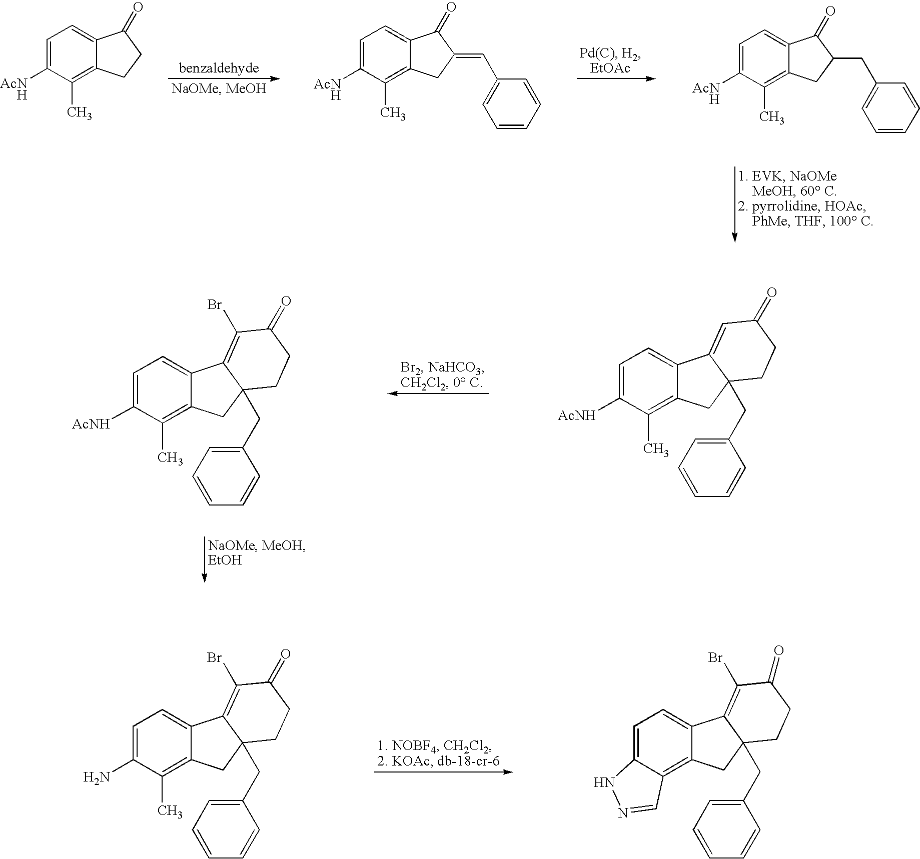

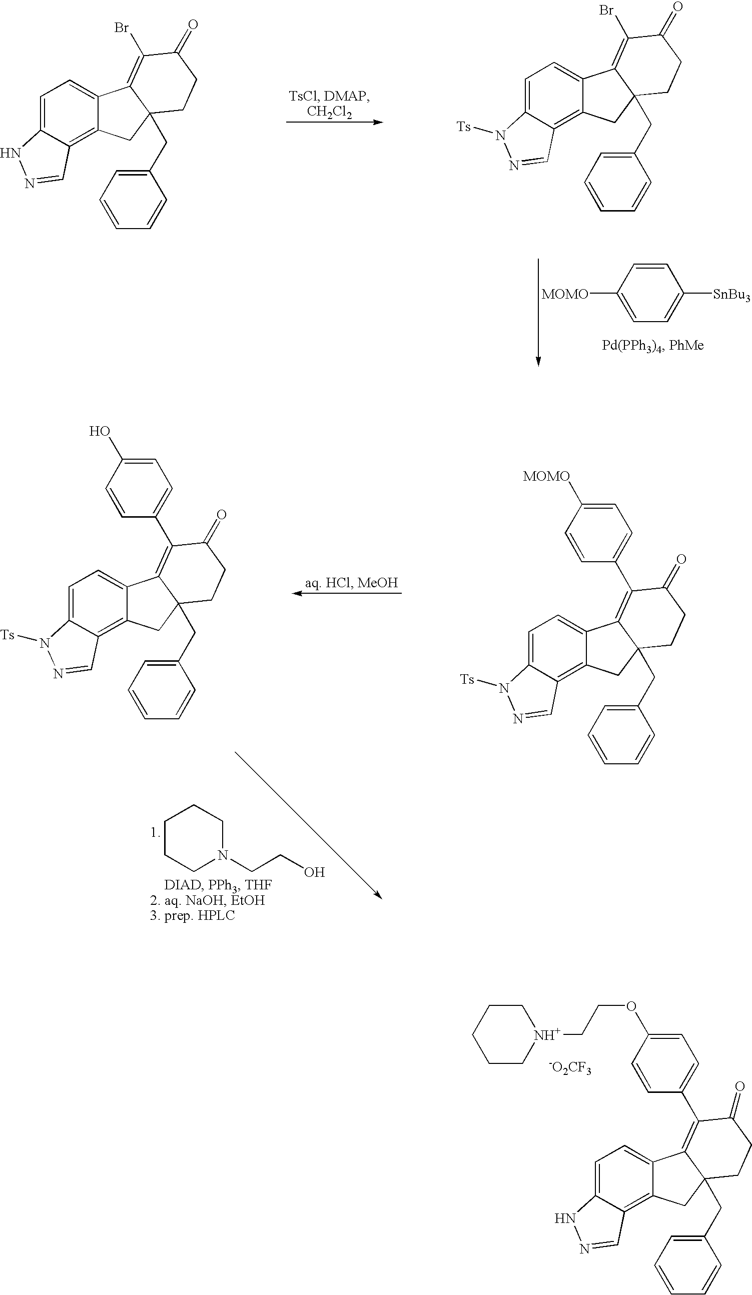

### Step 1: 5-(Acetylamino)-2-benzylidene-4-methyl-1-indanone

To a suspension of 5-acetylamino)-4-methyl-1-indanone (2.0 g, 9.84 mmol), prepared as described in example 5, and benzaldehyde (11.9 mmol) in 40 mL of anhydrous EtOH were added dropwise under stirring 6.0 mL of 0.5M NaOMe/MeOH. The mixture was stirred for 6 h at room temperature and the formed precipitate was filtered off and washed with EtOH. After drying under vacuum 2.11 g of the title compound as a yellow solid were obtained.

¹H-NMR (DMSO-d₆, 400 MHz): δ 2.14 (s, 3H), 2.30 (s, 3H), 4.32 (m, 2H), 7.42 (m, 4H), 7.60 (d, J=8.0 Hz, 1H), 7.68 (d, J=8.0 Hz, 1H), 7.73 (m, 2H), 9.58 (s, 1H).

### Step 2: 5-(Acetylamino)-2-benzyl-4-methyl-1-indanone

A suspension of the product from step 3 (2.11 g, 7.25 mol) and palladium on charcoal (10%, 200 mg) in a mixture of 40 mL EtOAc and 40 mL MeOH was stirred under a hydrogen atmosphere at 40° C. After 1.5 h the catalyst was filtered off and the solution was concentrated to dryness under reduced pressure. After flash chromatography on silicagel (eluent: CH₂Cl₂/EtOAc 9:1, v/v) the title compound was obtained as a white solid (1.64 g).

¹H-NMR (DMSO-d₆, 400 MHz): δ 2.10 (s, 3H), 2.12 (s, 3H), 2.60-2.75 (m, 2H), 2.97-3.12 (m, 2H), 3.13-3.22 (m, 1H), 7.15-7.32 (m, 5H), 7.46 (d, J=7.8H-z, 1H), 7.60(d, J=7.8 Hz, 1H), 9.52 (s, 1H).

### Step 3: 7-(Acetylamino)-9a-benzyl-8-methyl-1,2,9,9a-tetrahydro-3H-fluoren-3-one

To a stirred solution of 5-(acetylamino)-2-benzyl-4-methyl-1-indanone (1.64 g, 5.60 mmol) and methyl vinyl ketone (7.06 mmol) in 14 mL anhydrous methanol were added dropwise a solution of sodium methoxide in methanol (0.5 M, 2.3 mL). The mixture was stirred at room temperature for 19 hours. It was then concentrated under reduced pressure and the residue partitioned between ethyl acetate and brine. The aqueous phase was extracted with ethyl aceate and the combined organic phases were dried over Na₂SO₄, filtered and concentrated under reduced pressure. An oily material was obtained, which was dissolved in a mixture of 51 mL THF, 4.4 mL toluene, 0.5 mL pyrrolidine and 0.27 mL HOAc. The mixture stirred and heated to 100° C. for 75 min. After cooling to room temperature the mixture was concentrated under reduced pressure. After flash chromatography on silicagel (eluent: CH₂Cl₂/EtOAc 8:2, v/v) 1.18 g of the title compound was obtained.

¹H-NMR (DMSO₆, 400 MHz): δ 1.92-2.03 (m, 1H), 2.05-2.13 (m, 6H), 2.13-2.19 (m, 1H), 2.38-2.63 (m, 3H, in part under solvent signal), 2.73-2.86 (m, 1H), 2.93 (d, J=13.3 A, 1H), 3.10 (d, J=15.7 Hz, 1H), 6.28 (s, 1H), 7.10-7.26 (m, 5H), 7.37-7.53 (m, 2H), 9.39 (s, 1H).

### Step 4: 7-(Acetylamino)-4-bromo-9a-benzyl-8-methyl-1,2,9,9a-tetrahydro-3H-fluoren-3-one

To a stirred suspension of 7-amino-9a-benzyl-8-methyl-1,2,9,9a-tetrahydro-3H-fluoren-3-one (421 mg, 1.2 mmol) and NaHCO₃ (500 mg) in CH₂Cl₂ (11 mL) at 0° C. was added dropwise a solution of bromine (1 eq.). The mixture was stirred for additional 15 min at 0° C. and then diluted with water (40 mL). The organic phase was separated, dried over Na₂SO₄ and concentrated under reduced pressure to afford the title compound (595 mg).

¹H-NMR (DMSO-d₆, 400 MHz): δ 2.02-2.20 (m, 8H), 2.55-2.67 (m, 3H), 2.93-3.13 (m, 3H), 7.03-7.22 (m, 5H), 7.55 (d, J=7.3 Hz, 1H), 8.18 (d, J=7.3 Hz, 1H), 9.42 (s, 1H).

### Step 5: 7-amino-4-bromo-9a-benzyl-8-methyl-1,2,9,9a-tetrahydro-3H-fluoren-3-one

A solution of 7-(acetylamino)-4-bromo-9a-benzyl-8-methyl-1,2,9,9a-tetrahydro-3H-fluoren-3-one (1.56 g, 3.41 mmol) in EtOH (22 mL) and sodium methoxide in methanol (0.5 M, 21 mL) under nitrogen was stirred and heated to 80° C. for 6.5 h.

After cooling to room temperature the mixture was partitioned between ethylacetate and brine. The organic phase was dried over Na₂SO₄, filtered and concentrated under reduced pressure to obtain the title compound as a yellow foam (983 mg).

¹H-NMR (DMSO-d₆, 400 MHz): δ 1.95-2.10 (m, 5H), 2.40-2.60 (m, 3H, in part under solvent signal), 2.92-3.09 (m, 3H), 5.89 (s, br, 2H), 6.60 (d, J=7.9 Hz, 1H), 7.03-7.26 (m, 5H), 8.02 (d, J=7.9 Hz, 1H).

### Step 6: 6-bromo-9a-benzyl-8,9,9a,10-tetrahydroindeno[2,1-e]indazol-7(3H)-one

To a solution of 7-amino-4-bromo-9a-benzyl-8-methyl-1,2,9,9a-tetrahydro-3H-fluoren-3-one (983 mg, 2.57 mmol) in CH₂Cl₂ (14 mL) at −35° C. was added nitrosonium tetrafluoroborate (304 mg, 2.60 mmol). Stirring was continued for 70 min while the temperature was allowed to reach 0° C. The solution was cooled again to −35° C. and KOAc (509 mg, 5.2 mmol) and dibenzo-18-crown-6 (48 mg) were added. The cooling bath was removed and the stirring was continued for 2 h at room temperature. The solution was diluted with CH₂Cl₂ (100 mL) and washed with water and brine. The organic phase was dried over Na₂SO₄, filtered and concentrated under reduced pressure. After flash chromatography on silicagel (eluent: CH₂Cl₂/EtOAc 9:1, v/v) 353 mg of the title compound were obtained as a brown-red solid.

¹H-NMR (DMSO-d₄, 300 MHz): δ 2.1-2.3 (m, 2H), 2.67-2.78 (m, 2H), 2.90-3.20 (m, 3H), 3.45 (d, J=16.7 Hz, 1H), 6.90-7.15 (m, 5H), 7.50 (d, J=9.3 Hz, 1H), 8.11 (s, 1H), 8.39 (d, J=9.3 Hz, 1H), 13.40 (s, 1H).

### Step 7: 6-bromo-9a-benzol-3-[(4-methylphenyl)sulfonyl]-8,9,9a,10-tetrahydroindeno-[2,1-e]indazol-7(3H)-one

A solution of 6-bromo-9a-benzyl-8,9,9a, 10-tetrahydroindeno[2,1-e]indazol-7(3H)-one (353 mg, 0.90 mmol), 4-(dimethylamino)-pyridine (165 mg, 1.35 mmol) and p-toluenesulfonyl chloride (207 mg, 1.09 mmol) in 5 mL anhydrous CH₂Cl₂ was stirred under nitrogen for 1 h at 0° C. and 2 h at room temperature. After dilution with CH₂Cl₂ the solution was washed sequentially with water, phosphate buffer (1M, pH 3), 5% aq. NaHCO₃ and brine. The organic phase was dried over Na₂SO₄, filtered and concentrated under reduced pressure. After flash chromatography on silicagel (eluent: petroleum ether/EtOAc 7:3, v/v) 156 mg of the title compound were obtained. (The 2-[(4-methylphenyl)sulfonyl]-isomer (200 mg) was eluted before the title compound).

¹H-NMR (CDCl₃, 400 MHz): δ 2.14-2.25 (m, 1H), 2.30-2.48 (m, 1H), 2.41 (s, 3H), 2.74-3.10 (m, 5H), 3.40 (d, J=16.8 Hz, 1H), 6.90-7.13 (m, 5H), 7.31 (d, J=8.2 Hz, 1H), 7.92 (d, J=8.2 Hz, 1H), 8.15 (s, 1H), 8.18 (d, J=9.1, 1H), 8.72 (d, J=9.1, 1H).

### Step 8: 9a-benzyl-6-[4-(methoxymethoxy)phenyl]-3-[(4-methylphenyl)sulfonyl]-8,9,9a,10-tetrahydroindeno-[2,1-e]indazol-7(3H)-one

A solution of 6-bromo-9a-benzyl-3-[(4-methylphenyl)sulfonyl]-8,9,9a,10-tetrahydroindeno-[2,1-e]indazol-7(3H)-one (156 mg, 0.34 mmol), Pd(PPh₃)₄ (18 mg, 15.5 μmol), and tributyl-(4-methoxymethoxy-phenyl)-stannane (160 mg, 0.37 mmol) in anhydrous toluene (2.5 mL) was purged with nitrogen and heated with stirring to 100° C. under nitrogen for 24 h. The mixture was cooled to room temperature and concentrated under vacuum. After flash chromatography on silicagel (eluent: petroleum ether/EtOAc 1:1, v/v) 137 mg of the title compound were obtained.

¹H-NMR (CDCl₃, 400 MHz): δ 2.20-2.31 (m, 1H), 2.35-2.45 (m, 4H), 2.69-2.77 (m, 1H), 2.80-2.88 (m, 2H), 2.91-3.08 (m, 2H), 3.40 (d, J=16.8 Hz, 1H), 3.60 (s, 3H), 5.29 (m, 2H), 6.61 (d, J=8.8 Hz, 1H), 6.90-7.30 (m, 11H), 7.74 (d, J=8.8 Hz, 1H), 7.87 (d, J=8.2 Hz, 2H), 8.12 (s, 1H).

### Step 9: 9a-benzyl-6-(4-hydroxyphenyl)-3-[(4-methylphenyl)sulfonyl]-8,9,9a,10-tetrahydroindeno-[2,1-e]indazol-7(3H)-one

A solution of 9a-benzyl-6-[4-(methoxymethoxy)phenyl]-3-[(4-methylphenyl)sulfonyl]-8,9,9a,10-tetrahydroindeno-[2,1-e]indazol-7(3H)-one (137 mg, 0.226 mmol) in methanol (5.2 mL) and aqueous 2N HCl (0.52 mL) was stirred and heated to 80° C. for 1 h. After cooling to room temperature and the mixture was concentrated under reduced pressure. The residue was partitioned between EtOAc and water and the organic phase was washed with brine. It was then dried over Na₂SO₄, filtered and concentrated under vacuum to leave 124 mg of the title compound as a yellow foam.

¹H NMR (CDCl₃, 400 MHz) δ 2.20-2.30 (m, 1H), 2.34-2.45 (m, 4H), 2.69-2.77 (m, 1H), 2.79-2.87 (m, 2H), 2.91-3.08 (m, 2H), 3.40 (d, J=16.8 Hz, 1H), 6.61 (d, J=8.8 Hz, 1H), 6.80-7.32 (m, 11H), 7.73 (d, J=8.8 Hz, 1H), 7.85 (d, J=8.2 Hz, 2H), 8.11 (s, 1H).

### Step 10: 9a-benzyl-6-{4-[2-(1-piperidinyl)ethoxy]phenyl}-8,9,9a,10 tetrahydroindeno-[2,1-e]indazol-7(3H)-one hydrotrifluoroacetate salt

To a solution of 9a-benzyl-6-(4-hydroxyphenyl)-3-[(4-methylphenyl)sulfonyl]-8,9,9a,10-tetrahydroindeno-[2,1e]indazol-7(3H)-one (124 mg, 0.22 mmol), triphenylphosphine (175 mg, 0.66 mmol) and piperidineethanol (88 μL, 0.22 mmol) in 2.0 mL of anhydrous tetrahydrofurane at 0° C. under nitrogen was added dropwise a solution of diisopropyl-azodicarboxylate (140 μL, 0.72 mmol) in anhydrous tetrahydrofurane (500 μL). The solution was stirred for 1 h at 0° C. and for 5 h at room temperature. The product was purified by flash chromatography on silica gel, using CH₂Cl₂-5 vol % MeOH as eluent After removal of the solvents under reduced pressure the crude (4-methylphenyl)sulfonyl-protected intermediate was obtained as a yellow foam, which was dissolved in a mixture of 2 mL EtOH, 2 mL 1,4-dioxane and 1 mL 1N aqueous NaOH. The mixture was stirred for 3 h at room temperature, acidified with HOAc and the solvents removed under reduced pressure. The residue was partitioned between CH₂Cl₂ and brine. The organic phase was dried over Na₂SO₄, filtered and concentrated under vacuum. The crude product was purified by preparative RP-HPLC, using water (0.1% TFA) and acetonitrile (0.1% TFA) as eluents (column: Waters SymmetryPrep C18, 22×100 mm). The pooled product fractions were lyophilized to afford 25 mg of the title compound.

MS: m/z 518.2 for [M+H]⁺

¹H-NMR (DMSO-d₆, 400 MHz): δ 1.33-1.50 (m, 1H), 1.64-1.90 (m, 5H), 2.18-2.30 (m, 2H), 2.40-2.50 (m, 1H, in part under solvent signal), 2.79 (d, J=12.8 Hz, 1H), 2.83-3.10 (m, 6H), 3.50-3.62 (m, 4H, in part under water signal), 4.41 (m, 2H), 6.30 (d, J=8.4 Hz, 1H), 6.94-7.25 (m, 10H), 8.13 (s, 1H), 9.38 (s, br, 1H).

### Example 6

### Synthesis of 9a-(4-Fluorobenzyl)-6-Methyl-8,9,9a,10-Tetrahdrofluoreno[1,2-D][1,2,3]Triazol-7(3h)-One (L884,653)

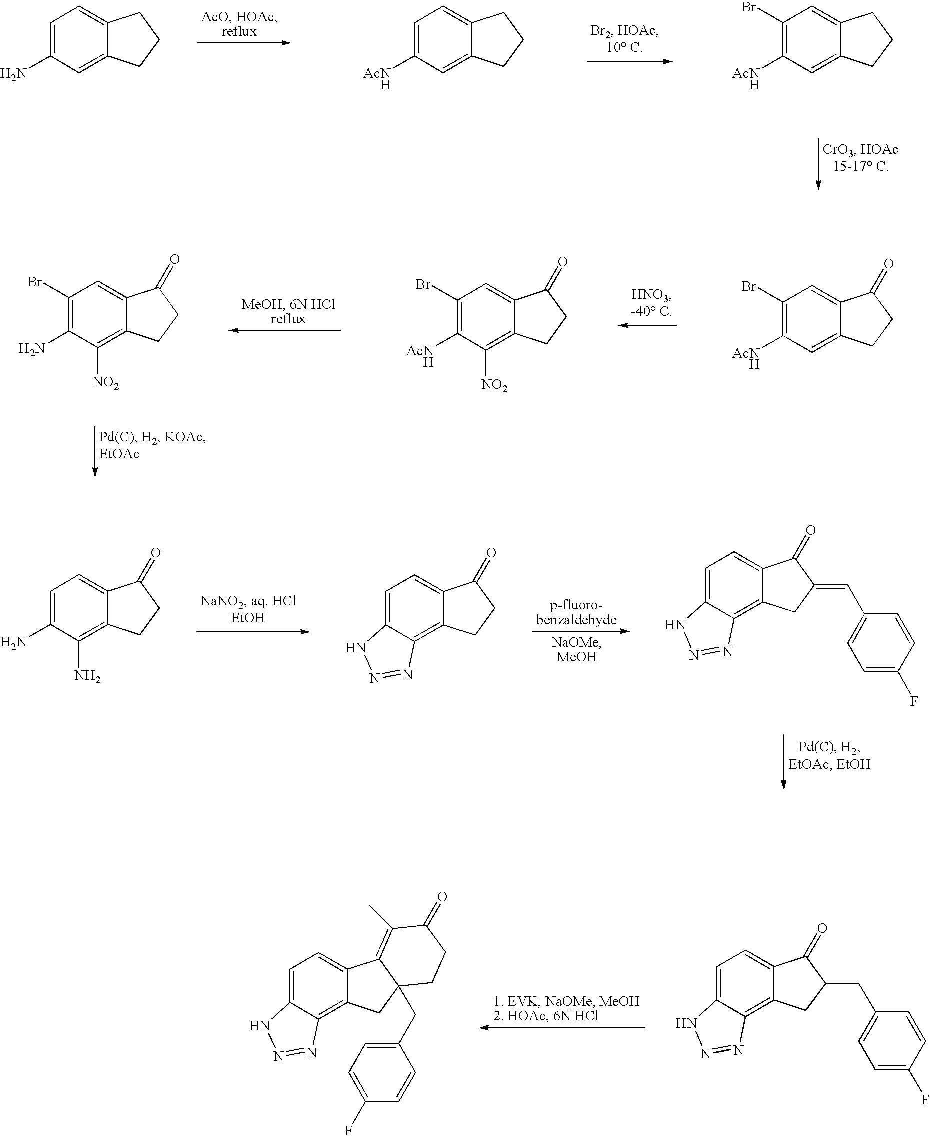

### Step 1: 5-acetamido-indane

A solution of 5-aminoindane (30 g, 225.2 mmol) in acetic acid (75 mL) and acetic anhydride (55 mL) was refluxed for 1.5 h and the solvents were distilled off under reduced pressure. A brown oil remained which solidified upon standing at room temperature. The crude material (39.8 g) was dissolved in methanol (100 mL) and 1M aqueous K₂CO₃ (35 mL) was added. The solution was left standing at room temperature for 30 minutes and then partitioned between CH₂Cl₂ (500 ml) and water (400 mL). The organic phase was dried over Na₂SO₄, filtered and concentrated under reduced pressure to afford 33.5 g of the title compound as a light brown solid.

¹H-NMR (CDCl₃, 400 MHz): δ 7.45 (s, 1H), 7.15 (s, 3H), 2.88 (m, 4H), 2.17 (s, 3H), 2.08 (m, 2H).

### Step 2: 6-Bromo-5-acetamido-indane

To a solution of 5-acetamido-indane (10 g, 57.1 mmol) in glacial acetic acid (170 mL) cooled to ca. 10° C. was added bromine (3.6 mL) over a period of 1 h, maintaining the temperature around 10° C. After stirring for an additional 10 minutes at 10° C., the mixture was diluted with water (600 mL). Stirring was continued until a solid precipitate had formed (15 min). The precipitate was filtered off and dissolved in CH₂Cl₂ (100 mL). A residual water phase was separated and the organic phase was dried over Na₂SO₄, filtered and concentrated to afford 12.45 g of the title compound as light yellow solid.

¹H-NMR (CDCl₃, 400 MHz): δ 8.14 (s, 1H), 7.52 (s, br, 1H), 7.38 (s, 1H), 2.88 (m, 4H), 2.10 (m, 2H).

### Step 3: 6-Bromo-5-acetamido-indan-1-one

To a stirred solution of 6-bromo-5-acetamido-indane (12.45 g, 49 mmol) in acetic acid (170 mL) cooled to 15° C. was added dropwise a solution of CrO₃ (16.7 g, 167 mmol) in water (33 mL) over 25 minutes, keeping the temperature between 15-17° C. The mixture was stirred for an additional 90 min. at 16-18° C. and then water (520 mL) was added. Stirring was continued for 30 minutes at 5° C. The formed precipitate was filtered off, washed with cold water and dried under high vacuum for 16 hours. The title compound was obtained as a light yellow solid (8.0 g).

¹H-NMR (CDCl₃, 400 MHz): δ, 8.60 (s,1H), 7.94 (m, 2H), 3.10 (m, 2H), 2.71 (m, 2H), 2.31 (s, 3H).

### Step 4: 6-Bromo-5-acetamido-4-nitro-indan-1-one

Nitric acid (65 mL) was cooled to −40° C. Then 6-bromo-5-acetamido-indan-1-one (8.0 g, 29.8 mmol) was added portionwise over a period of 2 min. The resulting solution was stirred for 50 minutes, during which the temperature was allowed to raise gradually to −20° C. The reaction mixture was poured into water (1.1 L). The formed precipitate was filtered off, washed with water, dried first under air stream and then under high vacuum for 16 h. The title compound was obtained as a light yellow solid (7.5 g).

¹H-NMR (DMSO-d₆, 400 MHz): δ 10.43 (s, 1H), 8.19 (s, M1), 3.18 (m, 2H), 2.73 (m, 2H), 2.09 (s, 3H).

### Step 5: 6-Bromo-5-amino-4-nitro-indan-1-one

A suspension of 6-bromo-5-acetamido-4-nitro-indan-1-one (3 g, 9.6 mmol) in methanol (150 mL) and aqueous HCl (120 mL, 6.25 N) was refluxed for 2.5. After cooling to room temperature the mixture was poured into water (1.8 L) and stirred for 5 minutes at room temperature. The formed precipitate was filtered off and washed with water (200 mL). After drying for 24 hours under high vacuum 1.9 g of the title compound were obtained as an orange solid.

¹H-NMR (DMSO-d₆, 400 MHz): δ 7.95 (s, 1H), 7.79 (s, 3H), 3.30 (m, 2H), 2.55 (m, 2H).

### Step 6: 4,5-Diamino-indan-1-one

A suspension of 6-Bromo-5-amino-4-nitro-indan-1-one (13.9 g, 51 mmol), NaOAc (32 g) and palladium on charcoal (10%, 2.55 g) in 1 L anhydrous EtOAc was stirred under a hydrogen atmosphere at atmospheric pressure for 24 hours. Further catalyst (1 g) was added and stirring was continued for 24 hours. The catalyst was filtered off and washed with EtOH (800 mL). The combined organic phases were concentrated to ca 50 mL and partitioned between CH₂Cl₂ (500 mL) and 5% NaHCO₃ (500 mL). The organic phase was dried over Na₂SO₄, filtered and concentrated to dryness. The title compound was obtained an orange solid (5.3 g).

¹H-NMR (DMSO-d₆, 400 MHz): δ 6.80 (d, J=10.0 Hz, 1H), 6.55 (d, J=10.0 Hz, 1H), 5.45 (s, 2H), 4.53 (s, 2H), 2.70(m, 2H), 2.49(partially covered by DMSO, m, 2H).

### Step 7: 7,8-Dihydroindeno[4,5-d][1,2,3]triazol-6(3H)-one

Under warming 4,5-diaminoindan-1-one (1.0 g, 6.2 mmol) were dissolved in 80 ml EtOH. After cooling to room temperature 6.1 mL of 37% HCl and 1.5 mL of water were added. The solution was cooled to 0° C. and a solution of 1.38 g Na NO₂ in 6.5 mL water were added dropwise. The resulting dark brown mixture was stirred for further 50 minutes at ca 5° C. The mixture was partitioned between 500 mL EtOAc and 400 mL water. The organic phase was washed with 300 mL brine, dried over NaSO₄ and the solvent was distilled off under vacuum. The product was purified by flash chromatography on a silicagel, using EtOAc/petrolether (1:1, v/v) as eluent. The title compound was obtained as an orange solid (632 mg).

MS: m/z 174 for [M+H]⁺.

¹H-NMR: (DMSO-d₆, 400 MHz) 8 (ppm) 7.85 (d, 8.5 Hz, 1H), 7.64 (d, 8.5 Hz, 1H), 3.45 (ma, 2H), 2.77 (m, 2H).

### Step 8: 7-(4-Fluoro-benzylidene)-7,8-dihydroindeno[4,5-d][1,2,3]triazol-6(3H)-one

7,8-Dihydroindeno[4,5-d][1,2,3]triazol-6(3H)-one (200 mg, 1.16 mmol) were dissolved in 17 ml EtOH under warming. After cooling to room temperature 139 μL (1.5 eq.) of 4-fluoro-benzaldehyde and 5.0 mL of 0.5 M NaOMe in MeOH (1.5 eq.) were added. The reaction mixture was stirred at room temperature for 17 h. Under stirring 3.7 mL of 1 N HCl and 200 mL of water were added. The formed precipitate was filtered off and dried under high vacuum 260 mg of the title compound were obtained.

MS: m/z 280 for [M+H]⁺.

¹H-NMR (DMSO-d₆, 400 MHz): δ (ppm) 16.3 (s, br, ca.1H), 8.00-7.85 (m, 3H), 7.81 (m, 1H), 7.61 (s, 1H), 7.39 (m, 2H), 4.49 (s, 2H).

### Step 9: 7-(4-Fluoro-benzyl)-7,8-dihydroindeno[4,5-d][1,2,3]triazol-6(3H)-one

7-(4-Fluoro-benzylidene)-7,8-dihydroindeno[4,5-d][1,2,3]triazol-6(3H)-one (239 mg, 0.86 mmol) were dissolved 30 ml EtOAc-EtOH (1:1, v/v) under warming. After cooling to room temperature 48 mg of Pd/C (10%) were added. The reaction mixture was stirred under hydrogen at atmospheric pressure for 6 h at 45° C. The catalyst was filtered off and the solvent was distilled off under reduced pressure. 7-(4-Fluoro-benzyl)-7,8-dihydroindeno[4,5-d][1,2,3]triazol-6(3H)-one (253 mg) was obtained as a colourless oil and was used in the next step without further purification.

MS: m/z 282 for [M+H]⁺.

### Step 10: 9a-(4-Fluoro-benzyl)-6-methyl-8,9,9a,10-tetrahydrofluoreno[1,2d][1,2,3]-triazol-7(3H)-one

7-(4-Fluoro-benzyl)-7,8-dihydroindeno[4,5-d][1,2,3]triazol-6(3H)-one (253 mg, 0.86 mmol) were dissolved in 3 mL of 0.5M NaOMe/MeOH. EVK (127 μL, 1.5 eq.) was added, the mixture was heated to 60° C. for 5 h. The reaction mixture was cooled to room temperature and partitioned between 150 mL EtOAc and 100 mL 1 M aqueous KH₂PO₄. The organic phase was dried over NaSO₄, filtered and the solvent was distilled off under reduced pressure. An oily material was obtained, which was dissolved in a mixture of 5 mL HOAc and 5 mL 6 N HCL The mixture stirred and heated to 100° C. for 1 h. After cooling to room temperature and the mixture was partitioned between EtOAC and mL 1 M aqueous KH₂PO₄. The organic phase was dried over Na₂SO₄ and filtered. After removal of the solvent by distillation under reduced pressure the crude product was purified flash-chromatography on silicagel (eluent: EtOAc/petrol ether 1:2, v/v). The pooled product fractions were dissolved in 10 mL of 0.5M NaOMe/MeOH and the mixture was stirred and heated to 50° C. for 40 min. After cooling to room temperature and the mixture was partitioned between EtOAC and mL 1 M aqueous KH₂PO₄. The organic phase was dried over Na₂SO₄ and filtered. After removal of the solvent by distillation under reduced pressure the crude product was dissolved methyl-ter butyl ether (10 mL) and precipitated by addition of petroleum ether (35 mL). The precipitate was washed with petroleum ether and dried under high vacuum to afford 9a-(4-fluoro-benzyl)-6-methyl-8,9,9a,10-tetrahydro-fluoreno[1,2d]-[1,2,3]-triazol-7(3H)-one (38 mg) as a pale yellow solid.

MS: m/z 348.0 for [M+H]⁺,

¹H-NMR (MeOD-d₄, 400 MHz): δ 2.21 (s, 3H), 2.22-2.30 (m, 1H), 2.33-2.40 (m, 1H), 2.53-2.62 (m, 1H), 2.85-2.98 (m, 3H), 3.02 (d, J=16.8 Hz, 1H), 3.58 (d, J=16.8 Hz, 1H), 6.74 (m, 2H), 7.01 (m, 2H), 7.73 (d, J=8.4 Hz, 1H), 7.84 (d, J=8.4 Hz, 1H).

The following compounds were prepared using methods analogous to those described in the preceding examples:

TABLE Ia
(antagonists):

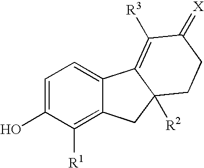

| | | | | | | GST- | GST- | | | |
|---|---|---|---|---|---|---|---|---|---|---|
| | | | | | | ERα | ERα | | | |
| | | | | | | MG- | wt- | | | |
| | | | | | | LBD | LBD | hERα | hERβ | |
| | | | | | | IC50 | IC50 | IC50 | IC50 | |
| CMP | R1 | R2 | R3 | X | MS | [nM] | [nM] | [nM] | [nM] | Name |
| 6 | H | benzyl | 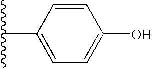 | O | 383.2 | 29 | 1371 | 223 | 96 | 9a-benzyl-7-hydroxy-4-(4-hy- droxyphenyl)-1,2,9,9a-tetra- hydro-3H-flour- en-3-one |
| 7 | H | benzyl | n-butyl | O | 347.2 | 25 | 1306 | 259 | 307 | 9a-benzyl-4-butyl-7-hy- |
| | | | | | | | | | | droxy-1,2,9,9a-tetra- |
| | | | | | | | | | | hydro-3H-fluor- |
| | | | | | | | | | | en-3-one |
| 8 | H | 4-chloro- benzyl | 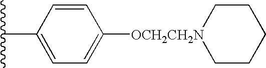 | O | 528.2 | 29 | 4436 | 1085 | 2232 | 9a-(4-chlorobenzyl)-7-hy- droxy-4-{4-[2-(1-pipe- ridinyl)-eth- oxy]phenyl}-1,2,9,9a-tetra- hydro-3H-fluor- en-3-one |
| 10 | H | benzyl | 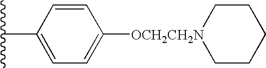 | O | 494.4 | 22 | 1272 | 375 | 184 | 9a-benzyl-7-hy- droxy-4-{4-[2-(1-pipe- ridinyl)-eth- oxy]phenyl}-1,2,9,9a-tetra- hydro-3H-fluor- en-3-one |
| 11 | H | benzyl | 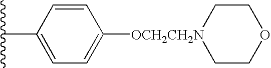 | O | 495.8 | 25 | 2308 | 706 | 231 | 9a-benzyl-7-hy- droxy-4-{4-[2-(4-mor- pholinyl)-eth- oxy]phenyl}-1,2,9,9a-tetra- hydro-3H-fluor- en-3-one |
| 12 | H | benzyl | 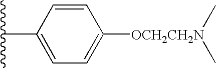 | O | 453.8 | 40 | 4171 | 1196 | 555 | 9a-benzyl-4-{4-[2-(di- methylamino)ethoxy]phe- nyl}-7-hydroxy-1,2,9,9a-tetra- hydro-3H-fluor- en-3-one |
| 13 | H | benzyl | 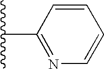 | O | 367.6 | 2022 | NA at 3600 nM | n.d. | n.d. | 9a-benzyl-7-hydroxy-4-py- ridin-2-yl-1,2,9,9a-tetra- hydro-3H-fluor- en-3-one |
| 14 | H | benzyl | 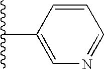 | O | 368.0 | 471 | NA at 3600 nM | n.d. | n.d. | 9a-benzyl-7-hydroxy-4-py- ridin-3-yl-1,2,9,9a-tetra- hydro-3H-fluor- en-3-one |
| 15 | H | benzyl | 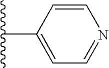 | O | 368.0 | 79 | 725 | 1182 | 558 | 9a-benzyl-7-hydroxy-4-py- ridin-4-yl-1,2,9,9a-tetra- hydro-3H-fluor- en-3-one |

TABLE 1b
(agonists):

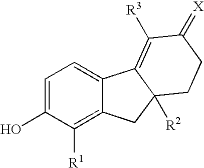

| | | | | | | GST-ERα | GST-ERα | hERα | hERβ | |
|---|---|---|---|---|---|---|---|---|---|---|
| | | | | | | MG-LBD | wt-LBD | IC50 | IC50 | |
| CMP | R1 | R2 | R3 | X | MS | IC50 [nM] | IC50 [nM] | [nM] | [nM] | Name |
| 4 | H | benzyl | Me | O | n.d. | 13.7 | 2320 | 1470 | 506 | 9a-benzyl-7-hy- |
| | | | | | | | | | | droxy-4-methyl-1,2,9,9a-tetra- |
| | | | | | | | | | | hydro-3H-fluor- |
| | | | | | | | | | | en-3-one |
| 16 | H | 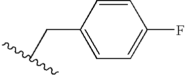 | Me | O | 323.2 | 14 | n.d. | n.d. | n.d. | 9a-(4-fluorobenzyl)-7-hy- droxy-4-methyl-1,2,9,9a-tetra- hydro-3H-fluor- en-3-one |
| 17 | H | 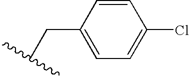 | Me | O | 340.4 | 21.7 | 6830 | n.d. | n.d. | 9a-(4-chlorobenzyl)-7-hy- droxy-4-methyl-1,2,9,9a-tetra- hydro-3%{H}-fluor- en-3-one |
| 18 | H | 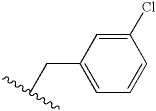 | Me | O | 339.0 | 15 | n.d. | n.d. | n.d. | 9a-(3-chlorobenzyl)-7-hy- droxy-4-methyl-1,2,9,9a-tetra- hydro-3%{H}-fluor- en-3-one |
| 19 | H | 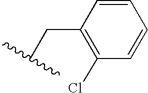 | Me | O | 339.2 | 66.6 | n.d. | n.d. | n.d. | 9a-(2-chlorobenzyl)-7-hy- droxy-4-methyl-1,2,9,9a-tetra- hydro-3%{H}-fluor- en-3-one |
| 20 | H | 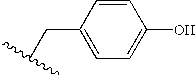 | Me | O | 321.2 | 85 | n.d. | n.d. | n.d. | 7-hydroxy-9a-(4-hy- droxybenzyl)-4-meth- yl-1,2,9,9a-tetra- hydro-3%{H}-fluor- en-3-one |
| 21 | H | 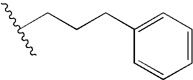 | Me | O | 332.8 | 11.4 | n.d. | n.d. | n.d. | 7-hydroxy-4-meth- yl-9a-(3-phe- nylpropyl)-1,2,9,9a-tetra- hydro-3%{H}-fluor- en-3-one |
| 22 | H | benzyl | Me | N—OMe | n.d. | 60 | n.d. | n.d. | n.d. | (3%{E})-9a-benzyl-7-hy- |
| | | | | | | | | | | droxy-4-methyl-1,2,9,9a-tetra- |
| | | | | | | | | | | hydro-3%{H}-fluor- |
| | | | | | | | | | | en-3-one |
| | | | | | | | | | | %{O}-methyloxime |
| 23 | H | benzyl | Me | N—OH | n.d. | 199 | n.d. | n.d. | n.d. | 9a-benzyl-8-bromo-7-hy- |
| | | | | | | | | | | droxy-4-methyl-1,2,9,9a-tetra- |
| | | | | | | | | | | hydro-3%{H}-fluor- |
| | | | | | | | | | | en-3-one |
| | | | | | | | | | | oxime |
| 24 | Br | benzyl | Me | O | 383.0/384.4 | 436 | NA at | n.d. | n.d. | 9a-benzyl-6,8-di- |
| | | | | | | | 180000 | | | bromo-7-hydroxy-4-meth- |
| | | | | | | | nM | | | yl-1,2,9,9a-tetra- |
| | | | | | | | | | | hydro-3%{H}-fluor- |
| | | | | | | | | | | en-3-one |

TABLE 2

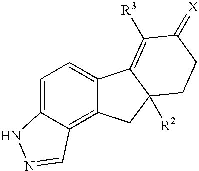

| | | | | | GST-ERα | GST-ERα | | | |
|---|---|---|---|---|---|---|---|---|---|
| | | | | | MG-LBD | wt-LBD | hERα | hERβ | |
| CMP | R2 | R3 | X | MS | IC50 [nM] | IC50 [nM] | IC50 [nM] | IC50 [nM] | Name |
| 1 | benzyl | H | O | 315.0 | 3900 | >10000 | >10000 | >10000 | 9a-benzyl-8,9,9a,10-tetra- |
| | | | | | | | | | hydroindeno[2,1-e]in- |
| | | | | | | | | | dazol-7(3H)-one |
| 5 | benzyl | Me | O | 329.0 | 155 | >10000 | >10000 | 1247 | 6-methyl-9a-ben- |
| | | | | | | | | | zyl-8,9,9a,10-tetra- |
| | | | | | | | | | hydroindeno[2,1-e]in- |
| | | | | | | | | | dazol-7(3H)-one |
| 9 | R2: benzyl R3: 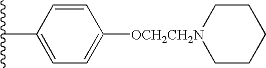 | | O | 518.2 | 107 | 1126 | 622 | 125 | 9a-benzyl-6-{4-[2-(1-pipe- ridinyl)eth- oxy]phenyl}-8,9,9a,10-tetra- hydroindeno[2,1-e]in- dazol-7(3H)-one hydrotrifluoroacetae salt |
| 25 | 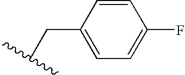 | Me | O | 347.0 | 82 | n.d. | >10000 | 997 | 6-methyl-9a-(4-fluoro- benzyl)-8,9,9a,10-tetra- hydroindeno[2,1-e]in- dazol-7(3H)-one |
| 26 | 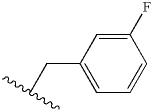 | Me | O | 347.0 | 444 | n.d. | >10000 | 1068 | 6-methyl-9a-(3-fluoro- benzyl)-8,9,9a,10-tetra- hydroindeno[2,1-e]in- dazol-7(3H)-one |
| 27 | 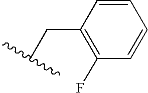 | Me | O | 347.0 | 135 | n.d. | 4298 | 529 | 6-methyl-9a-(2-fluoro- benzyl)-8,9,9a,10-tetra- hydroindeno[2,1-e]in- dazol-7(3H)-one |
| 28 | 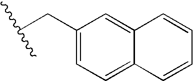 | Me | O | n.d. | 5500 | n.d. | n.d. | n.d. | 6-methyl-9a-(2-naph- thylmethyl)-8,9,9a,10-tetra- hydroindeno[2,1-e]in- dazol-7(3H)-one |
| 29 | 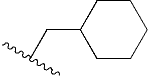 | Me | O | 335.2 | 146 | n.d. | n.d. | n.d. | 6-methyl-9a-cyclo- hexylmethyl-8,9,9a,10-tetra- hydroindeno[2,1-e]in- dazol-7(3H)-one |

TABLE 3

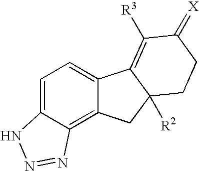

| | | | | | GST-ERα | GST-ERα | | | |
|---|---|---|---|---|---|---|---|---|---|
| | | | | | MG-LBD | wt-LBD | hERα | hERβ | |
| CMP | R2 | R3 | X | MS | IC50 [nM] | IC50 [nM] | IC50 [nM] | IC50 [nM] | Name |
| 30 | benzyl | Me | O | 330.0 | 1163 | n.d. | >10000 | 4186 | 9a-benzyl-6-methyl-8,9,9a,10-tetra- |
| | | | | | | | | | hydrofluoreno[1,2-d][1,2,3]tri- |
| | | | | | | | | | azol-7(3H)-one |
| 31 | 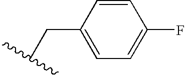 | Me | O | 348.0 | 340 | n.d. | >10000 | >10000 | 9a-(4-fluorobenzyl)-6-meth- yl-8,9,9a,10-tetra- hydrofluoreno[1,2-d][1,2,3]tri- azol-7(3H)-one |
| 32 | 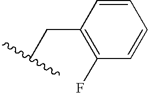 | Me | O | 348.2 | 2450 | n.d. | n.d. | n.d. | 9a-(2-fluorobenzyl)-6-meth- yl-8,9,9a,10-tetra- hydrofluoreno[1,2-d][1,2,3]tri- azol-7(3H)-one |
| 33 | 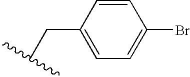 | Me | O | 408.0/ 410.0 | 880 | n.d. | n.d. | n.d. | 9a-(4-bromobenzyl)-6-meth- yl-8,9,9a,10-tetra- hydrofluoreno[1,2-d][1,2,3]tri- azol-7(3H)-one |
| 34 | 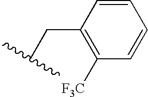 | Me | O | 398.0 | 4100 | n.d. | >10000 | >10000 | 9a-[2-(tri- fluoromethyl)benzyl]-6-meth- yl-8,9,9a,10-tetra- hydrofluoreno[1,2-d][1,2,3]tri- azol-7(3H)-one |
| 35 | 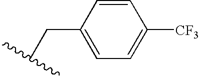 | Me | O | 398.0 | 6900 | n.d. | n.d. | n.d. | 9a-[4-(tri- fluoromethyl)benzyl]-6-meth- yl-8,9,9a,10-tetra- hydrofluoreno[1,2-d][1,2,3]tri- azol-7(3H)-one |
| 36 | 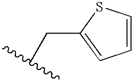 | Me | O | 336.0 | 4000 | n.d. | n.d. | n.d. | 9a-(thien-2-ylmethyl)-6-meth- yl-8,9,9a,10-tetra- hydrofluoreno[1,2-d][1,2,3]tri- azol-7(3H)-one |
| 37 | 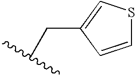 | Me | O | 336.0 | 2300 | n.d. | n.d. | n.d. | 9a-(thien-2-ylmethyl)-6-meth- yl-8,9,9a,10-tetra- hydrofluoreno[1,2-d][1,2,3]tri- azol-7(3H)-one |
| 38 | 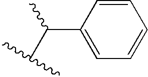 | Me | O | 344.0 | 3300 | n.d. | n.d. | n.d. | 9a-(1-phenylethyl)-6-meth- yl-8,9,9a,10-tetra- hydrofluoreno[1,2-d][1,2,3]tri- azol-7(3H)-one |

### Example 7

### Design and Construction of an ER-LBD Mutant Library

Because it had previously been shown to work in the context of chimeric constructs, the isolated ERA-LBD represented an attractive candidate to develop the veneered LBD needed for a truly orthogonal transcriptional switch. Different from its natural isoforms, the veneered LBD should be unable to bind the ligands of ER, such as estradiol, but able to bind an inactive analogue of the ligands. Although it is structurally related to the ligands, the inactive analogue is unable to bind the natural estrogen receptor.

We examined possible candidates for the inactive analogue according to the following criteria. First, the candidates should be inactive compounds (against both hERα and hERβ) within an otherwise generally active series. Second, the general structures of the candidates should require only a limited modification around the ligand-binding pocket of hERα LBD. Third, the candidates preferably show a generally acceptable pharmacokinetic profile.

According to these criteria it was decided to synthesize CMP1 (FIG. 1A), a compound with a large benzyl substitution at the 9a position. Choice of the 9a position was motivated by the observation that modification at this position in a series of hERβ-selective tetrahydrofluorenone ligands developed from an in-house program generally caused a strong reduction in binding affinity. Furthermore, replacement of the phenolic hydroxy group by a pyrazole heterocycle was known to improve the pharmacological properties of the active series. The newly synthesized CMP1 indeed displayed a very poor binding affinity for both hERα and hERβ (IC₅₀ values>10⁴ nM, respectively, FIG. 1A) and represented therefore a promising starting point around which a receptor mutagenesis strategy could be designed.

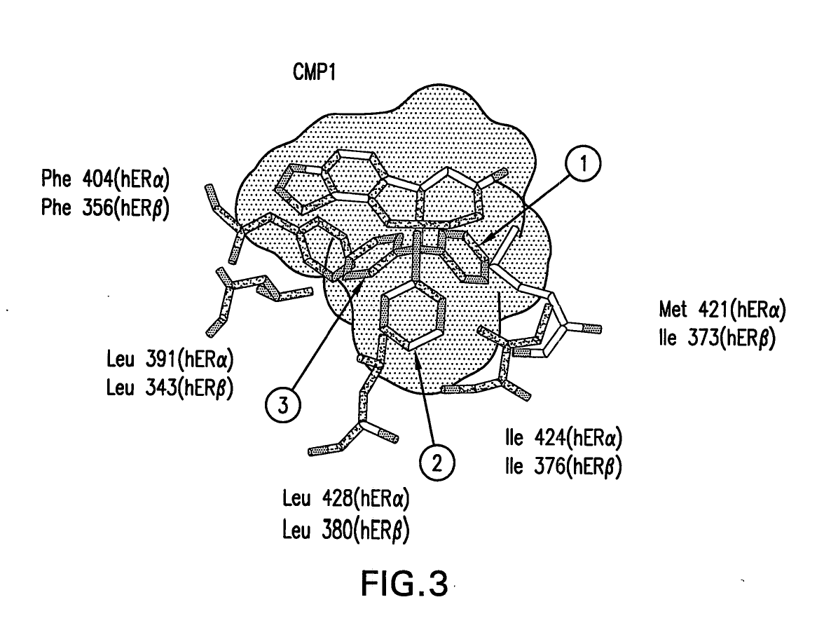

According to the X ray crystal structures of agonist-bound hERα and hERβ LBDs, CMP1 was modeled into the hERα and hERβ binding pockets assuming three distinct major conformations for the 9a benzyl substituent (R², table 2) predicted by energy calculations (FIG. 3). Five residues within the ligand-binding pocket were identified as the most likely candidate residues interfering with binding of CMP1:L391/L343, F404/F356, M421/I373, I424/I376, I428/L380 of hERα or hERβ, respectively. A mutant library including these five positions should cover nearly 300° of conformational space of the 9a benzyl rotamer (R², table 2), including all three major conformations of the benzyl substituent.

Because the interference with binding of CMP1 was expected to arise from steric clashes between the side-chains and the 9a benzyl moiety (R², table 2), substitutions into smaller or, more generally, different residues should remove this hinderance. Due to the generally hydrophobic character of both the binding pocket and the benzyl substituent, we decided to use only Gly, Ala, Cys, Val, Be, Leu, Met, Phe, Tyr or Trp as possible substitutions at the five positions.

The analysis further showed that differences between hERα and hERβ affecting binding of CMP1 should be restricted to only two residues, L384/M336 and M421/I373 of hERα and hERβ, respectively. Since the pyrazole compound series showed preferential binding to hERβ (FIG. 1A), we decided to generate the library in the context of a L(384)M mutated hERα. The second residue, M421, corresponded to one of the positions mutated in the library and inclusion of Ile as a possible substitution for M421 therefore implicitly generated a library containing also the wt hERβ ligand-binding pocket.

Choice of hERα over hERβ was motivated by two factors. First, the biology of hERα was much better known. Second, related successful experiments using the hERα ligand-binding domain (LBD) had already been described in the literature. In vitro experiments using GST-fusion constructs with single or combined L(384)M and M(421)I variants of the hERα LBD reproduced the binding data observed for the full-length wt hERβ (FIG. 1A).

### Example 8

### Validation of the Library Complexity in the Context of a Yeast Selection System

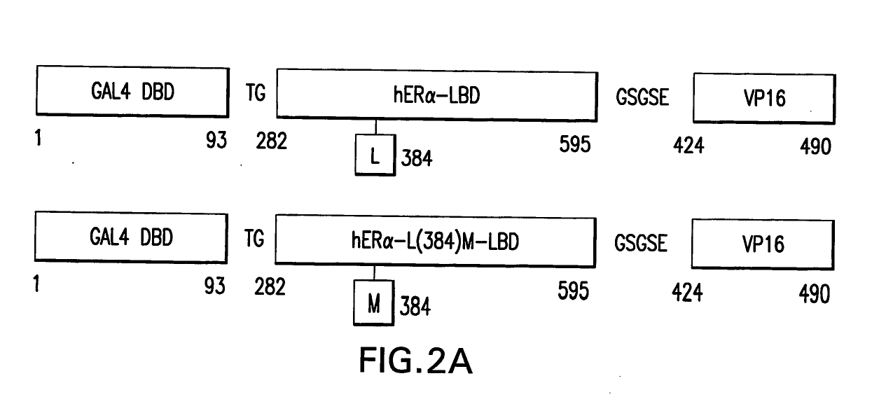

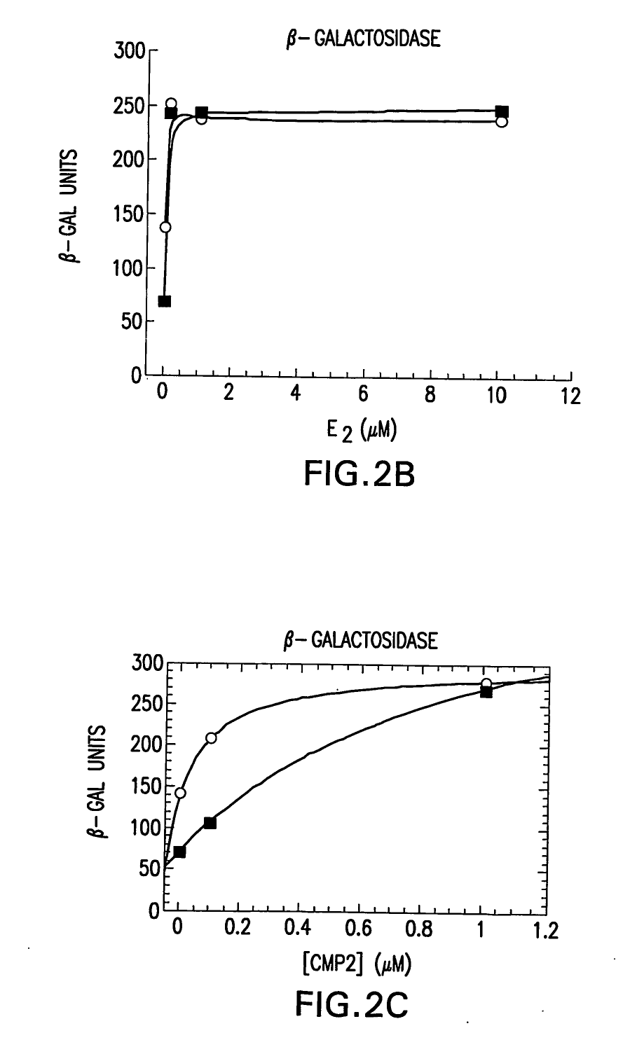

In order to genetically select for CMP1-responsive variants, the L384M mutated hERα LBD was fused to the GAL4 DNA-binding domain (DBD) and the VP16 transactivation domain (FIG. 2A). The responsiveness of this chimera to the pyrazole series of compounds was determined through a titration curve against estradiol (E2) (FIG. 2B) or the active compound CMP2 (FIG. 2C). As before, the L(384)M substitution was both necessary and sufficient to modulate the affinity for the chimeric transcription factor. In addition, it demonstrated that the pyrazole series of compounds was able to penetrate the yeast membrane, a property that is frequently absent thus impeding the use of the yeast system as a genetic selector.

Generation of the library of hERα-LBD variants took advantage of the inherent yeast recombination system. Two oligonucleotides degenerate for 10 different amino acids at each of the five library positions were synthesized using a di-nucleotide assembly strategy, including sufficiently long (>30 bp) constant flanking regions to direct the homologous recombination.

To decrease the likelihood that clones with single- or double-mutations would be lost during the synthesis, construction or selection of the library, the concentration of wt codons was raised to 15% for each of the mutated positions. As a consequence the relative abundance of 1- or 2-residue variants was significantly raised (by about 8- and 4-fold, respectively) compared to only a minor reduction in the relative abundance of 5-residue variants (factor of about 0.8). This strategy was also in line with our expectation that predominantly 1- or 2-residue variants rather than more complex variants would be necessary to accommodate the CMP1 ligand, and, at the same time, be tolerated by the LBD structural framework.

Preparative amounts of the mutated LBD fragments were synthesised by PCR amplification of hER LBD template using the degenerated oligonucleotide mix. The mutated fragment collection was then included in a scaled-up cotransformation experiment together with a linearised recipient L(384)M hER LBD vector. Approximately 10⁶ colonies corresponding to a 10-fold library redundancy were plated, and 12 randomly chosen clones revealed the expected library complexity (data not shown).

In order to probe the library for selection capacity, −His/−Trp growth-selective plates were treated with increasing amounts of the hERβ-selective CMP3. Sequencing of 9 randomly picked clones indicated that, different from the non-selected clones, most of the CMP3-selected clones had either zero or few amino acid substitutions in all five mutagenized positions, being L391 and M421 residues completely conserved (data not shown). It appears therefore that a very restricted number of mutations in the hER binding pocket is tolerated to maintain a productive interaction with a high affinity ligand of the wt receptor. No clones containing the M(421)I substitution were selected, consistent with the observation that no further increase in the binding affinity for CMP3 was observed by adding this second substitution to the L(384)M mutated protein (FIG. 1A).

### Example 9

### Genetic Library Screening and Analysis of Selected ER-LBD Mutated Variants

Having confirmed the expected composition and responsiveness of the library, we repeated the growth selection in the presence of the inactive analogue CMP1 at a concentration of 1 μM. 1600 independent clones were subsequently screened against 1 μM CMP1 or DMSO, as a control, on −Trp/X-gal-containing plates. 54 independent clones were judged positive for β-Galactosidase trans-activation in this experiment and 28 amongst them retained positively also in the presence of 0.1 μM CMP1. A parallel experiment was carried out using the same strategy but performing the initial growth selection and first round of white/blue screening in the presence of a 10-fold higher compound concentration (10 μM). 31 clones retained positively when challenged with 0.1 μM CMP1 and were therefore selected for further characterization together with the 28 positive clones of the former experiment.

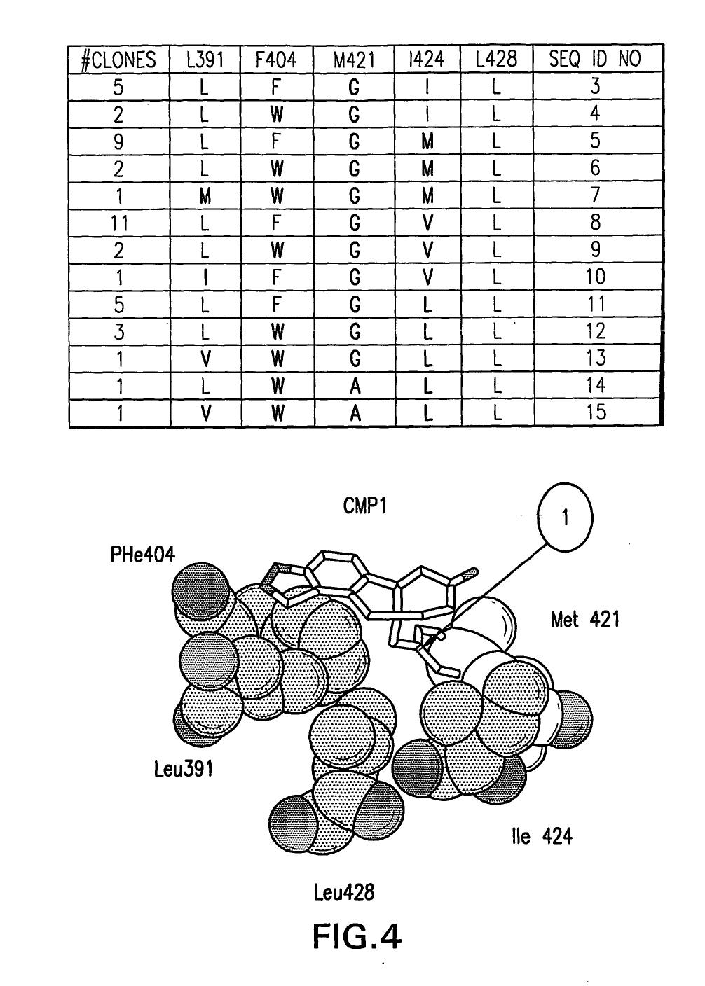

DNA sequence analysis revealed a consensus sequence of the selected mutant variants (FIG. 4 and SEQ ID NO: 3-15). The most prominent feature was the mutation of M421 into a residue containing a smaller side chain (mostly G and few A) that occurred in 86% of the selected clones. Importantly, this mutation was never observed in clones selected using the active pyrazole compound CMP3 (data not shown), thus excluding a bias in the screening procedure. An isolated M421G mutation was found with a relatively low frequency and a second mutation of I424 to either M, V or L was present in most of the clones. In addition, a third mutation of F404 to W was present in 28% of the mutated clones, whereas positions L391 and L428 were generally conserved. The consensus sequence present in most selected variants is consistent with a model in which the benzyl substituent of CMP1 is directed towards the positions originally occupied by M421 and I424 (FIG. 4).

Western blot analysis of yeast protein extracts from all representative clones shown in FIG. 4 using a monoclonal antibody directed against VP16 AD did not show significant differences in the expression levels of the corresponding chimeric proteins (not shown).

### Example 10

### Novel Tetrahydrofluorenone Compounds Interact Selectively with ER-LBD Variants Mutated in M421

Representative selected arrays of mutations shown in FIG. 4 were introduced in the pGEX-hERα-L(384)M-LBD prokaryotic expression construct and the corresponding GST-fusion protein variants were expressed in E. coli. Suitable aliquots of crude bacterial extracts containing comparable amounts of all different protein variants were tested for direct binding in vitro to increasing concentrations of 3[H]-estradiol. All variants bound the natural ligand E2 with a reduced affinity compared to the hERα-L(384)M-LBD protein (wt in all 5 mutagenized positions) (data not shown). The single M(421)G-selected mutant bound estradiol with the highest affinity with a k_{d} value approximately 9-fold higher than wt (46 nM and 5 nM, respectively) (FIG. 1A). The k_{d} value for the hormone was nevertheless sufficiently low to measure the affinity of this polypeptide for a series of novel tetrahydrofluorenone compounds including CMP1, CMP4, CMP5 (FIG. 1A) and all those listed in tables 1a, 1b, 2, and 3 in ³[H]E₂-displacement assays. A glycine in position 421 was confirmed to confer selectivity of binding to the majority of the compounds tested (FIG. 1A and tables 1b, 2, 3). The affinity for tetrahydrofluorenones containing a phenol group was significantly higher than for those containing a pyrazole substituent. Furthermore, the binding affinity was higher for compounds with a methyl group in position 4 than for those containing a hydrogen atom in the corresponding position.

### Example 11

### ER-LBD Variants Mutated in M421 Show Fluorenone-Dependent Transcriptional Activity

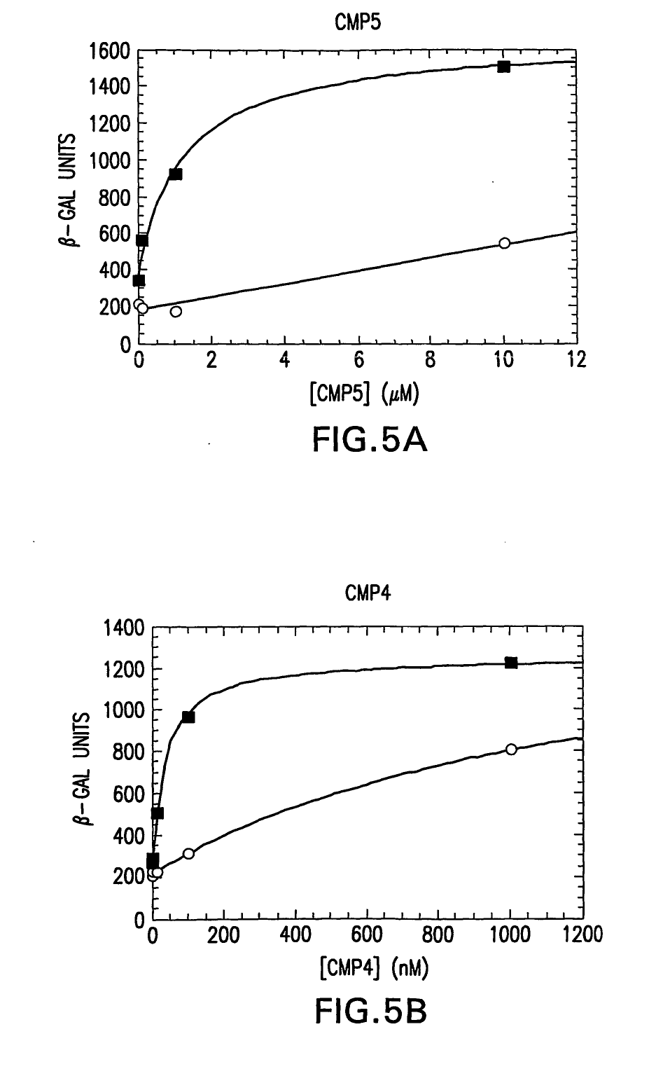

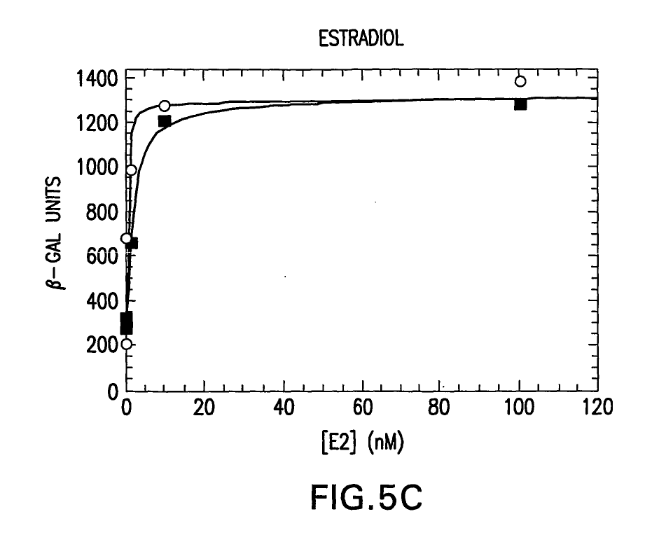

A series of representative selected mutants was transferred in the yeast strain Y187, in which the integrated lacZ reporter gene is under the control of the intact GAL1 promoter and is therefore more tightly regulated and more efficiently expressed than in the yeast strain CG-1945 (the library-containing strain). GAL4/VP16 chimeras containing ER LBD L(384)M with or without the additional selected mutation M(421)G were tested in ligand-dependent β-Galactosidase trans-activation experiments in the presence of E₂, CMP4 or CMP5 compounds (FIG. 1A). Representative experiments are shown in FIGS. 5A-5C. Our results confirmed that the mutant containing a glycine in position 421 was selectively activated by both CMP4 and CMP5 cognate ligands with EC₅₀ values of 36 nM and 217 nM, respectively, comparable with the IC₅₀ values measured in vitro (FIG. 1A).

We also reversed the original mutation L(384)M in the context of the M(421)G selected mutant, thus generating a single-substituted ERα LBD M(421)G chimeric protein. The transcriptional activity induced in yeast by the pyrazole compound CMP5 was strongly impaired in the absence of the L(384)M mutation (data not shown), indicating that the combination of the two amino acid substitutions was necessary to define the ligand specificity of the selected mutant.

For what concerns the response to estradiol, the EC₅₀ value measured with the M(421)G mutant (1.5 nM) was approximately eight-fold higher than that associated with the parental clone (0.2 μM), a difference of the same order of magnitude of that measured in vitro (see FIG. 1A).

### Example 12

### ER-LBD Variants Bearing D(351)A/H524V Mutations Respond Poorly to Estradiol and Show Low Ligand-Independent Activity

At this point we had accomplished the first step obtaining ER-LBD variants with the desired shift in specificity towards the inactive analogues. To make the system practically useful, two additional essential properties had to be introduced. First, the L(384)M/M(421)G (MG)-LBD variant should be orthogonal against the natural ligand estradiol. Second, the ligand-independent transcriptional activity should be reduced to background levels.

We targeted amino acid residues which make contacts with the D-ring of estradiol in the crystal structure of the hER LBD-hormone complex. Since tetaahydrofluorenone compounds formally lack a structural equivalent of the hormone D-ring, we reasoned that alterations in the D-ring-interacting region of the LBD should not severely compromise binding to these ligands. His₅₂₄, hydrogen-bonded to the 17-hydroxyl of estradiol was therefore mutated to Val in the context of MG-LBD variant. Furthermore, Gly521, which makes hydrophobic van der Waals contacts with the D-ring of estradiol, was also substituted with Val, Leu, Met, or Arg. The corresponding GAL4/VP16 chimeric proteins harboring these new mutations were challenged in ligand-dependent β-Galactosidase transcription assays in the presence of either estradiol or ligand CMP4.

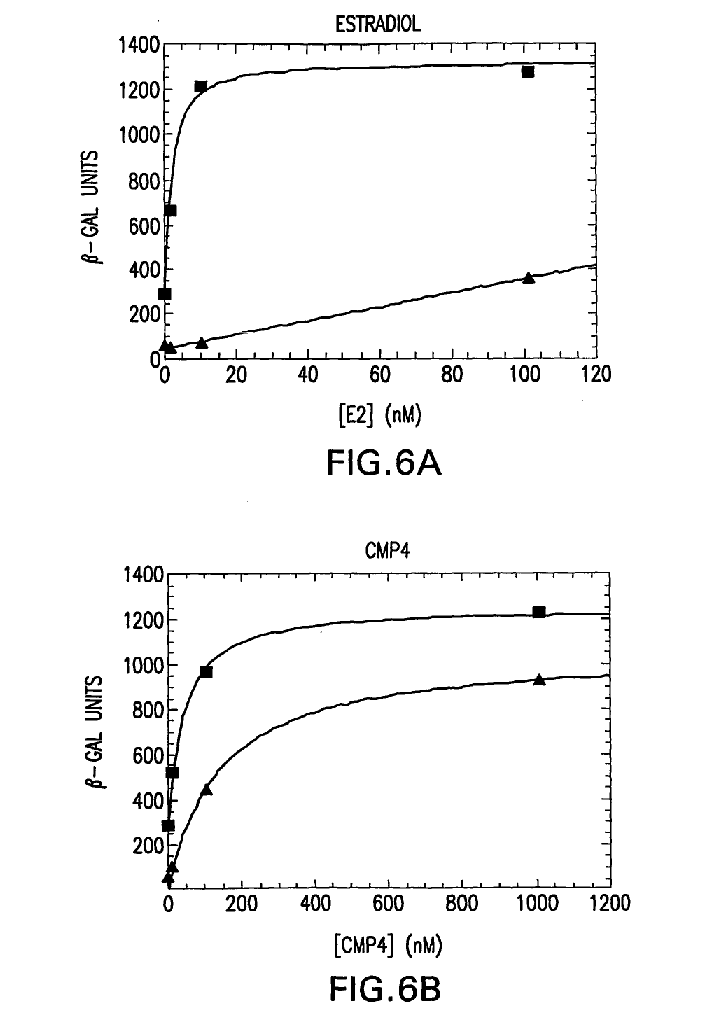

Although all substitutions of Gly₅₂₁ resulted in a significant decrease of both basal and estradiol-induced trans-activation levels in yeast, they also caused a dramatic loss of response to the fluorenone compound CMP4 (data not shown). In contrast, H524V displayed a strongly reduced affinity for estradiol of about 200-fold (FIG. 6A) and a concomitant reduction in affinity of only 3-fold for ligand CMP4 (FIG. 6B).

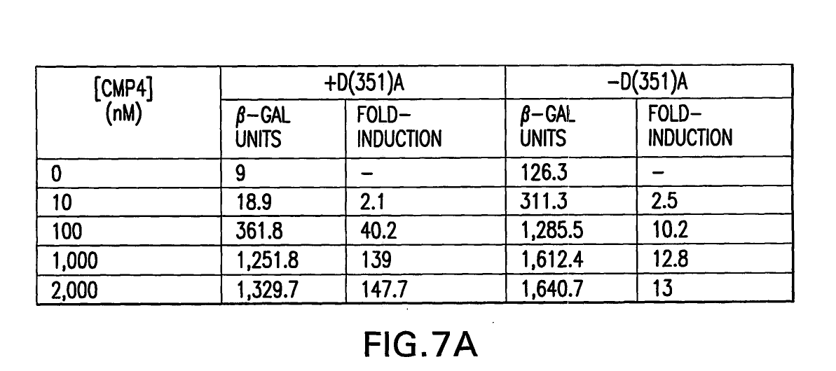

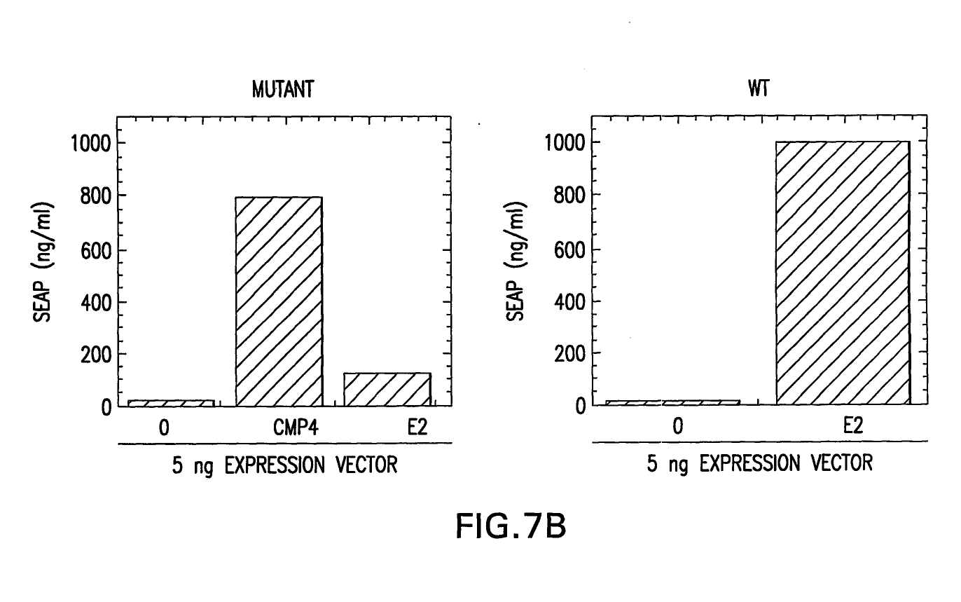

A third and important step to devise a transcription factor aimed for the regulation of trans-gene expression in vivo was to minimize its background activity in the absence of added ligand. The MG-LBD “lead” selected variant containing the additional H(524)V mutation described above still retained relative high constitutive activity levels (FIG. 7A).

Published data indicated that amino acid substitutions which abolish the negative charge of Asp₃₅₁ in the context of full-length hERα determine a significant decrease of its basal activity without interfering with any ligand-dependent response. Therefore, a D(351)A mutation was inserted in the MG-LBD “lead” selected variant containing the additional H(524)V mutation. The presence of the D(351)A mutation determined a 14-fold reduction of ligand-independent activity with a concomitant significant increase of the maximal fold-induction by the cognate ligand CMP4 (FIG. 7A).

Having fixed an array of mutations within hERα-LBD, [D(351)A, L(384)M, M(421)G, H(524)V] which changed the specificity of activation in response to ligands in yeast, we wished to verify that this altered specificity was maintained in mammalian cells. Therefore, we transferred both the mutated and wt GAL4/hERα-LBD/VP16 sequences in a mammalian expression vector and evaluated their ligand-dependent transcriptional activity by co-transfection in HeLa cells with a GAL4-responsive reporter plasmid expressing SEAP under the control of five GAL4 UAS repeats (5GAL4UAS-pSEAP). The ability of the mutant chimeric transcription factor to mediate reporter gene activation in response to the cognate ligand CMP4 and estradiol was compared with the response of the chimera containing the wt hERα-LBD.

The result shown in FIG. 7B demonstrates that the constitutive activity of the mutant was as low as that of wt. Maximal activity levels shown by the mutant in response to saturating concentrations of CMP4 were also comparable with those obtained with wt in response to saturating concentrations of estradiol (60-90 fold induction). Furthermore, the maximal response of the mutant to saturating concentrations of estradiol was significantly impaired (10% of that shown by wt).

### Example 13

### ER-LBD Variants Bearing a G521R Mutation Respond Poorly to Estradiol, Show Low Ligand-Independent Activity and can be Induced by Antagonistic Compounds

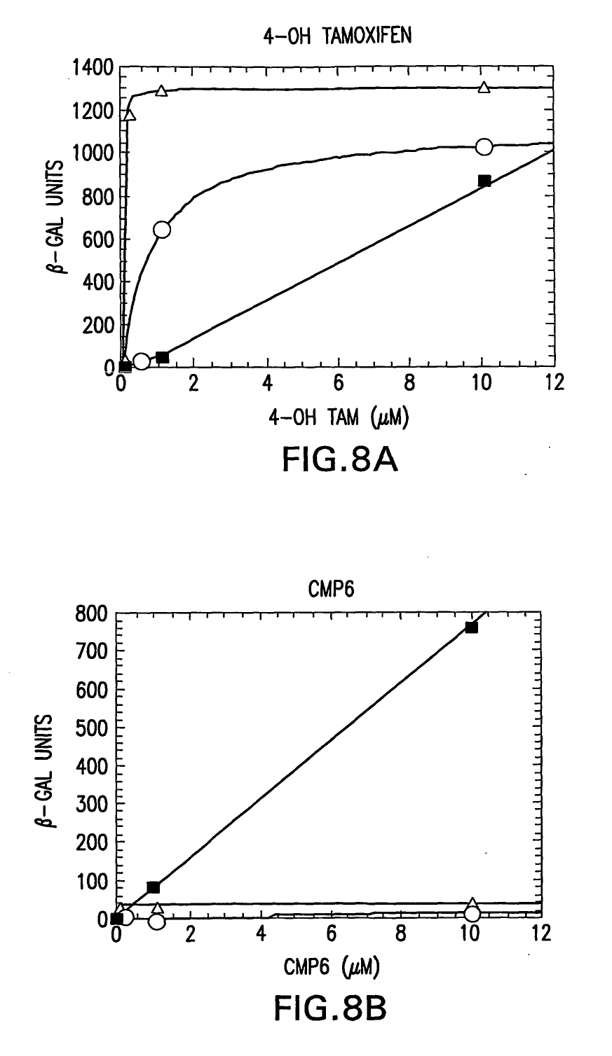

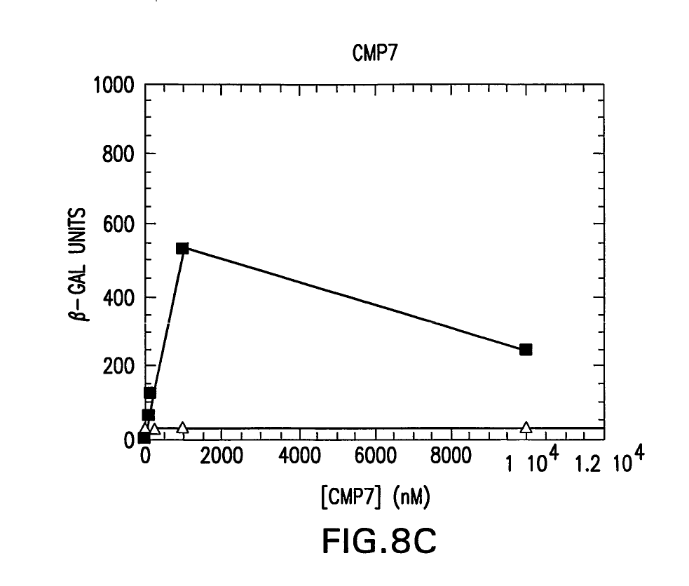

In addition to the H524V variant, we followed a different strategy taking advantage of the well-known biological properties of the ER-LBD G(521)R substitution. Estrogen receptors bearing this mutation in their LBD exhibit both the desired properties: a low basal transcriptional activity and strongly reduced affinity for the estradiol ligand. Since our experiments showed the incompatibility of the agonistic series of fluorenone compounds with the G(521)R mutation, possible modifications of the ligand were explored. The rationale behind this reasoning was the notion that the G(521)R-substituted MG-LBD variant, as described for the wt ER G(521)R, was still proficient in the response to 4 hydroxytamoxifen (4-OH Tam), an ER antagonist (FIG. 8A). More generally, ligands having an “antagonistic tail” apparently can compensate for loss of affinity introduced by G(521)R in the agonist binding pocket. A new series of compounds was therefore generated by substituting the methyl group at the R³ position with more bulky side-chains mimicking an “antagonistic tail” (FIG. 1B and table 1a). Both pyrazole (CMP9, FIG. 1B. See also table 2) and phenol derivatives (FIG. 1B and table 1a) containing different R³ side-chains were challenged in competitive radiometric in vitro assays for binding to the MG-LBD selected mutant and to ERα wt-LBD polypeptides. All compounds showed a significant selectivity for the mutant relative to the wt LBD and to both α and β full-length ER, albeit to different extent. Selectivity varied between 50 and 150 fold when the mutant and wt ERα LBD were compared, the differences being somewhat less pronounced on the full-length receptors. Although the presence of an antagonist side-chain apparently did not influence the binding affinity for the mutant when measured in vitro, no significant response to most of these compounds was detected in yeast transcriptional assays up to 10 μM concentrations (not shown). This suggests that the uptake by yeast cells of compounds containing bulky, positive-charged side-chains is strongly impaired, as already observed for many known anti-estrogens.

An exception was compound CMP6 containing a phenol substituent in R³ which was able to significantly induce in a concentration-dependent fashion the transcriptional activity of the L(384)M, M(421)G, G(521)R variant in yeast cells (FIG. 8B). On the contrary, only a very weak response was observed for a variant containing only the L(384)M and G(521)R mutations and the compound was completely ineffective on the wt ERα LBD chimera up to 10 μM concentration. Similar results were obtained for CMP7 (FIG. 8C), demonstrating that a butyl substituent in R³ is sufficient to confer an “antagonistic” nature to the ligand. More importantly, these data indicate that the specificity conferred by the ER-LBD M(421)G substitution for the phenyl group in the 9a position (R², table 1a) of the ligand was preserved. As expected, the relative response levels were reversed when 4-OH Tam was tested. The L(384)M, M(421)G, G(521)R variant showed indeed a significantly lower affinity for this antagonist compared with both the L(384)M and G(521)R and the wt ERα LBD chimera.

### Example 14

### Regulation of Gene Expression by Compounds of the Antagonistic Series

The combination of the L384M/M421G/G521R ER-LBD variant with an antagonistic ligand thus exhibited the desired properties in order to be applicable in gene therapy applications. To test the system in mammalian cells, we therefore modified a ligand-dependent transcription regulator, called HEA-1, that we have recently generated.

In HEA-1 three elements are fused together: the DNA-binding domain (DBD) of human HNF-1α (aa 1-282), a G(521)R mutant of the LBD of the human ERα (aa 303-595), and a portion of the activation domain of the human p65 protein (aa 285-551). HEA-1 promotes transcription of transgenes downstream a multiple HNF-1 binding site in a stringent 4-OH Tam dependent manner with up to hundred-fold drug-dependent transgene induction in cell culture (Roscilli et al., 2002 Mol Ther. 5:653-663). Moreover, HEA-1 enables tight regulation of gene expression in vivo by treating mice with Tamoxifen (TAM), which is predominantly metabolized to 4-OH Tam in vivo (REF) (Rosciffi et al., 2002 Mol Ther. 5:653-663).

The intrinsic antagonistic activities of TAM and its metabolite 4-OH Tam are however responsible for a number of side effects (such as increased incidence of uterine cancer) that become more pronounced during prolonged treatments and therefore might preclude the use of this system for long-term gene therapy applications. The use of inducer molecules with a very low affinity for endogenous ER-alpha and ER-beta should thus widen the potential application range of HEA-1-based transcription regulatory systems.

We therefore generated HEA-2 (SEQ ID NO: 43), which is a triple ER-LBD mutant L(384)M/M(421)G/G(521)R in the context of HEA-1, and tested its responsiveness to antagonist-type compounds. The amino acid sequence of HEA-2 is listed in SEQ ID NO 43. The corresponding cDNA was cloned into an eukaryotic expression vector and co-transfected in HeLa cells along with the reporter plasmid 7×H1/CRP/SEAP (Roscilli et al., 2002 Mol Ther. 5:653-663). Cells were treated with increasing concentrations of four antagonistic compounds and SEAP levels were measured in the culture medium after 24 hour treatment.

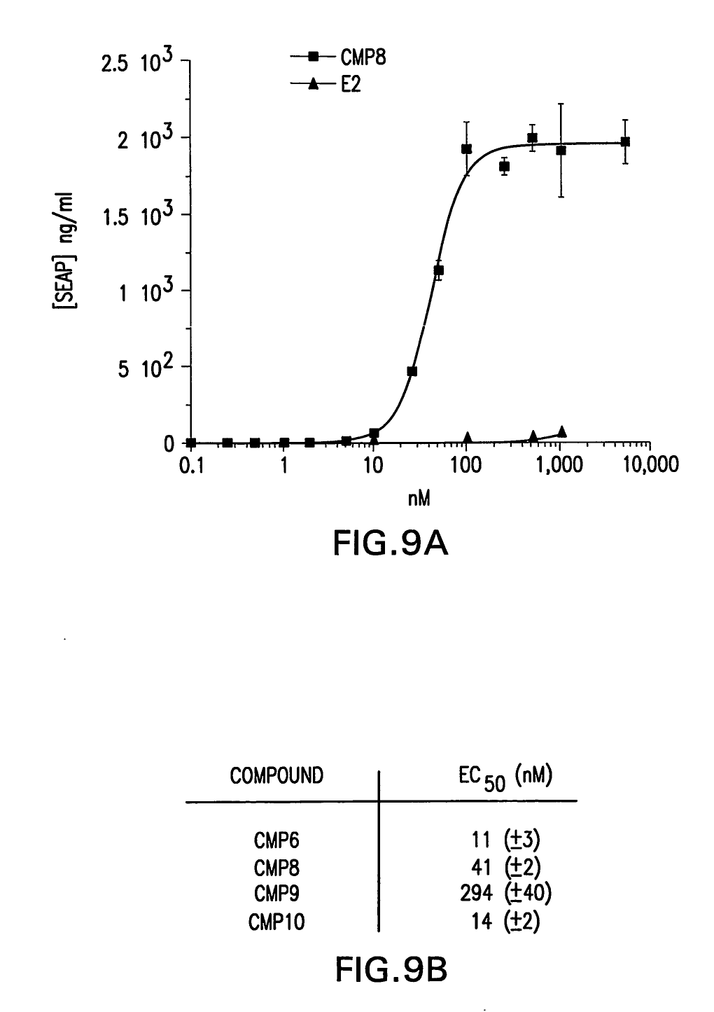

The results showed that the chimera was stimulated by all four compounds in a dose-dependent manner. A representative dose-response curve with CMP8 compound is shown in FIG. 9A. In the absence of treatment, a basal SEAP level of 0.8±0.2 ng/ml was measured, similar to that measured in the culture medium of cells transfected only with the reporter plasmid (not shown). This indicated the absence of a significant basal activity in this experimental setting. About 1900 ng/ml of SEAP were measured at the highest concentration of CMP8, which corresponds to about 2000-fold increase as compared to the uninduced condition. Similar fold inductions were consistently observed in duplicate experiments and with all the four compounds (not shown).

FIG. 9A also demonstrates that the chimera is activated by E₂ only at the highest concentrations of the hormone (17-fold and 50-fold induction at 500 nM and 1 μM E₂). Consistent with the in vitro binding analysis (FIG. 1B), CMP9 displayed the lowest activity with an EC₅₀ of approximately 300 nM, while the EC₅₀ of the other compounds ranged from 11 to 40 nM (FIG. 9B).

Other embodiments are within the following claims. While several embodiments have been shown and described, various modifications may be made without departing from the spirit and scope of the present invention.

What is claimed:

1. A polypeptide comprising,
SAGDMRAANL WPSPLMIKRS KKNSLALSLT ADQMVSALLD
AEPPILYSEY DPTRPFSEAS MMGLLTNLAX₁ RELVHMINWA
KRVPGFVDLT LHDQVHLLEC AWMEILMIGX₂ VWRSMEHPGK
LLX₃APNLLLD RNQGKCVEGX₄ VEX₅FDMX₆LAT SSRFRMMNLQ
GEEFVCLKSI ILLNSGVYTF LSSTLKSLEE KDHIHRVLDK
ITDTLIHLMA KAGLTLQQQH QRLAQLLLIL SHIRHMSNKX₇
MEX₈LYSMKCK NVVPLYDLLL EMLDAHRLHA PTSRGGASVE
ETDQSHLATA GSTSSHSLQK YYITGEAEGF PATV
wherein X₁=D or A; X₂-6 are each independently G, A, C, V, I, L; M, F, Y, or W; X₇ G or R;
and X₈=H or V.

2. The polypeptide of claim 1 wherein X₂=L, M, or V; X₃=F or W; X₄=M, G or A; X₅=I, M, V, or L; X₆=L.

3. The polypeptide of claim 2 wherein X₁=D; X₇=G; and X₈=H.

4. The polypeptide of claim 3 comprising the amino acid sequence of SEQ ID NO: 2.

5. The polypeptide of claim 3 wherein X₄=G or A.

6. The polypeptide of claim 5 comprising an amino acid sequence selected from the group consisting of SEQ ID NO: 3-15.

7. The polypeptide of claim 2 wherein X₁=A, X₄=G or A; X₇=G, and X₈=V.

8. The polypeptide of claim 7 comprising an amino acid sequence selected from the group consisting of SEQ ID NO: 16-28.

9. The polypeptide of claim 2 wherein X₁=D, X₄=G or A; X₇=R, and X₈=H.

10. The polypeptide of claim 9 comprising an amino acid sequence selected from the group consisting of SEQ ID NO: 29-41.

11. A polynucleotide encoding the polypeptide of claim 1.

12. A transcription factor comprising,
a DNA-binding domain,
a ligand-binding domain comprising the polypeptide of claim 1, and
a transcription regulatory domain.

13. The transcription factor of claim 12 wherein the ligand-binding domain comprises an amino acid sequence selected from the group consisting of SEQ ID NO: 16-41.

14. The transcription factor of claim 12 wherein the DNA-binding domain is GAL4 minimal DNA-binding domain.

15. The transcription factor of claim 12 wherein the DNA-binding domain is the DNA-binding domain of HNF-1.

16. The transcription factor of claim 12 wherein the transcription regulatory domain is VP16 minimal activation domain.

17. The transcription factor of claim 12 wherein the transcription regulatory domain is a portion of the activation domain of human p65.

18. The transcription factor of claim 12 comprising SEQ ID NO: 43.

19. A polynucleotide encoding the transcription factor of claim 12.

20. A host cell transformed with a composition comprising the polynucleotide of claim 18.

21. A compound that binds to and activates the transcription factor of claim 12.

22. The compound selected from the group consisting of CMP1 and CMP4-38.

23. An orthogonal gene switch for regulating the expression of a desired gene, the gene switch comprising, the transcription factor of claim 13; and a vector comprising the desired gene, and a regulatory region that is fused to the desired gene, wherein the transcription factor is capable of binding to the regulatory region.

24. The gene switch of claim 23 further comprising a compound that binds to the ligand-binding domain and activates the transcription factor.

25. The gene switch of claim 24 wherein the ligand-binding domain comprises an amino acid sequence selected from the group consisting of SEQ ID NO: 16-28, and the compound is selected from the group consisting of CMP1, CMP4, CMP5, and CMP11-38.

26. The gene switch of claim 23 wherein the ligand-binding domain comprises an amino acid sequence selected from the group consisting of SEQ ID NO: 29-41, and the compound is selected from the group consisting of CMP6-10.

27. A method of making an orthogonal gene switch, the method comprising:
selecting a ligand-binding domain (BD) from a nuclear hormone receptor,
selecting an inactive analogue of the hormone; constructing a library of transcription factors comprising veneered variants of the selected LBD, which are created by mutating amino acid residues that hinder the binding of the selected inactive analogue to various amino acid residues that might facilitate the binding; and screening the library with the selected inactive analogue to select the transcription factors that are activated by the inactive analogue.

28. The method of claim 27 further comprising introducing mutations into the veneered LBDs of the selected transcription factors to reduce their affinity to the hormone and the ligand-independent activity of the transcription factors.

29. The method of claim 28 further comprising making inactive analogues that are capable of activating the transcription factors carrying the mutations.

30. The method of claim 27 wherein the nuclear hormone receptor is selected from the group consisting of estrogen receptor (ER), androgen receptor (AR), glucocorticoid receptor (GR), mineralocorticoid receptor (MR), progesterone receptor (PR), vitamin D₃ receptor (VDR), thyroid hormone receptor (TR), and retinoic acid receptor (RAR).

31. The method of claim 29 wherein the nuclear hormone receptor is human estrogen receptor α (hERα).

32. The method of claim 30 wherein the ligand-binding domain comprising SEQ ID NO: 2.

33. The method of claim 27 wherein the inactive analogue is an inactive analogue of a nuclear hormone receptor-specific agonist or antagonist.

34. The method of claim 33 wherein the inactive analogue is an inactive analogue of hERα-specific agonist or antagonist.

35. The method of claim 33 wherein the inactive analogue is an inactive analogue of a human estrogen receptor β (hERβ)-specific agonist or antagonist.

36. The method of claim 35 wherein the inactive analogue is CMP1.

37. The method of claim 27 wherein the library is a yeast one hybrid system.

38. The method of claim 27 wherein the transcription factor further comprising a GAL4 minimal DNA-binding domain (DBD) and a VP16 minimal activation domain (AD).

39. The method of claim 32 wherein the library of transcription factors contains veneered LBDs with their amino acid residues 391, 404, 421, 424, and 428 independently selected from the group consisting of Gly, Ala, Cys, Val, Ile, Leu, Met, Phe, Tyr, and Trp.


SEQUENCE LISTING (62 sequences)

SEQ ID NO: 1 - protein, 314 aa, Human
SAGDMRAANLWPSPLMIKRSKKNSLALSLTADQMVSALLDAEPPILYSEYDPTRPFSEASMMGLLTNLADRELVHMINWAKRVPGFVDLTLHDQVHLLECAWLEILMIGLVWRSMEHPGKLLFAPNLLLDRNQGKCVEGMVEIFDMLLATSSRFRMMNLQGEEFVCLKSIILLNSGVYTFLSSTLKSLEEKDHIHRVLDKITDTLIHLMAKAGLTLQQQHQRLAQLLLILSHIRHMSNKGMEHLYSMKCKNVVPLYDLLLEMLDAHRLHAPTSRGGASVEETDQSHLATAGSTSSHSLQKYYITGEAEGFPATV

SEQ ID NO: 2 - protein, 314 aa, Artificial Sequence
  human sequence with a point mutation
SAGDMRAANLWPSPLMIKRSKKNSLALSLTADQMVSALLDAEPPILYSEYDPTRPFSEASMMGLLTNLADRELVHMINWAKRVPGFVDLTLHDQVHLLECAWMEILMIGLVWRSMEHPGKLLFAPNLLLDRNQGKCVEGMVEIFDMLLATSSRFRMMNLQGEEFVCLKSIILLNSGVYTFLSSTLKSLEEKDHIHRVLDKITDTLIHLMAKAGLTLQQQHQRLAQLLLILSHIRHMSNKGMEHLYSMKCKNVVPLYDLLLEMLDAHRLHAPTSRGGASVEETDQSHLATAGSTSSHSLQKYYITGEAEGFPATV

SEQ ID NO: 3 - protein, 314 aa, Artificial Sequence
  human sequence with point mutations
SAGDMRAANLWPSPLMIKRSKKNSLALSLTADQMVSALLDAEPPILYSEYDPTRPFSEASMMGLLTNLADRELVHMINWAKRVPGFVDLTLHDQVHLLECAWMEILMIGLVWRSMEHPGKLLFAPNLLLDRNQGKCVEGGVEIFDMLLATSSRFRMMNLQGEEFVCLKSIILLNSGVYTFLSSTLKSLEEKDHIHRVLDKITDTLIHLMAKAGLTLQQQHQRLAQLLLILSHIRHMSNKGMEHLYSMKCKNVVPLYDLLLEMLDAHRLHAPTSRGGASVEETDQSHLATAGSTSSHSLQKYYITGEAEGFPATV

SEQ ID NO: 4 - protein, 314 aa, Artificial Sequence
  human sequence with point mutations
SAGDMRAANLWPSPLMIKRSKKNSLALSLTADQMVSALLDAEPPILYSEYDPTRPFSEASMMGLLTNLADRELVHMINWAKRVPGFVDLTLHDQVHLLECAWMEILMIGLVWRSMEHPGKLLWAPNLLLDRNQGKCVEGGVEIFDMLLATSSRFRMMNLQGEEFVCLKSIILLNSGVYTFLSSTLKSLEEKDHIHRVLDKITDTLIHLMAKAGLTLQQQHQRLAQLLLILSHIRHMSNKGMEHLYSMKCKNVVPLYDLLLEMLDAHRLHAPTSRGGASVEETDQSHLATAGSTSSHSLQKYYITGEAEGFPATV

SEQ ID NO: 5 - protein, 314 aa, Artificial Sequence
  human sequence with point mutations
SAGDMRAANLWPSPLMIKRSKKNSLALSLTADQMVSALLDAEPPILYSEYDPTRPFSEASMMGLLTNLADRELVHMINWAKRVPGFVDLTLHDQVHLLECAWMEILMIGLVWRSMEHPGKLLFAPNLLLDRNQGKCVEGGVEMFDMLLATSSRFRMMNLQGEEFVCLKSIILLNSGVYTFLSSTLKSLEEKDHIHRVLDKITDTLIHLMAKAGLTLQQQHQRLAQLLLILSHIRHMSNKGMEHLYSMKCKNVVPLYDLLLEMLDAHRLHAPTSRGGASVEETDQSHLATAGSTSSHSLQKYYITGEAEGFPATV

SEQ ID NO: 6 - protein, 314 aa, Artificial Sequence
  human sequence with point mutations
SAGDMRAANLWPSPLMIKRSKKNSLALSLTADQMVSALLDAEPPILYSEYDPTRPFSEASMMGLLTNLADRELVHMINWAKRVPGFVDLTLHDQVHLLECAWMEILMIGLVWRSMEHPGKLLWAPNLLLDRNQGKCVEGGVEMFDMLLATSSRFRMMNLQGEEFVCLKSIILLNSGVYTFLSSTLKSLEEKDHIHRVLDKITDTLIHLMAKAGLTLQQQHQRLAQLLLILSHIRHMSNKGMEHLYSMKCKNVVPLYDLLLEMLDAHRLHAPTSRGGASVEETDQSHLATAGSTSSHSLQKYYITGEAEGFPATV

SEQ ID NO: 7 - protein, 314 aa, Artificial Sequence
  human sequence with point mutations
SAGDMRAANLWPSPLMIKRSKKNSLALSLTADQMVSALLDAEPPILYSEYDPTRPFSEASMMGLLTNLADRELVHMINWAKRVPGFVDLTLHDQVHLLECAWMEILMIGMVWRSMEHPGKLLWAPNLLLDRNQGKCVEGGVEMFDMLLATSSRFRMMNLQGEEFVCLKSIILLNSGVYTFLSSTLKSLEEKDHIHRVLDKITDTLIHLMAKAGLTLQQQHQRLAQLLLILSHIRHMSNKGMEHLYSMKCKNVVPLYDLLLEMLDAHRLHAPTSRGGASVEETDQSHLATAGSTSSHSLQKYYITGEAEGFPATV

SEQ ID NO: 8 - protein, 314 aa, Artificial Sequence
  human sequence with point mutations
SAGDMRAANLWPSPLMIKRSKKNSLALSLTADQMVSALLDAEPPILYSEYDPTRPFSEASMMGLLTNLADRELVHMINWAKRVPGFVDLTLHDQVHLLECAWMEILMIGLVWRSMEHPGKLLFAPNLLLDRNQGKCVEGGVEVFDMLLATSSRFRMMNLQGEEFVCLKSIILLNSGVYTFLSSTLKSLEEKDHIHRVLDKITDTLIHLMAKAGLTLQQQHQRLAQLLLILSHIRHMSNKGMEHLYSMKCKNVVPLYDLLLEMLDAHRLHAPTSRGGASVEETDQSHLATAGSTSSHSLQKYYITGEAEGFPATV

SEQ ID NO: 9 - protein, 314 aa, Artificial Sequence
  human sequence with point mutations
SAGDMRAANLWPSPLMIKRSKKNSLALSLTADQMVSALLDAEPPILYSEYDPTRPFSEASMMGLLTNLADRELVHMINWAKRVPGFVDLTLHDQVHLLECAWMEILMIGLVWRSMEHPGKLLWAPNLLLDRNQGKCVEGGVEVFDMLLATSSRFRMMNLQGEEFVCLKSIILLNSGVYTFLSSTLKSLEEKDHIHRVLDKITDTLIHLMAKAGLTLQQQHQRLAQLLLILSHIRHMSNKGMEHLYSMKCKNVVPLYDLLLEMLDAHRLHAPTSRGGASVEETDQSHLATAGSTSSHSLQKYYITGEAEGFPATV

SEQ ID NO: 10 - protein, 314 aa, Artificial Sequence
  human sequence with point mutations
SAGDMRAANLWPSPLMIKRSKKNSLALSLTADQMVSALLDAEPPILYSEYDPTRPFSEASMMGLLTNLADRELVHMINWAKRVPGFVDLTLHDQVHLLECAWMEILMIGIVWRSMEHPGKLLFAPNLLLDRNQGKCVEGGVEVFDMLLATSSRFRMMNLQGEEFVCLKSIILLNSGVYTFLSSTLKSLEEKDHIHRVLDKITDTLIHLMAKAGLTLQQQHQRLAQLLLILSHIRHMSNKGMEHLYSMKCKNVVPLYDLLLEMLDAHRLHAPTSRGGASVEETDQSHLATAGSTSSHSLQKYYITGEAEGFPATV

SEQ ID NO: 11 - protein, 314 aa, Artificial Sequence
  human sequence with point mutations
SAGDMRAANLWPSPLMIKRSKKNSLALSLTADQMVSALLDAEPPILYSEYDPTRPFSEASMMGLLTNLADRELVHMINWAKRVPGFVDLTLHDQVHLLECAWMEILMIGLVWRSMEHPGKLLFAPNLLLDRNQGKCVEGGVELFDMLLATSSRFRMMNLQGEEFVCLKSIILLNSGVYTFLSSTLKSLEEKDHIHRVLDKITDTLIHLMAKAGLTLQQQHQRLAQLLLILSHIRHMSNKGMEHLYSMKCKNVVPLYDLLLEMLDAHRLHAPTSRGGASVEETDQSHLATAGSTSSHSLQKYYITGEAEGFPATV

SEQ ID NO: 12 - protein, 314 aa, Artificial Sequence
  human sequence with point mutations
SAGDMRAANLWPSPLMIKRSKKNSLALSLTADQMVSALLDAEPPILYSEYDPTRPFSEASMMGLLTNLADRELVHMINWAKRVPGFVDLTLHDQVHLLECAWMEILMIGLVWRSMEHPGKLLWAPNLLLDRNQGKCVEGGVELFDMLLATSSRFRMMNLQGEEFVCLKSIILLNSGVYTFLSSTLKSLEEKDHIHRVLDKITDTLIHLMAKAGLTLQQQHQRLAQLLLILSHIRHMSNKGMEHLYSMKCKNVVPLYDLLLEMLDAHRLHAPTSRGGASVEETDQSHLATAGSTSSHSLQKYYITGEAEGFPATV

SEQ ID NO: 13 - protein, 314 aa, Artificial Sequence
  human sequence with point mutations
SAGDMRAANLWPSPLMIKRSKKNSLALSLTADQMVSALLDAEPPILYSEYDPTRPFSEASMMGLLTNLADRELVHMINWAKRVPGFVDLTLHDQVHLLECAWMEILMIGVVWRSMEHPGKLLWAPNLLLDRNQGKCVEGGVELFDMLLATSSRFRMMNLQGEEFVCLKSIILLNSGVYTFLSSTLKSLEEKDHIHRVLDKITDTLIHLMAKAGLTLQQQHQRLAQLLLILSHIRHMSNKGMEHLYSMKCKNVVPLYDLLLEMLDAHRLHAPTSRGGASVEETDQSHLATAGSTSSHSLQKYYITGEAEGFPATV

SEQ ID NO: 14 - protein, 314 aa, Artificial Sequence
  human sequence with point mutations
SAGDMRAANLWPSPLMIKRSKKNSLALSLTADQMVSALLDAEPPILYSEYDPTRPFSEASMMGLLTNLADRELVHMINWAKRVPGFVDLTLHDQVHLLECAWMEILMIGLVWRSMEHPGKLLWAPNLLLDRNQGKCVEGAVELFDMLLATSSRFRMMNLQGEEFVCLKSIILLNSGVYTFLSSTLKSLEEKDHIHRVLDKITDTLIHLMAKAGLTLQQQHQRLAQLLLILSHIRHMSNKGMEHLYSMKCKNVVPLYDLLLEMLDAHRLHAPTSRGGASVEETDQSHLATAGSTSSHSLQKYYITGEAEGFPATV

SEQ ID NO: 15 - protein, 314 aa, Artificial Sequence
  human sequence with point mutations
SAGDMRAANLWPSPLMIKRSKKNSLALSLTADQMVSALLDAEPPILYSEYDPTRPFSEASMMGLLTNLADRELVHMINWAKRVPGFVDLTLHDQVHLLECAWMEILMIGVVWRSMEHPGKLLWAPNLLLDRNQGKCVEGAVELFDMLLATSSRFRMMNLQGEEFVCLKSIILLNSGVYTFLSSTLKSLEEKDHIHRVLDKITDTLIHLMAKAGLTLQQQHQRLAQLLLILSHIRHMSNKGMEHLYSMKCKNVVPLYDLLLEMLDAHRLHAPTSRGGASVEETDQSHLATAGSTSSHSLQKYYITGEAEGFPATV

SEQ ID NO: 16 - protein, 314 aa, Artificial Sequence
  human sequence with point mutations
SAGDMRAANLWPSPLMIKRSKKNSLALSLTADQMVSALLDAEPPILYSEYDPTRPFSEASMMGLLTNLAARELVHMINWAKRVPGFVDLTLHDQVHLLECAWMEILMIGLVWRSMEHPGKLLFAPNLLLDRNQGKCVEGGVEIFDMLLATSSRFRMMNLQGEEFVCLKSIILLNSGVYTFLSSTLKSLEEKDHIHRVLDKITDTLIHLMAKAGLTLQQQHQRLAQLLLILSHIRHMSNKGMEVLYSMKCKNVVPLYDLLLEMLDAHRLHAPTSRGGASVEETDQSHLATAGSTSSHSLQKYYITGEAEGFPATV

SEQ ID NO: 17 - protein, 314 aa, Artificial Sequence
  human sequence with point mutations
SAGDMRAANLWPSPLMIKRSKKNSLALSLTADQMVSALLDAEPPILYSEYDPTRPFSEASMMGLLTNLAARELVHMINWAKRVPGFVDLTLHDQVHLLECAWMEILMIGLVWRSMEHPGKLLWAPNLLLDRNQGKCVEGGVEIFDMLLATSSRFRMMNLQGEEFVCLKSIILLNSGVYTFLSSTLKSLEEKDHIHRVLDKITDTLIHLMAKAGLTLQQQHQRLAQLLLILSHIRHMSNKGMEVLYSMKCKNVVPLYDLLLEMLDAHRLHAPTSRGGASVEETDQSHLATAGSTSSHSLQKYYITGEAEGFPATV

SEQ ID NO: 18 - protein, 314 aa, Artificial Sequence
  human sequence with point mutations
SAGDMRAANLWPSPLMIKRSKKNSLALSLTADQMVSALLDAEPPILYSEYDPTRPFSEASMMGLLTNLAARELVHMINWAKRVPGFVDLTLHDQVHLLECAWMEILMIGLVWRSMEHPGKLLFAPNLLLDRNQGKCVEGGVEMFDMLLATSSRFRMMNLQGEEFVCLKSIILLNSGVYTFLSSTLKSLEEKDHIHRVLDKITDTLIHLMAKAGLTLQQQHQRLAQLLLILSHIRHMSNKGMEVLYSMKCKNVVPLYDLLLEMLDAHRLHAPTSRGGASVEETDQSHLATAGSTSSHSLQKYYITGEAEGFPATV

SEQ ID NO: 19 - protein, 314 aa, Artificial Sequence
  human sequence with point mutations
SAGDMRAANLWPSPLMIKRSKKNSLALSLTADQMVSALLDAEPPILYSEYDPTRPFSEASMMGLLTNLAARELVHMINWAKRVPGFVDLTLHDQVHLLECAWMEILMIGLVWRSMEHPGKLLWAPNLLLDRNQGKCVEGGVEMFDMLLATSSRFRMMNLQGEEFVCLKSIILLNSGVYTFLSSTLKSLEEKDHIHRVLDKITDTLIHLMAKAGLTLQQQHQRLAQLLLILSHIRHMSNKGMEVLYSMKCKNVVPLYDLLLEMLDAHRLHAPTSRGGASVEETDQSHLATAGSTSSHSLQKYYITGEAEGFPATV

SEQ ID NO: 20 - protein, 314 aa, Artificial Sequence
  human sequence with point mutations
SAGDMRAANLWPSPLMIKRSKKNSLALSLTADQMVSALLDAEPPILYSEYDPTRPFSEASMMGLLTNLAARELVHMINWAKRVPGFVDLTLHDQVHLLECAWMEILMIGMVWRSMEHPGKLLWAPNLLLDRNQGKCVEGGVEMFDMLLATSSRFRMMNLQGEEFVCLKSIILLNSGVYTFLSSTLKSLEEKDHIHRVLDKITDTLIHLMAKAGLTLQQQHQRLAQLLLILSHIRHMSNKGMEVLYSMKCKNVVPLYDLLLEMLDAHRLHAPTSRGGASVEETDQSHLATAGSTSSHSLQKYYITGEAEGFPATV

SEQ ID NO: 21 - protein, 314 aa, Artificial Sequence
  human sequence with point mutations
SAGDMRAANLWPSPLMIKRSKKNSLALSLTADQMVSALLDAEPPILYSEYDPTRPFSEASMMGLLTNLAARELVHMINWAKRVPGFVDLTLHDQVHLLECAWMEILMIGLVWRSMEHPGKLLFAPNLLLDRNQGKCVEGGVEVFDMLLATSSRFRMMNLQGEEFVCLKSIILLNSGVYTFLSSTLKSLEEKDHIHRVLDKITDTLIHLMAKAGLTLQQQHQRLAQLLLILSHIRHMSNKGMEVLYSMKCKNVVPLYDLLLEMLDAHRLHAPTSRGGASVEETDQSHLATAGSTSSHSLQKYYITGEAEGFPATV

SEQ ID NO: 22 - protein, 314 aa, Artificial Sequence
  human sequence with point mutations
SAGDMRAANLWPSPLMIKRSKKNSLALSLTADQMVSALLDAEPPILYSEYDPTRPFSEASMMGLLTNLAARELVHMINWAKRVPGFVDLTLHDQVHLLECAWMEILMIGLVWRSMEHPGKLLWAPNLLLDRNQGKCVEGGVEVFDMLLATSSRFRMMNLQGEEFVCLKSIILLNSGVYTFLSSTLKSLEEKDHIHRVLDKITDTLIHLMAKAGLTLQQQHQRLAQLLLILSHIRHMSNKGMEVLYSMKCKNVVPLYDLLLEMLDAHRLHAPTSRGGASVEETDQSHLATAGSTSSHSLQKYYITGEAEGFPATV

SEQ ID NO: 23 - protein, 314 aa, Artificial Sequence
  human sequence with point mutations
SAGDMRAANLWPSPLMIKRSKKNSLALSLTADQMVSALLDAEPPILYSEYDPTRPFSEASMMGLLTNLAARELVHMINWAKRVPGFVDLTLHDQVHLLECAWMEILMIGIVWRSMEHPGKLLFAPNLLLDRNQGKCVEGGVEVFDMLLATSSRFRMMNLQGEEFVCLKSIILLNSGVYTFLSSTLKSLEEKDHIHRVLDKITDTLIHLMAKAGLTLQQQHQRLAQLLLILSHIRHMSNKGMEVLYSMKCKNVVPLYDLLLEMLDAHRLHAPTSRGGASVEETDQSHLATAGSTSSHSLQKYYITGEAEGFPATV

SEQ ID NO: 24 - protein, 314 aa, Artificial Sequence
  human sequence with point mutations
SAGDMRAANLWPSPLMIKRSKKNSLALSLTADQMVSALLDAEPPILYSEYDPTRPFSEASMMGLLTNLAARELVHMINWAKRVPGFVDLTLHDQVHLLECAWMEILMIGLVWRSMEHPGKLLFAPNLLLDRNQGKCVEGGVELFDMLLATSSRFRMMNLQGEEFVCLKSIILLNSGVYTFLSSTLKSLEEKDHIHRVLDKITDTLIHLMAKAGLTLQQQHQRLAQLLLILSHIRHMSNKGMEVLYSMKCKNVVPLYDLLLEMLDAHRLHAPTSRGGASVEETDQSHLATAGSTSSHSLQKYYITGEAEGFPATV

SEQ ID NO: 25 - protein, 314 aa, Artificial Sequence
  human sequence with point mutations
SAGDMRAANLWPSPLMIKRSKKNSLALSLTADQMVSALLDAEPPILYSEYDPTRPFSEASMMGLLTNLAARELVHMINWAKRVPGFVDLTLHDQVHLLECAWMEILMIGLVWRSMEHPGKLLWAPNLLLDRNQGKCVEGGVELFDMLLATSSRFRMMNLQGEEFVCLKSIILLNSGVYTFLSSTLKSLEEKDHIHRVLDKITDTLIHLMAKAGLTLQQQHQRLAQLLLILSHIRHMSNKGMEVLYSMKCKNVVPLYDLLLEMLDAHRLHAPTSRGGASVEETDQSHLATAGSTSSHSLQKYYITGEAEGFPATV

SEQ ID NO: 26 - protein, 314 aa, Artificial Sequence
  human sequence with point mutations
SAGDMRAANLWPSPLMIKRSKKNSLALSLTADQMVSALLDAEPPILYSEYDPTRPFSEASMMGLLTNLAARELVHMINWAKRVPGFVDLTLHDQVHLLECAWMEILMIGVVWRSMEHPGKLLWAPNLLLDRNQGKCVEGGVELFDMLLATSSRFRMMNLQGEEFVCLKSIILLNSGVYTFLSSTLKSLEEKDHIHRVLDKITDTLIHLMAKAGLTLQQQHQRLAQLLLILSHIRHMSNKGMEVLYSMKCKNVVPLYDLLLEMLDAHRLHAPTSRGGASVEETDQSHLATAGSTSSHSLQKYYITGEAEGFPATV

SEQ ID NO: 27 - protein, 314 aa, Artificial Sequence
  human sequence with point mutations
SAGDMRAANLWPSPLMIKRSKKNSLALSLTADQMVSALLDAEPPILYSEYDPTRPFSEASMMGLLTNLAARELVHMINWAKRVPGFVDLTLHDQVHLLECAWMEILMIGLVWRSMEHPGKLLWAPNLLLDRNQGKCVEGAVELFDMLLATSSRFRMMNLQGEEFVCLKSIILLNSGVYTFLSSTLKSLEEKDHIHRVLDKITDTLIHLMAKAGLTLQQQHQRLAQLLLILSHIRHMSNKGMEVLYSMKCKNVVPLYDLLLEMLDAHRLHAPTSRGGASVEETDQSHLATAGSTSSHSLQKYYITGEAEGFPATV

SEQ ID NO: 28 - protein, 314 aa, Artificial Sequence
  human sequence with point mutations
SAGDMRAANLWPSPLMIKRSKKNSLALSLTADQMVSALLDAEPPILYSEYDPTRPFSEASMMGLLTNLAARELVHMINWAKRVPGFVDLTLHDQVHLLECAWMEILMIGVVWRSMEHPGKLLWAPNLLLDRNQGKCVEGAVELFDMLLATSSRFRMMNLQGEEFVCLKSIILLNSGVYTFLSSTLKSLEEKDHIHRVLDKITDTLIHLMAKAGLTLQQQHQRLAQLLLILSHIRHMSNKGMEVLYSMKCKNVVPLYDLLLEMLDAHRLHAPTSRGGASVEETDQSHLATAGSTSSHSLQKYYITGEAEGFPATV

SEQ ID NO: 29 - protein, 314 aa, Artificial Sequence
  human sequence with point mutations
SAGDMRAANLWPSPLMIKRSKKNSLALSLTADQMVSALLDAEPPILYSEYDPTRPFSEASMMGLLTNLADRELVHMINWAKRVPGFVDLTLHDQVHLLECAWMEILMIGLVWRSMEHPGKLLFAPNLLLDRNQGKCVEGGVEIFDMLLATSSRFRMMNLQGEEFVCLKSIILLNSGVYTFLSSTLKSLEEKDHIHRVLDKITDTLIHLMAKAGLTLQQQHQRLAQLLLILSHIRHMSNKRMEHLYSMKCKNVVPLYDLLLEMLDAHRLHAPTSRGGASVEETDQSHLATAGSTSSHSLQKYYITGEAEGFPATV

SEQ ID NO: 30 - protein, 314 aa, Artificial Sequence
  human sequence with point mutations
SAGDMRAANLWPSPLMIKRSKKNSLALSLTADQMVSALLDAEPPILYSEYDPTRPFSEASMMGLLTNLADRELVHMINWAKRVPGFVDLTLHDQVHLLECAWMEILMIGLVWRSMEHPGKLLWAPNLLLDRNQGKCVEGGVEIFDMLLATSSRFRMMNLQGEEFVCLKSIILLNSGVYTFLSSTLKSLEEKDHIHRVLDKITDTLIHLMAKAGLTLQQQHQRLAQLLLILSHIRHMSNKRMEHLYSMKCKNVVPLYDLLLEMLDAHRLHAPTSRGGASVEETDQSHLATAGSTSSHSLQKYYITGEAEGFPATV

SEQ ID NO: 31 - protein, 314 aa, Artificial Sequence
  human sequence with point mutations
SAGDMRAANLWPSPLMIKRSKKNSLALSLTADQMVSALLDAEPPILYSEYDPTRPFSEASMMGLLTNLADRELVHMINWAKRVPGFVDLTLHDQVHLLECAWMEILMIGLVWRSMEHPGKLLFAPNLLLDRNQGKCVEGGVEMFDMLLATSSRFRMMNLQGEEFVCLKSIILLNSGVYTFLSSTLKSLEEKDHIHRVLDKITDTLIHLMAKAGLTLQQQHQRLAQLLLILSHIRHMSNKRMEHLYSMKCKNVVPLYDLLLEMLDAHRLHAPTSRGGASVEETDQSHLATAGSTSSHSLQKYYITGEAEGFPATV

SEQ ID NO: 32 - protein, 314 aa, Artificial Sequence
  human sequence with point mutations
SAGDMRAANLWPSPLMIKRSKKNSLALSLTADQMVSALLDAEPPILYSEYDPTRPFSEASMMGLLTNLADRELVHMINWAKRVPGFVDLTLHDQVHLLECAWMEILMIGLVWRSMEHPGKLLWAPNLLLDRNQGKCVEGGVEMFDMLLATSSRFRMMNLQGEEFVCLKSIILLNSGVYTFLSSTLKSLEEKDHIHRVLDKITDTLIHLMAKAGLTLQQQHQRLAQLLLILSHIRHMSNKRMEHLYSMKCKNVVPLYDLLLEMLDAHRLHAPTSRGGASVEETDQSHLATAGSTSSHSLQKYYITGEAEGFPATV

SEQ ID NO: 33 - protein, 314 aa, Artificial Sequence
  human sequence with point mutations
SAGDMRAANLWPSPLMIKRSKKNSLALSLTADQMVSALLDAEPPILYSEYDPTRPFSEASMMGLLTNLADRELVHMINWAKRVPGFVDLTLHDQVHLLECAWMEILMIGMVWRSMEHPGKLLWAPNLLLDRNQGKCVEGGVEMFDMLLATSSRFRMMNLQGEEFVCLKSIILLNSGVYTFLSSTLKSLEEKDHIHRVLDKITDTLIHLMAKAGLTLQQQHQRLAQLLLILSHIRHMSNKRMEHLYSMKCKNVVPLYDLLLEMLDAHRLHAPTSRGGASVEETDQSHLATAGSTSSHSLQKYYITGEAEGFPATV

SEQ ID NO: 34 - protein, 314 aa, Artificial Sequence
  human sequence with point mutations
SAGDMRAANLWPSPLMIKRSKKNSLALSLTADQMVSALLDAEPPILYSEYDPTRPFSEASMMGLLTNLADRELVHMINWAKRVPGFVDLTLHDQVHLLECAWMEILMIGLVWRSMEHPGKLLFAPNLLLDRNQGKCVEGGVEVFDMLLATSSRFRMMNLQGEEFVCLKSIILLNSGVYTFLSSTLKSLEEKDHIHRVLDKITDTLIHLMAKAGLTLQQQHQRLAQLLLILSHIRHMSNKRMEHLYSMKCKNVVPLYDLLLEMLDAHRLHAPTSRGGASVEETDQSHLATAGSTSSHSLQKYYITGEAEGFPATV

SEQ ID NO: 35 - protein, 314 aa, Artificial Sequence
  human sequence with point mutations
SAGDMRAANLWPSPLMIKRSKKNSLALSLTADQMVSALLDAEPPILYSEYDPTRPFSEASMMGLLTNLADRELVHMINWAKRVPGFVDLTLHDQVHLLECAWMEILMIGLVWRSMEHPGKLLWAPNLLLDRNQGKCVEGGVEVFDMLLATSSRFRMMNLQGEEFVCLKSIILLNSGVYTFLSSTLKSLEEKDHIHRVLDKITDTLIHLMAKAGLTLQQQHQRLAQLLLILSHIRHMSNKRMEHLYSMKCKNVVPLYDLLLEMLDAHRLHAPTSRGGASVEETDQSHLATAGSTSSHSLQKYYITGEAEGFPATV

SEQ ID NO: 36 - protein, 314 aa, Artificial Sequence
  human sequence with point mutations
SAGDMRAANLWPSPLMIKRSKKNSLALSLTADQMVSALLDAEPPILYSEYDPTRPFSEASMMGLLTNLADRELVHMINWAKRVPGFVDLTLHDQVHLLECAWMEILMIGIVWRSMEHPGKLLFAPNLLLDRNQGKCVEGGVEVFDMLLATSSRFRMMNLQGEEFVCLKSIILLNSGVYTFLSSTLKSLEEKDHIHRVLDKITDTLIHLMAKAGLTLQQQHQRLAQLLLILSHIRHMSNKRMEHLYSMKCKNVVPLYDLLLEMLDAHRLHAPTSRGGASVEETDQSHLATAGSTSSHSLQKYYITGEAEGFPATV

SEQ ID NO: 37 - protein, 314 aa, Artificial Sequence
  human sequence with point mutations
SAGDMRAANLWPSPLMIKRSKKNSLALSLTADQMVSALLDAEPPILYSEYDPTRPFSEASMMGLLTNLADRELVHMINWAKRVPGFVDLTLHDQVHLLECAWMEILMIGLVWRSMEHPGKLLFAPNLLLDRNQGKCVEGGVELFDMLLATSSRFRMMNLQGEEFVCLKSIILLNSGVYTFLSSTLKSLEEKDHIHRVLDKITDTLIHLMAKAGLTLQQQHQRLAQLLLILSHIRHMSNKRMEHLYSMKCKNVVPLYDLLLEMLDAHRLHAPTSRGGASVEETDQSHLATAGSTSSHSLQKYYITGEAEGFPATV

SEQ ID NO: 38 - protein, 314 aa, Artificial Sequence
  human sequence with point mutations
SAGDMRAANLWPSPLMIKRSKKNSLALSLTADQMVSALLDAEPPILYSEYDPTRPFSEASMMGLLTNLADRELVHMINWAKRVPGFVDLTLHDQVHLLECAWMEILMIGLVWRSMEHPGKLLWAPNLLLDRNQGKCVEGGVELFDMLLATSSRFRMMNLQGEEFVCLKSIILLNSGVYTFLSSTLKSLEEKDHIHRVLDKITDTLIHLMAKAGLTLQQQHQRLAQLLLILSHIRHMSNKRMEHLYSMKCKNVVPLYDLLLEMLDAHRLHAPTSRGGASVEETDQSHLATAGSTSSHSLQKYYITGEAEGFPATV

SEQ ID NO: 39 - protein, 314 aa, Artificial Sequence
  human sequence with point mutations
SAGDMRAANLWPSPLMIKRSKKNSLALSLTADQMVSALLDAEPPILYSEYDPTRPFSEASMMGLLTNLADRELVHMINWAKRVPGFVDLTLHDQVHLLECAWMEILMIGVVWRSMEHPGKLLWAPNLLLDRNQGKCVEGGVELFDMLLATSSRFRMMNLQGEEFVCLKSIILLNSGVYTFLSSTLKSLEEKDHIHRVLDKITDTLIHLMAKAGLTLQQQHQRLAQLLLILSHIRHMSNKRMEHLYSMKCKNVVPLYDLLLEMLDAHRLHAPTSRGGASVEETDQSHLATAGSTSSHSLQKYYITGEAEGFPATV

SEQ ID NO: 40 - protein, 314 aa, Artificial Sequence
  human sequence with point mutations
SAGDMRAANLWPSPLMIKRSKKNSLALSLTADQMVSALLDAEPPILYSEYDPTRPFSEASMMGLLTNLADRELVHMINWAKRVPGFVDLTLHDQVHLLECAWMEILMIGLVWRSMEHPGKLLWAPNLLLDRNQGKCVEGAVELFDMLLATSSRFRMMNLQGEEFVCLKSIILLNSGVYTFLSSTLKSLEEKDHIHRVLDKITDTLIHLMAKAGLTLQQQHQRLAQLLLILSHIRHMSNKRMEHLYSMKCKNVVPLYDLLLEMLDAHRLHAPTSRGGASVEETDQSHLATAGSTSSHSLQKYYITGEAEGFPATV

SEQ ID NO: 41 - protein, 314 aa, Artificial Sequence
  human sequence with point mutations
SAGDMRAANLWPSPLMIKRSKKNSLALSLTADQMVSALLDAEPPILYSEYDPTRPFSEASMMGLLTNLADRELVHMINWAKRVPGFVDLTLHDQVHLLECAWMEILMIGVVWRSMEHPGKLLWAPNLLLDRNQGKCVEGAVELFDMLLATSSRFRMMNLQGEEFVCLKSIILLNSGVYTFLSSTLKSLEEKDHIHRVLDKITDTLIHLMAKAGLTLQQQHQRLAQLLLILSHIRHMSNKRMEHLYSMKCKNVVPLYDLLLEMLDAHRLHAPTSRGGASVEETDQSHLATAGSTSSHSLQKYYITGEAEGFPATV

SEQ ID NO: 42 - DNA, 14 bp, Artificial Sequence
  consensus human sequence
ggttaatattaata

SEQ ID NO: 43 - protein, 844 aa, Artificial Sequence [sequence omitted]

SEQ ID NO: 44 - protein, 7 aa, Human
MPKRPRP

SEQ ID NO: 45 - DNA, 32 bp, Artificial Sequence
  human sequence with added restriction site sequence
ggaattcgttgaccgggtctgctggagacatg

SEQ ID NO: 46 - DNA, 43 bp, Artificial Sequence
  human sequence with added restriction site sequence
ggaattcgagctctgaaccagacccgactgtggcagggaaacc

SEQ ID NO: 47 - DNA, 49 bp, Artificial Sequence
  human sequence with added restriction site sequence
gtccctgacggccgaccagatggtcagtgccttgttggatgctgagccc

SEQ ID NO: 48 - DNA, 51 bp, Artificial Sequence
  human sequence with added restriction site sequence
gtgctccatggagcgccagacgagaccaatcatcaggatctccatccaggc

SEQ ID NO: 49 - DNA, 32 bp, Artificial Sequence
  human sequence with added restriction site sequence
caaggcaggcctgaccctgcagcagcagcacc

SEQ ID NO: 50 - DNA, 74 bp, Artificial Sequence
  human sequence with added restriction site sequence
gcatctccagcagcaggtcatagaggggcaccacgttcttgcacttcatgctgtacagatgctccatcactttg

SEQ ID NO: 51 - DNA, 73 bp, Artificial Sequence
  human sequence with added restriction site sequence
gcatctccagcagcaggtcatagaggggcaccacgttcttgcacttcatgctgtacagcacctccatgccttt

SEQ ID NO: 52 - DNA, 50 bp, Artificial Sequence
  human sequence with added restriction site sequence
cttcagtgaagcttcgatgatgggcttactgaccaacctggcagccaggg

SEQ ID NO: 53 - DNA, 29 bp, Artificial Sequence
  human sequence with added restriction site sequence
ctatgacctgctgctggagatgctggacg

SEQ ID NO: 54 - DNA, 28 bp, Artificial Sequence
  human sequence with added restriction site sequence
catatggtcgacgaattcgcggccgcac

SEQ ID NO: 55 - DNA, 74 bp, Artificial Sequence
  human sequence with added restriction site sequence
gcatctccagcagcaggtcatagaggggcaccacgttcttgcacttcatgctgtacagatgctccatgcgtttg

SEQ ID NO: 56 - DNA, 29 bp, Artificial Sequence
  human sequence with added restriction site sequence
gtccaagatctccacgatgccctctacac

SEQ ID NO: 57 - DNA, 34 bp, Artificial Sequence
  human sequence with added restriction site sequence
gatatccaagaacagcctggccttgtccctgacg

SEQ ID NO: 58 - DNA, 41 bp, Artificial Sequence
  human sequence with added restriction site sequence
actagtgaattcgactgtggcagggaaaccctctgcctccc

SEQ ID NO: 59 - DNA, 20 bp, Artificial Sequence
  human sequence with added restriction site sequence
ctgaccaacctggcagacag

SEQ ID NO: 60 - DNA, 20 bp, Artificial Sequence
  human sequence with added restriction site sequence
ggactcggtggatatggtcc

SEQ ID NO: 61 - DNA, 21 bp, Artificial Sequence
  human sequence with added restriction site sequence
gttcacatgatcaactgggcg

SEQ ID NO: 62 - DNA, 20 bp, Artificial Sequence
  human sequence with added restriction site sequence
gagacttcagggtgctggac